(19) United States
(12) Patent Application Publication
(10) Pub. No.: US 2011/0178062 A1
(43) Pub. Date: Jul. 21, 2011

(54) INDOLE AND BENZOTHIOPHENE COMPOUNDS AS MODULATORS OF THE HISTAMINE H3 RECEPTOR

(76) Inventors: Brett D. Allison, Del Mar, CA (US); Cheryl A. Grice, Carlsbad, CA (US); Kelly J. McClure, Ramona, CA (US); Alejandro Santillan, JR., San Diego, CA (US)

(21) Appl. No.: 12/039,143

(22) Filed: Feb. 28, 2008

Related U.S. Application Data
(60) Provisional application No. 60/892,330, filed on Mar. 1, 2007.

(51) Int. Cl. A61K 31/553; C07D 401/14; A61K 31/551; A61K 31/4545; A61K 31/496; C07D 403/14; A61K 31/454; A61K 31/55; C07D 413/14; C07D 417/14; A61K 31/541; A61P 25/00

(52) U.S. Cl. 514/211.15; 540/575; 514/218; 546/187; 514/316; 544/364; 514/253.11; 546/201; 514/323; 540/598; 514/217.05; 544/58.4; 514/228.2; 540/544

(57) ABSTRACT

Certain substituted indole and benzothiophene compounds are histamine H₃ receptor modulators useful in the treatment of histamine H₃ receptor-mediated diseases.

### CROSS-REFERENCE TO RELATED APPLICATIONS

This application claims the benefit of U.S. Provisional Application 60/892,330, filed Mar. 1, 2007, which is hereby incorporated by reference in its entirety.

### FIELD OF THE INVENTION

The present invention relates to certain indole and benzothiophene compounds, pharmaceutical compositions containing them, and methods of using them for the treatment of disease states, disorders, and conditions mediated by the histamine H₃ receptor.

### BACKGROUND OF THE INVENTION

The histamine H₃ receptor was first described as a presynaptic autoreceptor in the central nervous system (CNS) (Arrang, J.-M. et al., Nature 1983, 302, 832-837) controlling the synthesis and release of histamine. The histamine H₃ receptor is primarily expressed in the mammalian central nervous system (CNS), with some minimal expression in peripheral tissues such as vascular smooth muscle.

Thus, several indications for histamine H₃ antagonists and inverse agonists have been proposed based on animal pharmacology and other experiments with known histamine H₃ antagonists (e.g. thioperamide). (See: Krause et al. and Phillips et al. in “The Histamine H₃ Receptor-A Target for New Drugs”, Leurs, R. and Timmerman, H., (Eds.), Elsevier, 1998, pp. 175-196 and 197-222; Morisset, S. et al., Nature 2000, 408, 860-864.) These include conditions such as cognitive disorders, sleep disorders, psychiatric disorders, and other disorders.

For example, histamine H₃ antagonists have been shown to have pharmacological activity relevant to several key symptoms of depression, including sleep disorders (e.g. sleep disturbances, fatigue, and lethargy) and cognitive difficulties (e.g. memory and concentration impairment), as described above. For reviews, see: Celanire, S. Drug Discovery Today 2005, 10(23/24), 1613-1627; Hancock, A. A. Biochem. Pharmacol. 2006, 71, 1103-1113; Bonaventure, P. et al. Biochem. Pharm. 2007, 73, 1084-1096; and Letavic, M. A. et al. Prog. Med. Chem. 1996, 44, 181-206. There remains a need for potent histamine H₃ receptor modulators with desirable pharmaceutical properties.

Certain indole compounds are described in: Zhang, H.-C. et al. Tetrahedron Lett. 1998, 39, 4449-4452; Intl. Pat. Appl. Publ. WO2003/106418; Intl. Pat. Appl. Publ. WO99/61426; Intl. Pat. Appl. Publ. WO 2000/012074; U.S. Pat. No. 6,723,725; and Pat. Appl. Publ. DE 2940687 (equivalent of U.S. Pat. No. 4,252,803). Indole benzoic acid derivatives are described as PPAR receptor antagonists in Intl. Pat. Appl. Publ. WO 01/12187. Indoles are described as histamine H₃ inverse agonists in U.S. Pat. Appl. Publ. US2006/0160855 and U.S. Pat. Appl. Publ. US2005/0282864. Indoles are described as histamine H₃ antagonists in Intl. Pat. Appl. Publ. Nos. WO2004/026837 and WO2008/015125.

### SUMMARY OF THE INVENTION

Certain indole and benzothiophene derivatives have now been found to have histamine H₃ receptor modulating activity. Thus, the invention is directed to the general and preferred embodiments defined, respectively, by the independent and dependent claims appended hereto, which are incorporated by reference herein.

In one general aspect the invention relates to a compound of the following Formula (I):

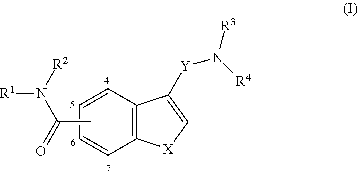

wherein
X is NR^{a} and Y is —CH₂— or X is S and Y is —CH₂— or —C(O)—;
   where R^{a} is —H, methyl, —SO₂methyl;
      the substituent —C(O)NR¹R² is bound at the 4-, 5-, 6-, or 7-position on Formula (I);
R¹ is —H and R² is —(CH₂)-pyridyl, where said pyridyl is unsubstituted or substituted with methyl;
or R¹ and R² taken together with the nitrogen to which they are attached form one of the following moieties:

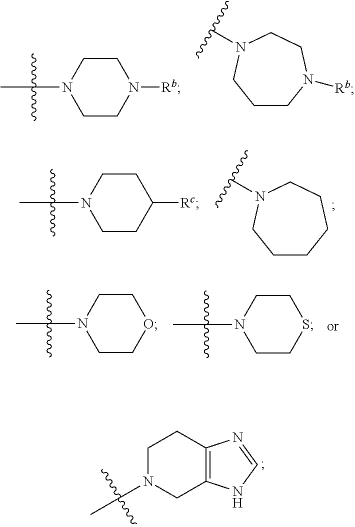

where R^{b} is isopropyl, cyclopropyl, or cyclobutyl; and
R^{c} is —H, hydroxymethyl, phenyl, or 1-pyrrolidin-2-onyl;
R³ and R⁴ taken together with the nitrogen to which they are attached form one of the following moieties:

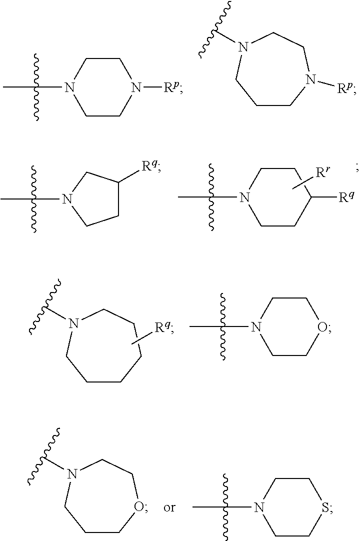

where R^{P} is isopropyl, acetyl, methylsulfonyl, C₃₋₅cycloalkyl, phenyl, —C(O)— phenyl, biphenyl, benzyl, benzhydryl, phenethyl, pyridyl, —C(O)-pyridyl, thiazolyl, or —C(O)-morpholinyl;
R^{q} is —H, —OH, phenyl, benzyl, —NR^{s}R^{t}, or —N(R^{s})C(O)R^{t};
   where R^{s} and R^{t} are each independently —H or methyl;
   or alternatively, R^{s} and R^{t} taken together with the nitrogen to which they are attached form piperidine; and
R^{r} is —H or —OH;
   with the following provisos:
   1) when
a) the substituent —C(O)NR¹R² is bound at the 5-position in Formula (I); and
b) R¹ and R² taken together with the nitrogen to which they are attached form one of the following moieties:

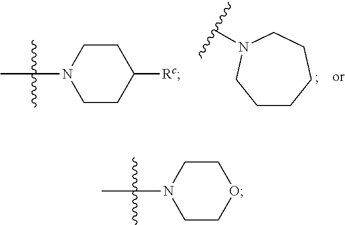

and
c) R^{c} is —H;
then R³ and R⁴ taken together with the nitrogen to which they are attached do not form one of the following moieties:

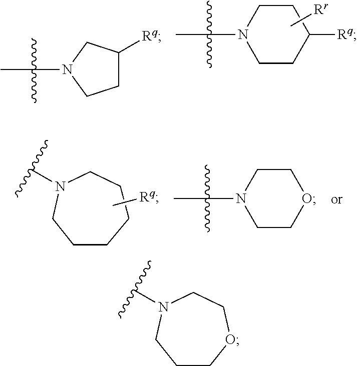

where R^{q} is —H and R^{r} is —H;
   2) when
a) X is NR^{a}; and
b) the substituent —C(O)NR¹R² is bound at the 4- or 7-position on Formula (I);
then the substituents —C(O)NR¹R² and —YNR³R⁴ together comprise two
nitrogens each of which is not adjacent to a carbonyl or sulfonyl group;
   3) when
a) NR¹R² is 4-benzylpiperidin-1-yl; and
b) the substituent —C(O)NR¹R² is bound at the 5- or 6-position on Formula (I);
then R³ and R⁴ taken together with the nitrogen to which they are attached do not form one of the following moieties:

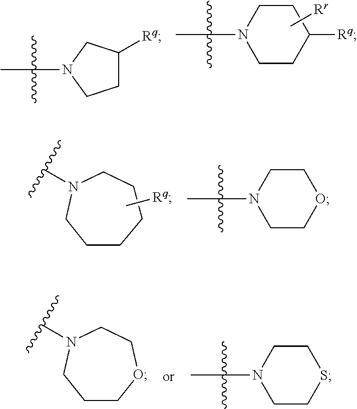

where R^{q} is —H and R^{r} is —H;
   or a pharmaceutically acceptable salt, a pharmaceutically acceptable prodrug, or a pharmaceutically active metabolite thereof.

In a further general aspect, the invention relates to pharmaceutical compositions each comprising: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

In another general aspect, the invention is directed to a method of treating a subject suffering from or diagnosed with a disease, disorder, or medical condition mediated by histamine H₃ receptor activity, comprising administering to the subject in need of such treatment an effective amount of a compound of Formula (I), or a pharmaceutically acceptable salt, pharmaceutically acceptable prodrug, or pharmaceutically active metabolite thereof.

In certain preferred embodiments of the inventive method, the disease, disorder, or medical condition is selected from: cognitive disorders, sleep disorders, psychiatric disorders, and other disorders.

Additional embodiments, features, and advantages of the invention will be apparent from the following detailed description and through practice of the invention.

### DETAILED DESCRIPTION

The invention may be more fully appreciated by reference to the following description, including the following glossary of terms and the concluding examples. For the sake of brevity, the disclosures of the publications, including patents, cited in this specification are herein incorporated by reference.

As used herein, the terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by /), ethyl (Et), n-propyl, isopropyl, butyl, isobutyl, sec-butyl, tert-butyl (tBu), pentyl, isopentyl, tert-pentyl, hexyl, isohexyl, and groups that in light of the ordinary skill in the art and the teachings provided herein would be considered equivalent to any one of the foregoing examples.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities, in the form of properly bonded moieties:

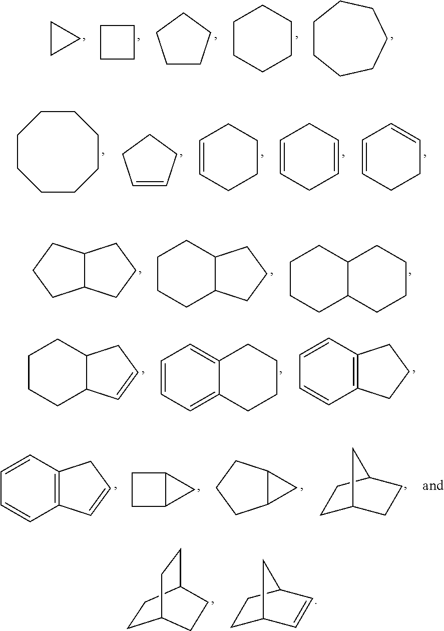

A “heterocycloalkyl” refers to a monocyclic ring structure that is saturated or partially saturated and has from 4 to 7 ring atoms per ring structure selected from carbon atoms and up to two heteroatoms selected from nitrogen, oxygen, and sulfur. The ring structure may optionally contain up to two oxo groups on sulfur ring members. Illustrative entities, in the form of properly bonded moieties, include:

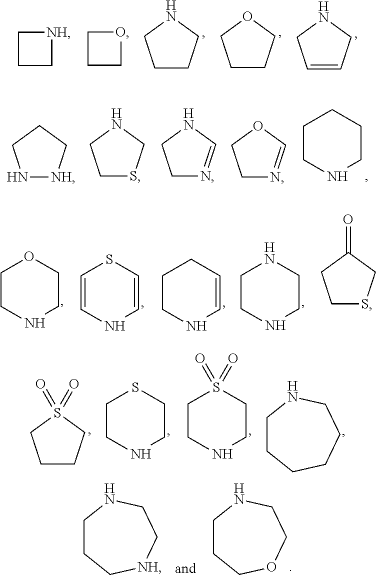

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities, in the form of properly bonded moieties:

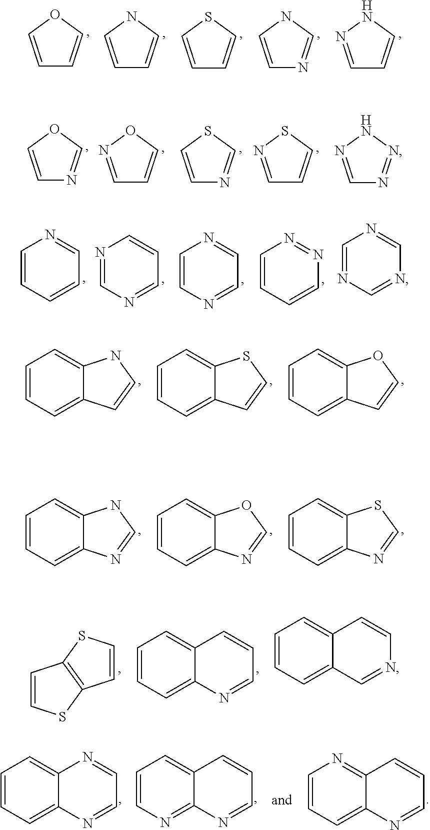

Those skilled in the art will recognize that the species of cycloalkyl, heterocycloalkyl, and heteroaryl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

Any formula given herein is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³²P, ³³P, ³⁵S, ¹⁸F, ³⁶Cl, and ¹²⁵I, respectively. Such isotopically labeled compounds are useful in metabolic studies (preferably with ¹⁴C), reaction kinetic studies (with, for example ²H or ³H), detection or imaging techniques [such as positron emission tomography (PET) or single-photon emission computed tomography (SPECT)] including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to any formula given herein, the selection of a particular moiety from a list of possible species for a specified variable is not intended to define the moiety for the variable appearing elsewhere. In other words, where a variable appears more than once, the choice of the species from a specified list is independent of the choice of the species for the same variable elsewhere in the formula.

In preferred embodiments of Formula (I), X is NR^{a} and Y is —CH₂—. In further preferred embodiments, X is S and Y is —C(O)—.

In preferred embodiments, R^{a} is —H.

In preferred embodiments, the substituent —C(O)NR¹R² is bound at the 5- or 6-position on Formula (I). In further preferred embodiments, the substituent —C(O)NR¹R² is bound at the 6-position on Formula (I).

In preferred embodiments, R¹ is —H and R² is pyridin-3-ylmethyl, pyridin-4-ylmethyl, or 3-methyl-pyridin-2-ylmethyl.

In preferred embodiments, R¹ and R² taken together with the nitrogen to which they are attached form one of the following moieties:

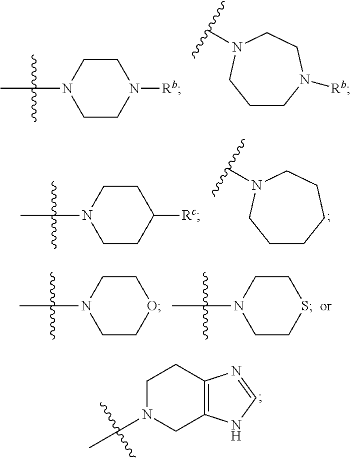

where R^{b} is cyclopropyl or cyclobutyl; and
R^{c} is hydroxymethyl, phenyl, or 1-pyrrolidin-2-onyl. In further preferred embodiments, R¹ and R² taken together with the nitrogen to which they are attached form

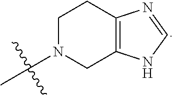

In preferred embodiments, R³ and R⁴ taken together with the nitrogen to which they are attached form one of the following moieties:

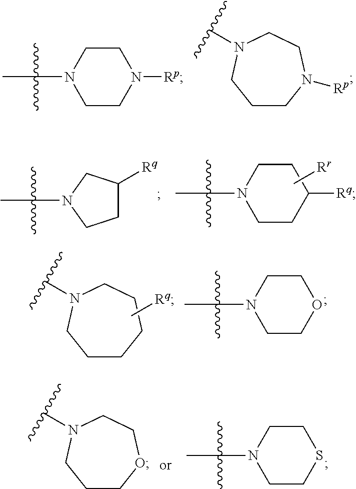

where R^{p}, R^{q}, and R^{r} are as defined in Formula (I). In further preferred embodiments, R³ and R⁴ taken together with the nitrogen to which they are attached form one of the following moieties:

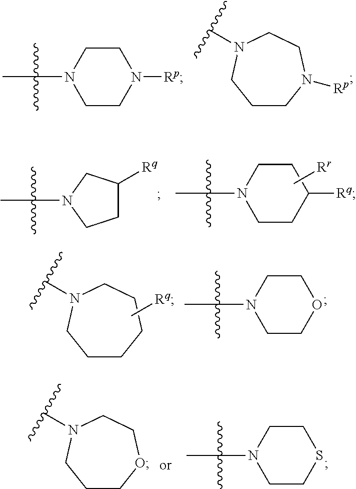

where R^{q} is —OH, phenyl, benzyl, —NR^{s}R^{t}, or —N(R^{s})C(O)R^{t};
and R^{P}, R^{r}, R^{s} and R^{t} are defined as in Formula (I).

In preferred embodiments, R^{p} is isopropyl, cyclopropyl, or cyclobutyl.

In preferred embodiments, R^{q} is —H.

In certain preferred embodiments, the compound of Formula (I) is selected from the group consisting of:

| Ex. | Compound Name |
|---|---|
| 1 | (4-Isopropyl-piperazin-1-yl)-(1-methyl-3-morpholin-4-ylmethyl-1H-indol- |
| | 6-yl)-methanone; |
| 2 | (4-Cyclopropyl-piperazin-1-yl)-(1-methyl-3-morpholin-4-ylmethyl-1H- |
| | indol-6-yl)-methanone; |
| 3 | (4-Cyclobutyl-piperazin-1-yl)-(1-methyl-3-morpholin-4-ylmethyl-1H- |
| | indol-6-yl)-methanone; |
| 4 | (4-Isopropyl-piperazin-1-yl)-(3-morpholin-4-ylmethyl-1H-indol-6-yl)- |
| | methanone; |
| 5 | (4-Cyclopropyl-piperazin-1-yl)-(3-morpholin-4-ylmethyl-1H-indol-6-yl)- |
| | methanone; |
| 6 | (4-Cyclobutyl-piperazin-1-yl)-(3-morpholin-4-ylmethyl-1H-indol-6-yl)- |
| | methanone; |
| 7 | (4-Isopropyl-[1,4]diazepan-1-yl)-(3-morpholin-4-ylmethyl-1H-indol-6-yl)- |
| | methanone; |
| 8 | (4-Cyclopropyl-[1,4]diazepan-1-yl)-(3-morpholin-4-ylmethyl-1H-indol-6- |
| | yl)-methanone; |
| 9 | (4-Cyclobutyl-[1,4]diazepan-1-yl)-(3-morpholin-4-ylmethyl-1H-indol-6- |
| | yl)-methanone; |
| 10 | 3-Morpholin-4-ylmethyl-1H-indole-6-carboxylic acid (pyridin-3-ylmethyl)-amide; |
| 11 | 3-Morpholin-4-ylmethyl-1H-indole-6-carboxylic acid (pyridin-4-ylmethyl)-amide; |
| 12 | 3-Morpholin-4-ylmethyl-1H-indole-6-carboxylic acid (3-methyl-pyridin-2- |
| | ylmethyl)-amide; |
| 13 | (3-Morpholin-4-ylmethyl-1H-indol-6-yl)-(3,4,6,7-tetrahydro-imidazo[4,5- |
| | c]pyridin-5-yl)-methanone; |
| 14 | 1-[1-(3-Morpholin-4-ylmethyl-1H-indole-6-carbonyl)-piperidin-4-yl]- |
| | pyrrolidin-2-one; |
| 15 | 3-Piperidin-1-ylmethyl-1H-indole-6-carboxylic acid (pyridin-3-ylmethyl)- |
| | amide; |
| 16 | 3-Piperidin-1-ylmethyl-1H-indole-6-carboxylic acid (pyridin-4-ylmethyl)- |
| | amide; |
| 17 | 3-Piperidin-1-ylmethyl-1H-indole-6-carboxylic acid (3-methyl-pyridin-2- |
| | ylmethyl)-amide; |
| 18 | (3-Piperidin-1-ylmethyl-1H-indol-6-yl)-(3,4,6,7-tetrahydro-imidazo[4,5- |
| | c]pyridin-5-yl)-methanone; |
| 19 | 1-[1-(3-Piperidin-1-ylmethyl-1H-indole-6-carbonyl)-piperidin-4-yl]- |
| | pyrrolidin-2-one; |
| 20 | (4-Isopropyl-piperazin-1-yl)-(3-morpholin-4-ylmethyl-1H-indol-7-yl)- |
| | methanone; |
| 21 | (4-Cyclobutyl-piperazin-1-yl)-(3-morpholin-4-ylmethyl-1H-indol-7-yl)- |
| | methanone; |
| 22 | (4-Isopropyl-piperazin-1-yl)-(3-piperidin-1-ylmethyl-1H-indol-7-yl)- |
| | methanone; |
| 23 | (4-Cyclobutyl-piperazin-1-yl)-(3-piperidin-1-ylmethyl-1H-indol-7-yl)- |
| | methanone; |
| 24 | (4-Isopropyl-piperazin-1-yl)-(3-piperidin-1-ylmethyl-1H-indol-6-yl)- |
| | methanone; |
| 25 | (4-Isopropyl-piperazin-1-yl)-(1-methanesulfonyl-3-piperidin-1-ylmethyl- |
| | 1H-indol-6-yl)-methanone; |
| 26 | (4-Isopropyl-piperazin-1-yl)-(1-methanesulfonyl-3-morpholin-4- |
| | ylmethyl-1H-indol-6-yl)-methanone; |
| 27 | [3-(4-Isopropyl-piperazin-1-ylmethyl)-1H-indol-6-yl]-piperidin-1-yl- |
| | methanone; |
| 28 | [3-(4-Cyclopropyl-piperazin-1-ylmethyl)-1H-indol-6-yl]-piperidin-1-yl- |
| | methanone; |
| 29 | [3-(4-Cyclobutyl-piperazin-1-ylmethyl)-1H-indol-6-yl]-piperidin-1-yl- |
| | methanone; |
| 30 | [3-(4-Isopropyl-[1,4]diazepan-1-ylmethyl)-1H-indol-6-yl]-piperidin-1-yl- |
| | methanone; |
| 31 | [3-(4-Cyclopropyl-[1,4]diazepan-1-ylmethyl)-1H-indol-6-yl]-piperidin-1- |
| | yl-methanone; |
| 32 | (4-Isopropyl-piperazin-1-yl)-(3-piperidin-1-ylmethyl-1H-indol-5-yl)- |
| | methanone; |
| 33 | (4-Cyclobutyl-piperazin-1-yl)-(3-piperidin-1-ylmethyl-1H-indol-5-yl)- |
| | methanone; |
| 34 | (4-Isopropyl-[1,4]diazepan-1-yl)-(3-piperidin-1-ylmethyl-1H-indol-5-yl)- |
| | methanone; |
| 35 | (4-Isopropyl-piperazin-1-yl)-(3-morpholin-4-ylmethyl-1H-indol-5-yl)- |
| | methanone; |
| 36 | [3-(4-Cyclobutyl-piperazin-1-ylmethyl)-1H-indol-7-yl]-piperidin-1-yl- |
| | methanone; |
| 37 | [3-(4-Cyclobutyl-piperazin-1-ylmethyl)-1H-indol-4-yl]-piperidin-1-yl- |
| | methanone; |
| 38 | [3-(4-Cyclobutyl-piperazin-1-ylmethyl)-1H-indol-5-yl]-piperidin-1-yl- |
| | methanone; |
| 39 | (4-Isopropyl-piperazin-1-yl)-(3-morpholin-4-ylmethyl-1H-indol-4-yl)- |
| | methanone; |
| 40 | (4-Cyclobutyl-piperazin-1-yl)-(3-morpholin-4-ylmethyl-1H-indol-5-yl)- |
| | methanone; |
| 41 | (4-Cyclobutyl-piperazin-1-yl)-(3-morpholin-4-ylmethyl-1H-indol-4-yl)- |
| | methanone; |
| 42 | (4-Cyclobutyl-piperazin-1-yl)-(3-piperidin-1-ylmethyl-1H-indol-4-yl)- |
| | methanone; |
| 43 | [3-(4-Cyclobutyl-piperazine-1-ylmethyl)-1H-indol-6-yl]-(4-phenyl- |
| | piperidin-1-yl)-methanone; |
| 44 | [3-(3-Hydroxymethyl-piperidin-1-ylmethyl)-1H-indol-6-yl]-(4-isopropyl- |
| | piperazin-1-yl)-methanone; |
| 45 | (4-Isopropyl-piperazin-1-yl)-[3-(4-phenyl-piperazin-1-ylmethyl)-1H- |
| | indol-6-yl]-methanone; |
| 46 | (4-Isopropyl-piperazin-1-yl)-[3-(4-pyridin-2-yl-piperazin-1-ylmethyl)-1H- |
| | indol-6-yl]-methanone; |
| 47 | (4-Isopyropyl-piperazin-1-yl)-(3-thiomorpholin-4-ylmethyl-1H-indol-6-yl)- |
| | methanone; |
| 48 | 1-{4-[6-(4-Isopropyl-piperazine-1-carbonyl)-1H-indol-3-ylmethyl]- |
| | piperazin-1-yl}-ethanone; |
| 49 | (4-Isopropyl-piperazin-1-yl)-[3-(4-thiazol-2-yl-piperazin-1-ylmethyl)-1H- |
| | indol-6-yl]-methanone; |
| 50 | 1-{4-[6-(4-Isopropyl-piperazine-1-carbonyl)-1H-indol-3-ylmethyl]- |
| | [1,4]diazepam-1-yl}-ethanone; |
| 51 | [3-(4-Benzyl-piperazin-1-ylmethyl)-1H-indol-6-yl]-(4-isopropyl-piperazin- |
| | 1-yl)-methanone; |
| 52 | [3-(4-Biphenyl-4-yl-piperazin-1-ylmethyl)-1H-indol-6-yl]-(4-isopropyl- |
| | piperazin-1-yl)-methanone; |
| 53 | [3-(4-Benzydryl-piperazin-1-ylmethyl)-1H-indol-6-yl]-(4-isopropyl- |
| | piperazin-1-yl)-methanone; |
| 54 | (4-Isopropyl-piperazin-1-yl)-[3-(4-methanesulfonyl-piperazin-1- |
| | ylmethyl)-1H-indol-6-yl]-methanone; |
| 55 | [3-(4-Benzyl-piperidin-1-ylmethyl)-1H-indol-6-yl]-(4-isopropyl-piperazin- |
| | 1-yl)-methanone; |
| 56 | (4-Isopropyl-piperazin-1-yl)-[3-(4-phenethyl-piperazin-1-ylmethyl)-1H- |
| | indol-6-yl]-methanone; |
| 57 | (4-Isopropyl-piperazin-1-yl)-[3-(4-phenyl-piperidin-1-ylmethyl)-1H-indol- |
| | 6-yl]-methanone; |
| 58 | (4-Isopropyl-piperazin-1-yl)-(3-pyrrolidin-1-ylmethyl-1H-indol-6-yl)- |
| | methanone; |
| 59 | (4-Isopropyl-piperazin-1-yl)-(3-[1,4]-oxazepan-4-ylmethyl-1H-indol-6- |
| | yl)-methanone; |
| 60 | N-{1-[6-(4-Isopropyl-piperazine-1-carbonyl)-1H-indol-3-ylmethyl]- |
| | pyrrolidin-3-yl}-N-methyl-acetamide; |
| 61 | (4-Isopropyl-piperazin-1-yl)-{3-[4-(morpholine-4-carbonyl)-piperazin-1- |
| | ylmethyl]-1H-indol-6-yl}-methanone; |
| 62 | (4-Isopropyl-piperazin-1-yl)-{3-[4-(pyridine-4-carbonyl)-piperazin-1- |
| | ylmethyl]-1H-indol-6-yl}-methanone; |
| 63 | [3-(4-Benzoyl-piperazin-1-ylmethyl)-1H-indol-6-yl]-(4-isopropyl- |
| | piperazin-1-yl)-methanone; |
| 64 | (4-Isopropyl-piperazin-1-yl)-[3-(4-pyridin-4-yl-piperazin-1-ylmethyl)-1H- |
| | indol-6-yl]-methanone; |
| 65 | [3-(4-Hydroxy-4-phenyl-piperidin-1-ylmethyl)-1H-indol-6-yl]-(4- |
| | isopropyl-piperazin-1-yl)-methanone; |
| 66 | (4-Isopropyl-piperazin-1-yl)-(3-thiomorpholin-4-ylmethyl-1H-indol-6-yl)- |
| | methanone; |
| 67 | (3-[1,4′]Bipiperidinyl-1′-ylmethyl-1H-indol-6-yl)-thiomorpholin-4-yl- |
| | methanone; |
| 68 | [3-(4-Cyclopentyl-piperazin-1-ylmethyl)-1H-indol-6-yl]-thiomorpholin-4- |
| | yl-methanone; |
| 69 | [3-(3-Dimethylamino-pyrrolidin-1-ylmethyl)-1H-indol-6-yl]-thiomorpholin- |
| | 4-yl-methanone; |
| 70 | [3-(4-Cyclobutyl-piperazin-1-ylmethyl)-1H-indol-6-yl]-thiomorpholin-4-yl- |
| | methanone; |
| 71 | [3-(4-Cyclobutyl-[1,4]diazepan-1-ylmethyl)-1H-indol-6-yl]-thiomorpholin- |
| | 4-yl-methanone; |
| 72 | [3-(4-Isopropyl-[1,4]diazepan-1-ylmethyl)-1H-indol-6-yl]-thiomorpholin- |
| | 4-yl-methanone; |
| 73 | (3-[1,4′]Bipiperidinyl-1′-ylmethyl-1H-indol-6-yl)-morpholin-4-yl- |
| | methanone; |
| 74 | [3-(4-Cyclopentyl-piperazin-1-ylmethyl)-1H-indol-6-yl]-morpholin-4-yl- |
| | methanone; |
| 75 | [3-(4-Cyclobutyl-piperazin-1-ylmethyl)-1H-indol-6-yl]-morpholin-4-yl- |
| | methanone; |
| 76 | [3-(4-Isopropyl-piperazin-1-ylmethyl)-1H-indol-6-yl]-morpholin-4-yl- |
| | methanone; |
| 77 | [3-(4-Cyclobutyl-[1,4]diazepan-1-ylmethyl)-1H-indol-6-yl]-morpholin-4- |
| | yl-methanone; |
| 78 | [3-(4-Isopropyl-[1,4]diazepan-1-ylmethyl)-1H-indol-6-yl]-morpholin-4-yl- |
| | methanone; |
| 79 | (3-[1,4′]Bipiperidinyl-1′-ylmethyl-1H-indol-6-yl)-(4-hydroxymethyl- |
| | piperidin-1-yl)-methanone; |
| 80 | [3-(4-Cyclopentyl-piperazin-1-ylmethy)l-1H-indol-6-yl]-(4- |
| | hydroxymethyl-piperidin-1-yl)-methanone; |
| 81 | [3-(4-Cyclobutyl-piperazin-1-ylmethy)l-1H-indol-6-yl]-(4-hydroxymethyl- |
| | piperidin-1-yl)-methanone; |
| 82 | (4-Hydroxymethyl-piperidin-1-yl)-[3-(4-isopropyl-piperazin-1-ylmethyl)- |
| | 1H-indol-6-yl]-methanone; |
| 83 | [3-(4-Cyclobutyl-[1,4]diazepan-1-ylmethyl)-1H-indol-6-yl]-(4- |
| | hydroxymethyl-piperidin-1-yl)-methanone; |
| 84 | (4-Hydroxymethyl-piperidin-1-yl)-[3-(4-isopropyl-[1,4]diazepan-1- |
| | ylmethyl)-1H-indol-6-yl]-methanone; |
| 85 | (3-[1,4′]Bipiperidinyl-1′-ylmethyl-1H-indol-6-yl)-(4-phenyl-piperidin-1-yl)- |
| | methanone; |
| 86 | [3-(4-Cyclopentyl-piperazin-ylmethyl)-1H-indol-6-yl]-(4-phenyl-piperidin- |
| | 1-yl)-methanone; |
| 87 | [3-(3-Dimethylamino-pyrrolidin-1-ylmethyl)-1H-indol-6-yl]-(4-phenyl- |
| | piperidin-1-yl)-methanone; |
| 88 | [3-(4-Dimethylamino-piperidin-1-ylmethyl)-1H-indol-6-yl]-(4-phenyl- |
| | piperidin-1-yl)-methanone; |
| 89 | [3-(4-Cyclobutyl-[1,4]diazepan-1-ylmethyl)-1H-indol-6-yl]-(4-phenyl- |
| | piperidin-1-yl)-methanone; |
| 90 | Azepan-1-yl-(3-[1,4′]bipiperidinyl-1′-ylmethyl-1H-indol-6-yl)-methanone; |
| 91 | Azepan-1-yl-[3-(4-cyclopentyl-piperazin-1-ylmethyl)-1H-indol-6-yl]- |
| | methanone; |
| 92 | Azepan-1-yl-[3-(3-dimethylamino-pyrrolidin-1-ylmethyl)-1H-indol-6-yl]- |
| | methanone; |
| 93 | Azepan-1-yl-[3-(4-cyclobutyl-piperazin-1-ylmethyl)-1H-indol-6-yl]- |
| | methanone; |
| 94 | [3-(3-Dimethylamino-pyrrolidin-1-ylmethyl)-1H-indol-6-yl]-(4- |
| | hydroxymethyl-piperidin-1-yl)-methanone; |
| 95 | Azepan-1-yl-[3-(4-isopropyl-[1,4]diazepan-1-ylmethyl)-1H-indol-6-yl]- |
| | methanone; |
| 96 | [3-(4-Dimethylamino-piperidin-1-ylmethyl)-1H-indol-6-yl]-(4- |
| | hydroxymethyl-piperidin-1-yl)-methanone; |
| 97 | [3-(4-Isopropyl-[1,4]diazepan-1-ylmethyl)-1H-indol-6-yl]-(4-phenyl- |
| | piperidin-1-yl)-methanone; |
| 98 | Azepan-1-yl-[3-(4-cyclobutyl-[1,4]diazepan-1-ylmethyl)-1H-indol-6-yl]- |
| | methanone; |
| 99 | (4-Benzyl-piperidin-1-yl)-[3-(4-cyclopentyl-piperazin-1-ylmethyl)-1H- |
| | indol-6-yl]-methanone; |
| 100 | (4-Benzyl-piperidin-1-yl)-[3-(4-dimethylamino-piperidin-1-ylmethyl)-1H- |
| | indol-6-yl]-methanone; |
| 101 | (4-Benzyl-piperidin-1-yl)-[3-dimethylamino-pyrrolidin-1-ylmethyl)-1H- |
| | indol-6-yl]-methanone; |
| 102 | (4-Isopropyl-piperazin-1-yl)-(3-piperidin-1-ylmethyl-benzo[b]thiophen-5- |
| | yl)-methanone; |
| 103 | (4-Cyclobutyl-piperazin-1-yl)-(3-piperidin-1-ylmethyl-benzo[b]thiophen- |
| | 5-yl)-methanone; |
| 104 | [5-(4-Isopropyl-piperazine-1-carbonyl)-benzo[b]thiophen-3-yl]-piperidin- |
| | 1-yl-methanone; |
| 105 | [5-(4-Cyclobutyl-piperazine-1-carbonyl)-benzo[b]thiophen-3-yl]- |
| | piperidin-1-yl-methanone; |
| 106 | (4-Benzyl-piperidin-1-yl)-(3-[1,4′]bipiperidinyl-1′-ylmethyl-1H-indol-6-yl)- |
| | methanone; |
| 107 | (4-Benzyl-piperidin-1-yl)-[3-(4-cyclobutyl-piperazin-1-ylmethyl)-1H- |
| | indol-6-yl]-methanone; and |
| 108 | (4-Benzyl-piperidin-1-yl)-[3-(4-cyclobutyl-[1,4]diazepan-1-ylmethyl)-1H- |
| | indol-6-yl]-methanone; |

and pharmaceutically acceptable salts thereof.

The invention includes also pharmaceutically acceptable salts of the compounds of Formula (I), preferably of those described above and of the specific compounds exemplified herein, and methods of treatment using such salts.

A “pharmaceutically acceptable salt” is intended to mean a salt of a free acid or base of a compound represented by Formula (I) that is non-toxic, biologically tolerable, or otherwise biologically suitable for administration to the subject. See, generally, S. M. Berge, et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002. Examples of pharmaceutically acceptable salts are those that are pharmacologically effective and suitable for contact with the tissues of patients without undue toxicity, irritation, or allergic response. A compound of Formula (I) may possess a sufficiently acidic group, a sufficiently basic group, or both types of functional groups, and accordingly react with a number of inorganic or organic bases, and inorganic and organic acids, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methyl benzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound of Formula (I) contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, any compatible mixture of acids such as those given as examples herein, and any other acid and mixture thereof that are regarded as equivalents or acceptable substitutes in light of the ordinary level of skill in this technology.

If the compound of Formula (I) is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein, and any other base and mixture thereof that are regarded as equivalents or acceptable substitutes in light of the ordinary level of skill in this technology. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of Formula (I), and treatment methods employing such pharmaceutically acceptable prodrugs. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of a compound of Formula (I). Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of Formula (I) as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of the compounds of Formula (I), which may also be used in the methods of the invention. A “pharmaceutically active metabolite” means a pharmacologically active product of metabolism in the body of a compound of Formula (I) or salt thereof. Prodrugs and active metabolites of a compound may be determined using routine techniques known or available in the art. See, e.g., Bertolini, et al., J. Med. Chem. 1997, 40, 2011-2016; Shan, et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites of the present invention are useful as modulators of the histamine H₃ receptor in the methods of the invention. As such modulators, the compounds may act as antagonists, agonists, or inverse agonists. “Modulators” include both inhibitors and activators, where “inhibitors” refer to compounds that decrease, prevent, inactivate, desensitize or down-regulate histamine H₃ receptor expression or activity, and “activators” are compounds that increase, activate, facilitate, sensitize, or up-regulate histamine H₃ receptor expression or activity.

The term “treat” or “treating” as used herein is intended to refer to administration of an active agent or composition of the invention to a subject for the purpose of effecting a therapeutic or prophylactic benefit through modulation of histamine H₃ receptor activity. Treating includes reversing, ameliorating, alleviating, inhibiting the progress of, lessening the severity of, or preventing a disease, disorder, or condition, or one or more symptoms of such disease, disorder or condition mediated through modulation of histamine H₃ receptor activity. The term “subject” refers to a mammalian patient in need of such treatment, such as a human.

Accordingly, the invention relates to methods of using the compounds described herein to treat subjects diagnosed with or suffering from a disease, disorder, or condition mediated by histamine H₃ receptor activity, such as: cognitive disorders, sleep disorders, psychiatric disorders, and other disorders. Symptoms or disease states are intended to be included within the scope of “medical conditions, disorders, or diseases.”

Cognitive disorders include, for example, dementia, Alzheimer's disease (Panula, P. et al., Soc. Neurosci. Abstr. 1995, 21, 1977), cognitive dysfunction, mild cognitive impairment (pre-dementia), attention deficit hyperactivity disorders (ADHD), attention-deficit disorders, and learning and memory disorders (Barnes, J. C. et al., Soc. Neurosci. Abstr. 1993, 19, 1813). Learning and memory disorders include, for example, learning impairment, memory impairment, age-related cognitive decline, and memory loss. H₃ antagonists have been shown to improve memory in a variety of memory tests, including the elevated plus maze in mice (Miyazaki, S. et al. Life Sci. 1995, 57(23), 2137-2144), a two-trial place recognition task (Orsetti, M. et al. Behav. Brain Res. 2001, 124(2), 235-242), the passive avoidance test in mice (Miyazaki, S. et al. Meth. Find. Exp. Clin. Pharmacol. 1995, 17(10), 653-658) and the radial maze in rats (Chen, Z. Acta Pharmacol. Sin. 2000, 21(10), 905-910). Also, in the spontaneously hypertensive rat, an animal model for the learning impairments in attention-deficit disorders, H₃ antagonists were shown to improve memory (Fox, G. B. et al. Behav. Brain Res. 2002, 131(1-2), 151-161).

Sleep disorders include, for example, insomnia, disturbed sleep, narcolepsy (with or without associated cataplexy), cataplexy, disorders of sleep/wake homeostasis, idiopathic somnolence, excessive daytime sleepiness (EDS), circadian rhythm disorders, fatigue, lethargy, jet lag (phase delay), and REM-behavioral disorder. Fatigue and/or sleep impairment may be caused by or associated with various sources, such as, for example, sleep apnea, perimenopausal hormonal shifts, Parkinson's disease, multiple sclerosis (MS), depression, chemotherapy, or shift work schedules.

Psychiatric disorders include, for example, schizophrenia (Schlicker, E. and Marr, I., Naunyn-Schmiedeberg's Arch. Pharmacol. 1996, 353, 290-294), including cognitive deficits and negative symptoms associated with schizophrenia, bipolar disorders, manic disorders, depression (Lamberti, C. et al. Br. J. Pharmacol. 1998, 123(7), 1331-1336; Perez-Garcia, C. et al. Psychopharmacology 1999, 142(2), 215-220) (Also see: Stark, H. et al., Drugs Future 1996, 21(5), 507-520; and Leurs, R. et al., Prog. Drug Res. 1995, 45, 107-165 and references cited therein.), including bipolar depression, obsessive-compulsive disorder, and post-traumatic stress disorder.

Other disorders include, for example, motion sickness, vertigo (e.g. vertigo or benign postural vertigo), tinitus, epilepsy (Yokoyama, H. et al., Eur. J. Pharmacol. 1993, 234, 129-133), migraine, neurogenic inflammation, neuropathic pain, Down Syndrome, seizures, eating disorders (Machidori, H. et al., Brain Res. 1992, 590, 180-186), obesity, substance abuse disorders, movement disorders (e.g. restless legs syndrome), and eye-related disorders (e.g. macular degeneration and retinitis pigmentosis).

Particularly, as modulators of the histamine H₃ receptor, the compounds of the present invention are useful in the treatment or prevention of depression, disturbed sleep, narcolepsy, fatigue, lethargy, cognitive impairment, memory impairment, memory loss, learning impairment, attention-deficit disorders, and eating disorders.

In treatment methods according to the invention, an effective amount of at least one compound according to the invention is administered to a subject suffering from or diagnosed as having such a disease, disorder, or condition. An “effective amount” means an amount or dose sufficient to generally bring about the desired therapeutic or prophylactic benefit in patients in need of such treatment for the designated disease, disorder, or condition. Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the compounds of the invention may be used in combination with additional active ingredients in the treatment of the above conditions. In an exemplary embodiment, additional active ingredients are those that are known or discovered to be effective in the treatment of conditions, disorders, or diseases mediated by histamine H₃ receptor activity or that are active against another target associated with the particular condition, disorder, or disease, such as H₁ receptor antagonists, H₂ receptor antagonists, H₃ receptor antagonists, topiramate (TOPAMAX™), and neurotransmitter modulators such as serotonin-norepinephrine reuptake inhibitors, selective serotonin reuptake inhibitors (SSRIs), noradrenergic reuptake inhibitors, non-selective serotonin re-uptake inhibitors (NSSRIs), acetylcholinesterase inhibitors (such as tetrahydroaminoacridine, Donepezil (ARICEPT™), Rivastigmine, or Galantamine (REMINYL™)), or modafinil. The combination may serve to increase efficacy (e.g., by including in the combination a compound potentiating the potency or effectiveness of a compound according to the invention), decrease one or more side effects, or decrease the required dose of the compound according to the invention.

More particularly, compounds of the invention in combination with modafinil are useful for the treatment of narcolepsy, excessive daytime sleepiness (EDS), Alzheimer's disease, depression, attention-deficit disorders, MS-related fatigue, post-anesthesia grogginess, cognitive impairment, schizophrenia, spasticity associated with cerebral palsy, age-related memory decline, idiopathic somnolence, or jet-lag. Preferably, the combination method employs doses of modafinil in the range of about 20 to 300 mg per dose.

In another embodiment, compounds of the invention in combination with topiramate are useful for the treatment of obesity. Preferably, the combination method employs doses of modafinil in the range of about 20 to 300 mg per dose.

The compounds of the invention are used, alone or in combination with one or more other active ingredients, to formulate pharmaceutical compositions of the invention. A pharmaceutical composition of the invention comprises: (a) an effective amount of a compound of Formula (I), or a pharmaceutically acceptable salt, pharmaceutically acceptable prodrug, or pharmaceutically active metabolite thereof; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of a compound of the invention and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the compounds of the invention may be prepared using suitable pharmaceutical excipients and compounding techniques now or later known or available to those skilled in the art. The compositions may be administered in the inventive methods by oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.01 to about 100 mg/kg daily, or from about 0.05 to about 35 mg/kg daily, or from about 0.1 to about 10 mg/kg daily.

Oral tablets may include a compound according to the invention mixed with pharmaceutically acceptable excipients such as inert diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms will be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 μg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery.

Compounds of the invention may alternatively be administered in methods of this invention by inhalation, via the nasal or oral routes, e.g., in a spray formulation also containing a suitable carrier.

Exemplary compounds useful in methods of the invention will now be described by reference to the illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables are as defined above in reference to Formula (I). Reactions may be performed between the melting point and the reflux temperature of the solvent, and preferably between 0° C. and the reflux temperature of the solvent.

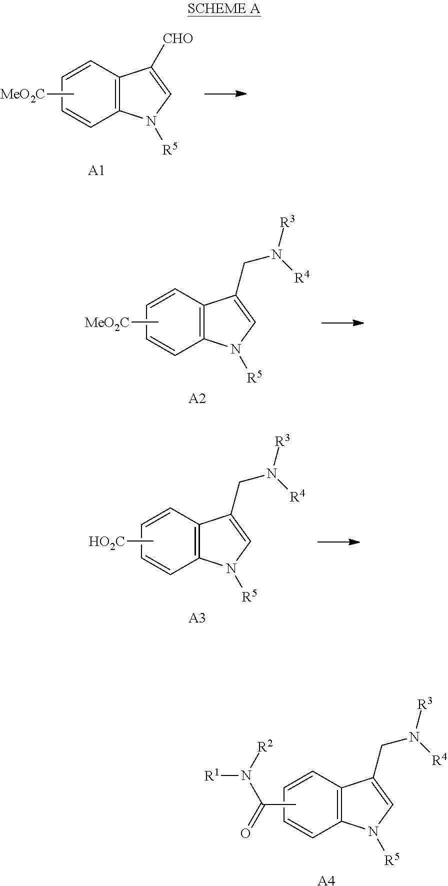

Certain embodiments of compounds of Formula (I), such as amides A4, are prepared from indoles A1 (which are commercially available or known in the art) as shown in Scheme A. Reductive amination of aldehydes A2 with amines HNR³R⁴ provides amines A2. Preferred conditions include treatment with a reducing agent such as NaBH(OAc)₃ or NaCNBH₃ in a solvent such as 1,2-dichloroethane (DCE), with optional additives such as acetic acid or a Lewis acid (e.g. ZnCl₂). Hydrolysis of the ester moiety under general conditions provides acids A3 or their corresponding salts. Coupling of acids A3 with suitable amines HNR³R⁴ gives amides A4. Preferred reaction conditions include, for example: 1) treatment with 1-(3-dimethylaminopropyl)-3-ethylcarbodiimide (EDC) and 1-hydroxybenzotriazole (HOBt) in a solvent such as N,N-dimethylformamide (DMF); or 2) formation of the mixed anhydride and subsequent treatment with amines HNR³R⁴. One skilled in the art will recognize that where R₅ is —SO₂Me, it is prepared from compounds where R⁵ is —H by reaction with methanesulfonyl chloride in the presence of a suitable base, such as triethylamine, in a solvent such as dichloromethane (DCM).

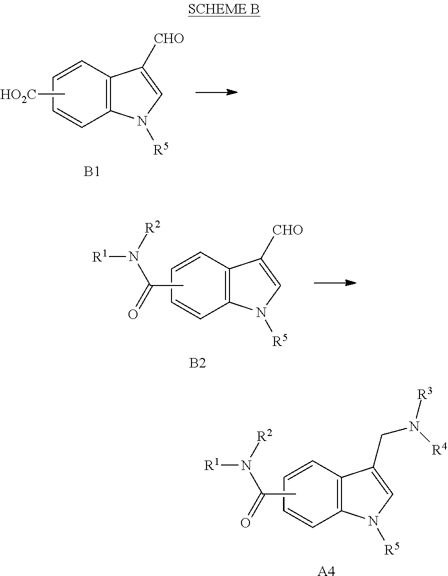

Compounds A4 are also prepared according to Scheme B. Indole acids B1 are converted to amides B2 by reaction with amines HNR¹R² as described in Scheme A. Reductive amination with amines HNR³R⁴ as described in Scheme A provides amides A4.

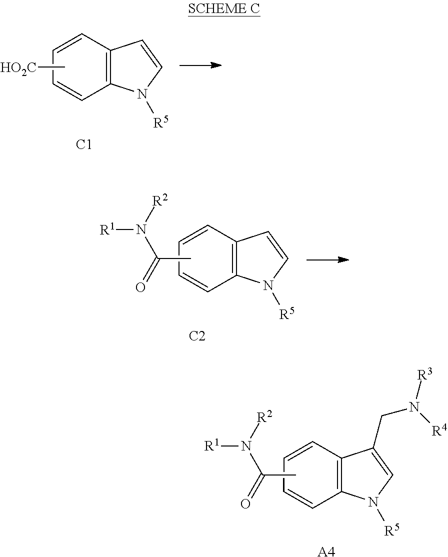

Compounds A4, where R⁵ is —H or methyl, are also prepared according to Scheme C. Indole acids C1 are converted to amides C2 by reaction with amines HNR¹R² as described in Scheme A. Mannich reaction with amines HNR³R⁴ in the presence of formaldehyde yields compounds A4. One skilled in the art will recognize that Mannich reactions are alternatively performed on the corresponding esters of indole acids C1. Conversion of the esters to amides and reductive amination as described in the preceding schemes yields compounds A4.

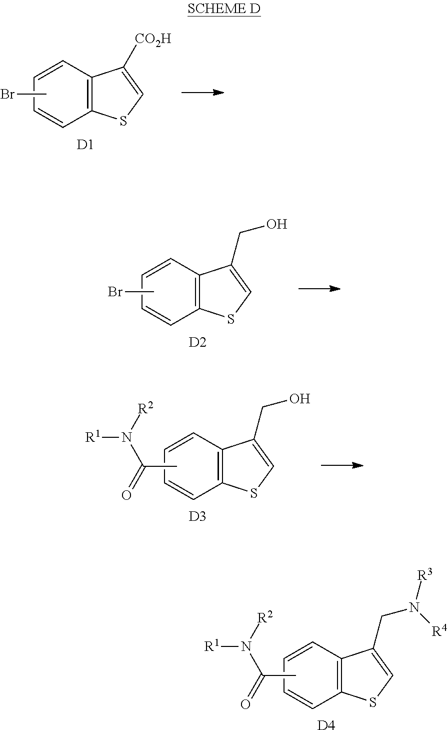

Certain embodiments of Formula (I), such as amides D4, are prepared according to Scheme D. Benzothiophene acids D1 are reduced to the corresponding alcohols D2. Coupling with amines HNR¹R² as described in Scheme A provide amides D3. Oxidation to the corresponding aldehydes (not shown) followed by reductive amination with amines HNR³R⁴ as described in Scheme A give compounds D4.

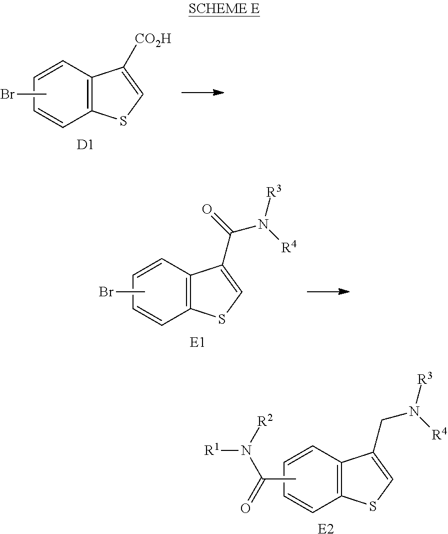

Certain embodiments of Formula (I), such as amides E2, are prepared according to Scheme E. Benzothiophene acids D1 are coupled with amines HNR³R⁴ as described in Scheme A to give amides E1. Transition metal-catalyzed reaction of bromides E1 with amines HNR¹R² and a CO equivalent, such as CO gas or Mo(CO)₆, in the presence of a suitable palladium (II) catalyst and a suitable base (such as 1,8-diazabicyclo[5.4.0]undec-7-ene (DBU)), and optional additives such as t-BuPHBF₄⁺, provides compounds E2.

Those skilled in the art will recognize that several of the chemical transformations described above may be performed in a different order than that depicted in the above Schemes.

Compounds of Formula (I) may be converted to their corresponding salts using methods known to those skilled in the art. For example, amines of Formula (I) may be treated with trifluoroacetic acid (TFA), HCl, maleic acid, or citric acid in a solvent such as diethyl ether (Et₂O), CH₂Cl₂, tetrahydrofuran (THF), or methanol (MeOH) to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation methods known to one skilled in the art, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using conventional methods such as chromatography or crystallization.

The following examples are provided to further illustrate the invention and various preferred embodiments.

### EXAMPLES

### Chemistry

In preparing the compounds described in the examples below and obtaining the corresponding analytical data, the following experimental and analytical protocols were followed unless otherwise indicated.

Unless otherwise specified, reaction mixtures were magnetically stirred at room temperature (rt) under a N_{2(g)} atmosphere. Where solutions were “dried,” they were generally dried over a drying agent such as Na₂SO₄ or MgSO₄. Where mixtures, solutions, and extracts were “concentrated”, they were typically concentrated on a rotary evaporator under reduced pressure.

Normal-phase flash column chromatography (FCC) was performed on silica gel (SiO₂) eluting with 2 M NH₃ in MeOH/DCM, unless otherwise noted.

Reverse phase high performance liquid chromatography (HPLC) was performed on a Hewlett Packard HPLC Series 1100, with a Phenomenex Luna C18 (5 μm, 4.6×150 mm) column. Detection was done at λ=230, 254 and 280 nm. The gradient was 10 to 99% acetonitrile/H₂O (0.05% trifluoroacetic acid (TFA)) over 5.0 min with a flow rate of 1 mL/min (acidic conditions). Alternatively, HPLC was performed on a Dionex APS2000 LC/MS with a Phenomenex Gemini C18 (5 μm, 30×100 mm) column, and a gradient of 5 to 100% acetonitrile/H₂O (20 mM NH₄OH) over 16.3 min, and a flow rate of 30 mL/min (basic conditions). Retention times (Rₜ) are provided in minutes.

Mass spectra (MS) were obtained on an Agilent series 1100 MSD using electrospray ionization (ESI) in positive mode unless otherwise indicated. Calculated (calcd.) mass corresponds to the exact mass.

Nuclear magnetic resonance (NMR) spectra were obtained on Bruker model DRX spectrometers. The format of the ¹H NMR data below is: chemical shift in ppm downfield of the tetramethylsilane reference (multiplicity, coupling constant J in Hz, integration).

Chemical names were generated using ChemDraw Version 6.0.2 (CambridgeSoft, Cambridge, Mass.).

### Example 1

### (4-Isopropyl-piperazin-1-yl)-(1-methyl-3-morpholin-4-ylmethyl-1H-indol-6-yl)-methanone

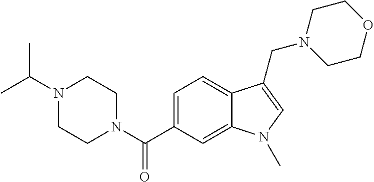

Step A: 3-Morpholin-4-ylmethyl-1H-indole-6-carboxylic acid methyl ester. To a solution of methyl 3-formyl-indole-6-carboxylic acid (1.0 g, 5.0 mmol) and morpholine (470 mg, 5 mmol) in 1,2-dichloroethane (50 mL) was added NaBH(OAc)₃ (2.6 g, 12 mmol). After 24 h, the solution was concentrated and the resulting residue was partitioned between DCM and 1 N NaOH (50 mL). The organic layer was washed with brine (50 mL), dried, and concentrated to provide 1.13 g (84%) of the title compound as a white solid. MS (ESI): mass calcd. for C₁₅H₁₈N₂O₃, 274.13; m/z found, 275.1 [M+H]⁺. ¹H NMR (CDCl₃): 8.40 (br s, 1H), 8.14 (d, J=0.5, 1H), 7.83 (q, J=8.4, 1.4, 1H), 7.79 (d, J=8.4, 1H), 7.32 (d, J=2.3, 1H), 3.95 (s, 3H), 3.73-3.71 (m, 6H), 2.51 (s, 4H).

Step B: 1-Methyl-3-morpholin-4-ylmethyl-1H-indole-6-carboxylic acid methyl ester. To a 0° C. solution of 3-morpholin-4-ylmethyl-1H-indole-6-carboxylic acid methyl ester (170 mg, 6 mmol) in N,N-dimethylformamide (DMF; 6 mL) was added NaH (40 mg, 9 mmol). The suspension was stirred for 15 min at 0° C. and then was warmed to rt over 15 min. The suspension was cooled to 0° C. and treated with MeI (132 mg, 9 mmol) and stirred at 0° C. for 2 h. The suspension was partitioned between ethyl acetate (EtOAc) and brine (50 mL). The organic layer was washed with brine (50 mL), dried, and concentrated to provide 0.16 g (92%) of the title compound as an amber oil. MS (ESI): mass calcd. for C₁₆H₂₀N₂O₃, 288.15; m/z found, 289.1 [M+H]⁺. ¹H NMR (CDCl₃): 8.09 (d, J=0.7, 1H), 7.82 (q, J=8.4, 1.4, 1H), 7.76-7.45 (m, 1H), 7.18 (s, 1H), 3.96 (s, 3H), 3.85 (s, 3H), 3.73-3.70 (m, 6H), 2.50 (s, 4H).

Step C: Potassium 1-methyl-3-morpholin-4-ylmethyl-1H-indole-6-carboxylate. To a solution of 1-methyl-3-morpholin-4-ylmethyl-1H-indole-6-carboxylic acid methyl ester (490 mg, 2 mmol) in i-PrOH (17 mL) was added 2 N KOH (1.0 mL, 2 mmol). The reaction mixture was heated at 55° C. for 24 h. The solution was concentrated to provide 0.52 g (100%) of the title compound as a white solid, which was used in the next step without further purification.

Step D: (4-Isopropyl-piperazin-1-yl)-(1-methyl-3-morpholin-4-ylmethyl-1H-indol-6-yl)-methanone. To a suspension of potassium 1-methyl-3-morpholin-4-ylmethyl-1H-indole-6-carboxylate (175 mg, 0.56 mmol) 1-isopropyl-piperazine (72 mg, 0.56 mmol), 1-hydroxybenzotriazole (HOBt; 130 mg, 0.84 mmol) in DMF (2.8 mL) was added 1-(3-dimethylaminopropyl)-3-ethylcarbodiimide hydrochloride (EDC; 162 mg, 0.84 mmol). After 24 h, the reaction mixture was partitioned between EtOAc and 1 N NaOH (25 mL). The organic layer was washed with brine, dried and concentrated. The resulting residue was purified by FCC to provide 117 mg (54%) of the title compound. LC/MS: Rₜ=1.51. MS (ESI): mass calcd. for C₂₂H₃₂N₄O₂, 384.53; m/z found, 385.3 [M+H]⁺. ¹H NMR (CDCl₃): 7.72 (d, J=8.2, 1H), 7.47 (s, 1H), 7.14 (q, J=8.2, 1.3, 1H), 7.09 (s, 1H), 3.93-3.56 (m, 8H), 3.80 (s, 2H), 3.56 (s, 3H), 2.72 (h, J=6.6, 1H), 2.60-2.45 (m, 8H), 1.07 (d, J=6.6, 6H).

The compounds in Example 2-Example 3 were prepared using methods analogous to those described for Example 1.

### Example 2

### (4-Cyclopropyl-piperazin-1-yl)-(1-methyl-3-morpholin-4-ylmethyl-1H-indol-6-yl)-methanone

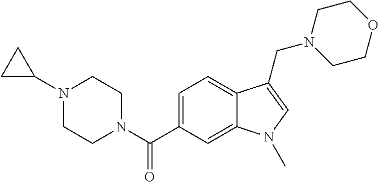

LC/MS: Rₜ=1.51. MS (ESI): mass calcd. for C₂₂H₃₀N₄O₂, 382.51; m/z found, 383.3 [M+H]⁺. ¹H NMR (CDCl₃): 7.74 (d, J=8.0, 1H), 7.47 (s, 1H), 7.14 (dd, J=8.0, 1.5, 1H), 7.09-7.08 (m, 1H), 3.76-3.69 (m, 10H), 3.62-3.46 (m, 2H), 3.12-3.07 (m, 1H), 2.86-2.61 (m, 4H), 2.38 (s, 3H), 1.68-1.65 (m, 2H), 0.51-0.42 (m, 4H).

### Example 3

### (4-Cyclobutyl-piperazin-1-yl)-(1-methyl-3-morpholin-4-ylmethyl-1H-indol-6-yl)-methanone

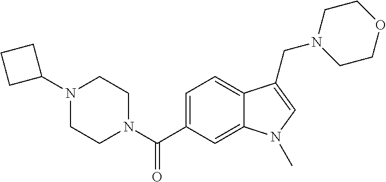

LC/MS: Rₜ=1.53. MS (ESI): mass calcd. for C₂₃H₃₂N₄O₂, 396.54; m/z found, 397.3 [M+H]⁺. ¹H NMR (CDCl₃): 7.72 (d, J=8.0, 1H), 7.46 (s, 1H), 7.13 (dd, J=8.0, 1.5, 1H), 7.09 (s, 1H), 3.83-3.55 (m, 11H), 2.77 (p, J=7.5, 1H), 2.49 (br s, 3H), 2.46-2.29 (m, 5H), 2.08-2.03 (m, 2H), 1.92-1.87 (m, 2H), 1.76-1.70 (m, 4H).

The compounds in Example 4-Example 19 were prepared using methods analogous to those described for Example 1, Steps A, C, and D.

### Example 4

### (4-Isopropyl-piperazin-1-yl)-(3-morpholin-4-ylmethyl-1H-indol-6-yl)-methanone

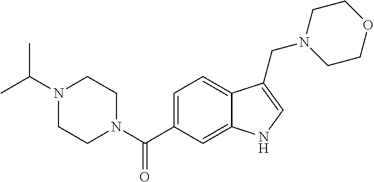

LC/MS: Rₜ=1.45. MS (ESI): mass calcd. for O₂₁H₃₀N₄O₂, 370.50; m/z found, 371.3 [M+H]⁺. ¹H NMR (CDCl₃): 8.59 (br s, 1H), 7.75 (d, J=8.2, 1H), 7.51 (d, J=0.5, 1H), 7.21 (d, J=1.4, 1H), 7.16 (dd, J=8.2, 1.4, 1H), 3.90-3.50 (m, 10H), 2.73 (h, J=6.6, 1H), 2.62-2.48 (m, 8H), 1.07 (d, J=6.6, 6H).

### Example 5

### (4-Cyclopropyl-piperazin-1-yl)-(3-morpholin-4-ylmethyl-1H-indol-6-yl)-methanone

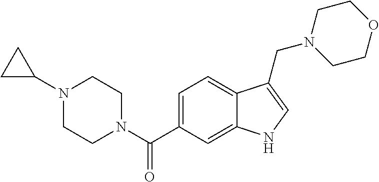

LC/MS: Rₜ=1.44. MS (ESI): mass calcd. for C₂₁H₂₈N₄O₂, 368.48; m/z found, 369.2 [M+H]⁺. ¹H NMR (CDCl₃): 8.52 (br s, 1H), 7.76 (dd, J=8.2, 3.8, 1H), 7.51 (s, 1H), 7.21 (d, J=2.3, 1H), 7.17 (dd, J=8.2, 1.3, 1H), 3.76-3.50 (m, 10H), 2.65-2.50 (m, 8H), 1.66 (p, J=3.4, 1H), 0.51-0.49 (m, 4H).

### Example 6

### (4-Cyclobutyl-piperazin-1-yl)-(3-morpholin-4-ylmethyl-1H-indol-6-yl)-methanone

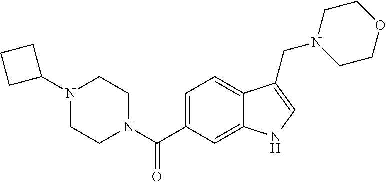

LC/MS: Rₜ=1.48. MS (ESI): mass calcd. for C₂₂H₃₀N₄O₂, 382.51; m/z found, 383.3 [M+H]⁺. ¹H NMR (CDCl₃): 8.60 (br s, 1H), 7.75 (d, J=8.3, 1H), 7.51 (s, 1H), 7.21 (d, J=1.7, 1H), 7.15 (dd, J=8.3, 1.7, 1H), 3.90-3.50 (m, 8H), 2.76 (p, J=7.9, 1H), 2.51-2.29 (m, 8H), 2.08-2.03 (m, 2H), 1.91-1.87 (m, 2H), 1.77-1.70 (m, 4H).

### Example 7

### (4-Isopropyl-[1,4]diazepan-1-yl)-(3-morpholin-4-ylmethyl-1H-indol-6-yl)-methanone

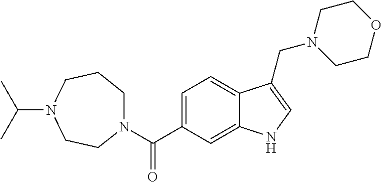

LC/MS: Rₜ=1.52. MS (ESI): mass calcd. for C₂₂H₃₂N₄O₂, 384.53; m/z found, 385.3 [M+H]⁺. ¹H NMR (CDCl₃): 9.00 (br s, 1H), 7.71 (d, J=8.4, 1H), 7.45 (s, 1H), 7.16-7.12 (m, 2H), 3.80 (s, 2H), 3.71-3.68 (m, 6H), 3.52-3.49 (m, 2H), 2.96-2.83 (m, 2H), 2.71-2.59 (m, 1H), 2.62-2.60 (m, 2H), 2.48 (s, 4H), 1.95 (s, 1H), 1.75 (s, 1H), 1.05-0.97 (m, 6H).

### Example 8

### (4-Cyclopropyl-[1,4]diazepan-1-yl)-(3-morpholin-4-ylmethyl-1H-indol-6-yl)-methanone

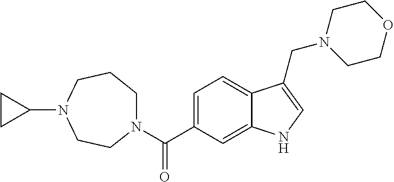

LC/MS: Rₜ=1.51. MS (ESI): mass calcd. for C₂₂H₃₀N₄O₂, 382.51; m/z found, 383.3 [M+H]⁺. ¹H NMR (CDCl₃): 9.19 (br s, 1H), 7.71 (d, J=8.4, 1H), 7.44 (s, 1H), 7.14-7.10 (m, 2H), 3.79 (br s, 2H), 3.71-3.67 (m, 6H), 3.53-3.51 (m, 2H), 2.99 (br s, 1H), 2.87-2.77 (m, 3H), 2.48 (br s, 3H), 1.97-1.77 (m, 4H), 0.50-036 (m, 4H).

### Example 9

### (4-Cyclobutyl-[1,4]diazepan-1-yl)-(3-morpholin-4-ylmethyl-1H-indol-6-yl)-methanone

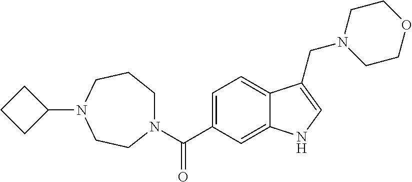

LC/MS: Rₜ=1.54. MS (ESI): mass calcd. for C₂₃H₃₂N₄O₂, 396.54; m/z found, 397.3 [M+H]⁺. ¹H NMR (CDCl₃): 9.14 (br s, 1H), 7.71 (d, J=8.1, 1H), 7.44 (s, 1H), 7.15-7.11 (m, 2H), 3.81 (br s, 2H), 3.71-3.67 (m, 6H), 2.58-2.51 (m, 2H), 2.88 (p, J=7.8, 1H), 2.66 (br s, 1H), 2.53-2.44 (m, 7H), 2.08-1.99 (m, 3H), 1.89-1.76 (m, 3H), 1.71-1.62 (m, 2H).

### Example 10

### 3-Morpholin-4-ylmethyl-1H-indole-6-carboxylic acid (pyridin-3-ylmethyl)-amide

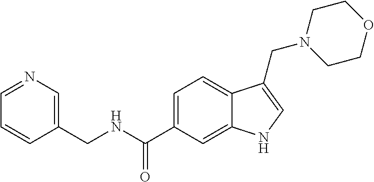

LC/MS: Rₜ=3.27. MS (ESI): mass calcd. for C₂₀H₂₂N₄O₂, 350.42; m/z found, 351.2 [M+H]⁺. ¹H NMR (CDCl₃): 9.99 (br s, 1H), 8.54 (br s, 1H), 8.41 (br s, 1H), 7.90 (br s, 1H), 7.68 (d, J=8.3, 1H), 7.60 (d, J=6.5, 1H), 7.49 (d, J=8.3, 2H), 7.20 (br s, 1H), 7.14 (br s, 1H), 4.57 (br s, 2H), 3.65 (br s, 6H), 2.44 (br s, 4H).

### Example 11

### 3-Morpholin-4-ylmethyl-1H-indole-6-carboxylic acid (pyridin-4-ylmethyl)-amide

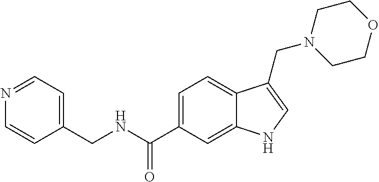

LC/MS: Rₜ=3.25. MS (ESI): mass calcd. for C₂₀H₂₂N₄O₂, 350.42; m/z found, 351.2 [M+H]⁺. ¹H NMR (CDCl₃): 9.85 (br s, 1H), 8.40 (br s, 2H), 7.94 (br s, 1H), 7.71 (d, J=7.8, 1H), 7.52 (d, J=7.8, 1H), 7.43-7.41 (m, 1H), 7.19 (br s, 1H), 7.12 (br s, 2H), 4.55 (br s, 2H), 3.66 (br s, 6H), 2.45 (br s, 4H).

### Example 12

### 3-Morpholin-4-ylmethyl-1H-indole-6-carboxylic acid (3-methyl-pyridin-2-ylmethyl)-amide

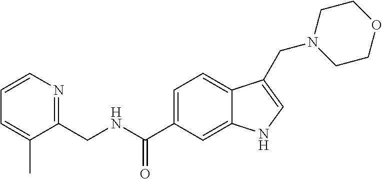

LC/MS: Rₜ=3.40. MS (ESI): mass calcd. for O₂₁H₂₄N₄O₂, 364.45; m/z found, 365.2 [M+H]⁺. ¹H NMR (CDCl₃): 9.74 (br s, 1H), 8.40 (d, J=4.0, 1H), 8.32 (t, J=4.0, 1H), 8.12 (br s, 1H), 7.76 (d, J=7.5, 1H), 7.62 (dd, J=10.0, 2.3, 1H), 7.47 (d, J=7.5, 1H), 7.24 (d, J=2.3, 1H), 7.14 (dd, J=7.5, 4.5, 1H), 4.72 (d, J=4.5, 2H), 3.69 (br s, 6H), 2.48 (br s, 4H), 2.31 (s, 3H).

### Example 13

### (3-Morpholin-4-ylmethyl-1H-indol-6-yl)-(3,4,6,7-tetrahydro-imidazo[4,5-d]pyridin-5-yl)-methanone

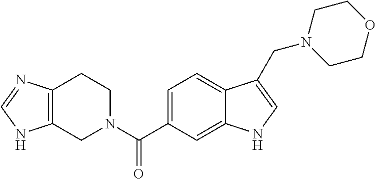

LC/MS: Rₜ=3.59. MS (ESI): mass calcd. for C₂₀H₂₃N₅O₂, 365.44; m/z found, 366.2 [M+H]⁺. ¹H NMR (CDCl₃): 9.40-9.70 (br s, 1H), 7.71 (d, J=7.3, 1H), 7.40 (br s, 1H), 7.15 (d, J=7.3, 2H), 7.40-7.16 (m, 1H), 4.80-4.50 (m, 2H), 4.10-3.60 (m, 8H), 2.76 (br s, 2H), 2.49 (br s, 4H).

### Example 14

### 1-[1-(3-Morpholin-4-ylmethyl-1H-indole-6-carbonyl)-piperidin-4-yl]-pyrrolidin-2-one

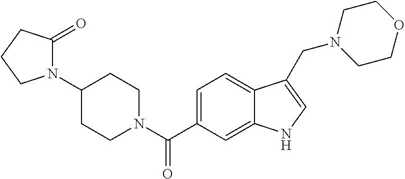

LC/MS: Rₜ=3.81. MS (ESI): mass calcd. for C₂₃H₃₀N₄O₃, 410.52; m/z found, 411.3 [M+H]⁺. ¹H NMR (CDCl₃): 9.17 (br 5, 1H), 7.73-7.71 (m, 1H), 7.49 (5, 1H), 7.19-7.16 (m, 1H), 7.13-7.11 (m, 1H), 4.83 (br 5, 1H), 4.25-4.20 (m, 1H), 4.21-4.03 (br s, 1H), 3.69 (br s, 6H), 3.35-3.23 (m, 2H), 3.00-2.97 (br s, 2H), 2.48 (br s, 4H), 2.40 (t, J=8.0, 2H), 2.05-2.00 (m, 2H), 1.66 (br s, 4H).

### Example 15

### 3-Piperidin-1-ylmethyl-1H-indole-6-carboxylic acid (pyridin-3-ylmethyl)-amide

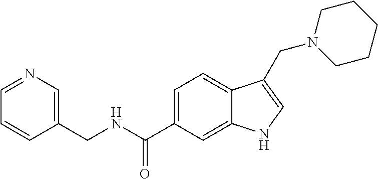

LC/MS: Rₜ=3.45. MS (ESI): mass calcd. for C₂₁H₂₄N₄O, 348.45; m/z found, 349.2 [M+H]⁺. ¹H NMR (CDCl₃): 10.51 (br s, 1H), 8.56 (br s, 1H), 8.36 (br s, 1H), 7.86 (5, 1H), 7.77 (br s, 1H), 7.60 (d, J=7.0, 1H), 7.55 (d, J=8.5, 1H), 7.49 (d, J=8.5 1H), 7.11 (br s, 1H), 4.57 (br s, 2H), 3.70 (5, 2H), 3.48 (br s, 4H), 1.55 (br s, 4H), 1.37 (br s, 2H).

### Example 16

### 3-Piperidin-1-ylmethyl-1H-indole-6-carboxylic acid (pyridin-4-ylmethyl)-amide

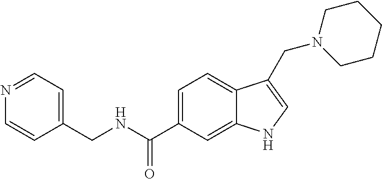

LC/MS: Rₜ=3.43. MS (ESI): mass calcd. for C₂₁H₂₄N₄O, 348.45; m/z found, 349.2 [M+H]⁺. ¹H NMR (CDCl₃): 10.17 (br s, 1H), 9.35-8.33 (m, 2H), 7.82 (s, 1H), 7.63 (d, J=8.0, 1H), 7.53-7.48 (m, 2H), 7.15 (s, 1H), 7.07-7.04 (m, 2H), 4.49-4.47 (m, 2H), 3.64 (br s, 2H), 2.42 (br s, 4H), 1.52-1.50 (m, 4H), 1.37 (s, 2H).

### Example 17

### 3-Piperidin-1-ylmethyl-1H-indole-6-carboxylic acid (3-methyl-pyridin-2-ylmethyl)-amide

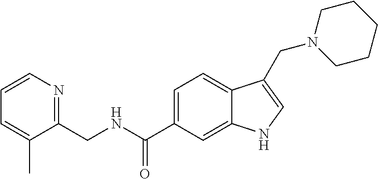

LC/MS: Rₜ=3.57. MS (ESI): mass calcd. for C₂₂H₂₆N₄O, 362.48; m/z found, 363.2 [M+H]⁺. ¹H NMR (CDCl₃): 10.28 (br s, 1H), 8.36 (br s, 1H), 8.30 (br s, 1H), 8.00 (br s, 1H), 7.68 (d, J=8.0, 1H), 7.58 (d, J=8.0, 1H), 7.44-7.42 (m, 1H), 7.22 (br s, 1H), 7.11-7.08 (m, 1H), 4.69 (br s, 2H), 3.69 (s, 2H), 2.47 (br s, 4H), 2.29-2.28 (m, 3H), 1.56 (br s, 4H), 1.39 (br s, 2H).

### Example 18

### (3-Piperidin-1-ylmethyl-1H-indol-6-yl)-(3,4,6,7-tetrahydro-imidazo[4,5-c]pyridin-5-yl)-methanone

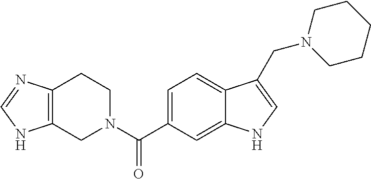

LC/MS: Rₜ=3.81. MS (ESI): mass calcd. for C₂₁H₂₅N₅O, 363.47; m/z found, 364.2 [M+H]⁺. ¹H NMR (CDCl₃): 11.96 (br s, 1H), 10.30 (br s, 1H), 7.61 (d, J=7.5, 1H), 7.35 (s, 1H), 7.14 (s, 1H), 7.09 (d, J=7.5, 1H), 4.69-4.45 (m, 2H), 3.91-3.57 (m, 4H), 2.62-2.51 (m, 6H), 1.58 (br s, 4H), 1.40 (br s, 2H).

### Example 19

### 1-[1-(3-Piperidin-1-ylmethyl-1H-indole-6-carbonyl)-piperidin-4-yl]-pyrrolidin-2-one

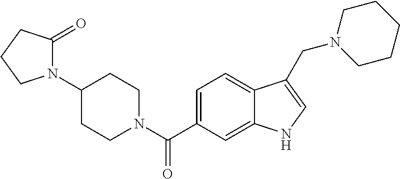

LC/MS: Rₜ=4.01. MS (ESI): mass calcd. for C₂₄H₃₂N₄O₂, 408.55; m/z found, 409.3 [M+H]⁺. ¹H NMR (CDCl₃): 9.99 (br s, 1H), 7.63 (d, J=8.0, 1H), 7.43 (s, 1H), 7.12 (s, 1H), 7.07 (d, J=8.0, 1H), 4.78 (br s, 1H), 4.21-4.17 (m, 1H), 4.02-3.96 (br s, 1H), 3.67 (br s, 2H), 3.33-3.27 (m, 2H), 3.10-2.86 (br s, 2H), 2.44 (br s, 4H), 2.37 (t, J=8.0, 2H), 1.99-1.96 (m, 2H), 1.70-1.53 (br m, 8H), 1.34 (br s, 2H).

### Example 20

### (4-Isopropyl-piperazin-1-yl)-(3-morpholin-4-ylmethyl-1H-indol-7-yl)-methanone

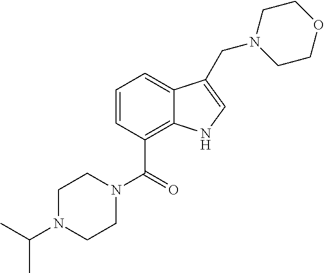

Step A: 4-(3-Formyl-1H-indole-7-carbonyl)-piperazine-1-carboxylic acid tert-butyl ester. To a solution of 3-formyl-indole-7-carboxylic acid (1.0 g, 5.3 mmol) and piperazine-1-carboxylic acid tert-butyl ester (0.98 g, 5.3 mmol) in DMF (26 mL) was added HOBt (1.23 g, 7.9 mmol) and EDC (1.50 g, 7.9 mmol). After 24 h, the reaction mixture was partitioned between EtOAc and 1 N NaOH (25 mL). The organic layer was washed with brine (50 mL), dried, and concentrated to provide 0.64 g (34%) of the title compound as an amber oil. MS (ESI): mass calcd. for C₁₉H₂₃N₃O₄, 357.17; m/z found, 358.2 [M+H]⁺. ¹H NMR (CDCl₃): 10.08 (s, 1H), 9.89 (br s, 1H), 8.44 (dd, J=7.5, 1.0, 1H), 7.90 (s, 1H), 7.36-7.28 (m, 2H), 3.78 (br s, 4H), 3.54 (br s, 4H), 1.50 (s, 9H).

Step B: 4-(3-Morpholin-4-ylmethyl-1H-indole-7-carbonyl)-piperazine-1-carboxylic acid tert-butyl ester. To a solution of 4-(3-formyl-1H-indole-7-carbonyl)-piperazine-1-carboxylic acid tert-butyl ester (0.32 g, 0.89 mmol) and morpholine (86 mg, 0.99 mmol) in DCM (9 mL) was added NaBH(OAc)₃ (475 mg, 2.24 mmol). After 24 h, the solution was concentrated and the resulting residue was partitioned between EtOAc and 1 N NaOH (50 mL). The organic layer was washed with brine (50 mL), dried, and concentrated to provide 300 mg (79%) of the title compound as an amber oil. MS (ESI): mass calcd. for C₂₃H₃₂N₄O₄, 428.54; m/z found, 429.2 [M+H]⁺. ¹H NMR (CDCl₃): 9.08 (s, 1H), 7.86 (d, J=8.0, 1H), 7.21-7.18 (m, 2H), 7.12 (t, J=7.5, 1H), 3.74-3.70 (m, 10H), 3.51 (br s, 4H), 2.49 (br s, 4H), 1.49 (s, 9H).

Step C: (3-Morpholin-4-ylmethyl-1H-indol-7-yl)-piperazin-1-yl-methanone. To a solution of 4-(3-morpholin-4-ylmethyl-1H-indole-7-carbonyl)-piperazine-1-carboxylic acid tert-butyl ester (180 mg, 0.42 mmol) in DCM (4 mL) was added TFA (1 mL). After 4 h, the solution was concentrated and the resulting residue was dissolved in MeOH (8 mL) and treated with DOWEX® basic resin. After 2 h, the suspension was filtered and concentrated to provide 130 mg (94%) of the title compound as an amber oil. MS (ESI): mass calcd. for C₁₈H₂₄N₄O₄, 328.42; m/z found, 329.2 [M+H]⁺. ¹H NMR (CDCl₃): 9.01 (s, 1H), 7.85 (d, J=8.0, 1H), 7.23-7.20 (m, 2H), 7.12 (t, J=7.5, 1H), 3.76-3.71 (m, 10H), 2.94 (br s, 4H), 2.50 (br s, 4H).

Step D: (4-Isopropyl-piperazin-1-yl)-(3-morpholin-4-ylmethyl-1H-indol-7-yl)-methanone. To a solution of (3-morpholin-4-ylmethyl-1H-indol-7-yl)-piperazin-1-yl-methanone (53 mg, 0.16 mmol) and acetone (9.4 mg, 0.16 mmol) in DCM (2 mL) was added NaBH(OAc)₃ (86 mg, 0.40 mmol). After 24 h, the solution was concentrated and the resulting residue was partitioned between EtOAc and 1 N NaOH (25 mL). The organic layer was washed with brine (25 mL), dried, and concentrated to provide 14 mg (24%) of the title compound as a colorless oil. LC/MS: Rₜ=1.47. MS (ESI): mass calcd. for C₂₁H₃₀N₄O₂, 370.50; m/z found, 371.2 [M+H]⁺. ¹H NMR (CDCl₃): 9.06 (br s, 1H), 7.84 (d, J=7.5, 1H), 7.28-7.19 (m, 2H), 7.11 (t, J=7.5, 1H), 3.79-3.70 (m, 10H), 2.94 (br s, 1H), 2.75 (h, J=6.5, 1H), 2.58 (br s, 3H), 2.50 (br s, 4H), 1.07 (d, J=6.5, 6H).

The compounds in Example 21-23 were prepared using methods analogous to those described for Example 20.

### Example 21

### (4-Cyclobutyl-piperazin-1-yl)-(3-morpholin-4-ylmethyl-1H-indol-7-yl)-methanone

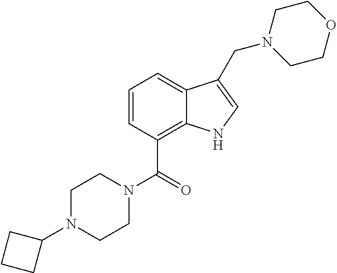

LC/MS: Rₜ=1.52. MS (ESI): mass calcd. for C₂₂H₃₀N₄O₂, 382.51; m/z found, 383.2 [M+H]⁺. ¹H NMR (CDCl₃): 9.02 (br s, 1H), 7.84 (d, J=8.0, 1H), 7.23-7.19 (m, 2H), 7.11 (t, J=7.5, 1H), 3.79 (br s, 4H), 3.72-3.70 (m, 6H), 2.77 (p, J=7.8, 1H), 2.50 (br s, 4H), 2.39 (br s, 4H), 2.11-2.03 (m, 2H), 1.94-1.78 (m, 2H), 1.76-1.68 (m, 2H).

### Example 22

### (4-Isopropyl-piperazin-1-yl)-(3-piperidin-1-ylmethyl-1H-indol-7-yl)-methanone

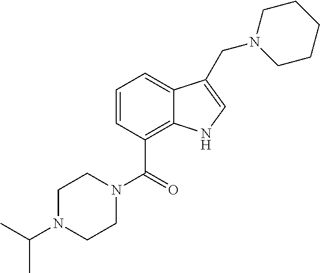

LC/MS: Rₜ=1.52. MS (ESI): mass calcd. for C₂₂H₃₂N₄O, 368.53; m/z found, 369.3 [M+H]⁺. ¹H NMR (CDCl₃): 9.50 (br s, 1H), 7.69 (d, J=7.9, 1H), 7.51 (d, J=2.0, 1H), 7.26-7.24 (m, 1H), 7.16 (t, J=7.9, 1H), 4.22 (s, 2H), 3.80 (br s, 4H), 2.92 (br s, 4H), 2.78 (h, J=6.5, 1H), 2.60 (br s, 4H), 1.84 (br s, 4H), 1.49 (br s, 2H), 1.07 (d, J=6.5, 6H).

### Example 23

### (4-Cyclobutyl-piperazin-1-yl)-(3-piperidin-1-ylmethyl-1H-indol-7-yl)-methanone

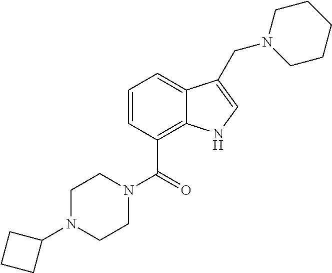

LC/MS: Rₜ=3.24. MS (ESI): mass calcd. for C₂₃H₃₂N₄O, 380.54; m/z found, 381.3 [M+H]⁺. ¹H NMR (CDCl₃): 9.02 (br s, 1H), 7.81 (d, J=8.0, 1H), 7.21-7.19 (m, 2H), 7.09 (t, J=7.5, 1H), 3.79 (br s, 4H), 3.71 (s, 2H), 2.77 (p, J=8.0, 1H), 2.45-2.38 (m, 8H), 2.09-2.03 (m, 2H), 1.94-1.87 (m, 2H), 1.78-1.68 (m, 2H), 1.60-1.56 (m, 4H), 1.42 (br s, 2H).

### Example 24

### (4-Isopropyl-piperazin-1-yl)-(3-piperidin-1-ylmethyl-1H-indol-6-yl)-methanone

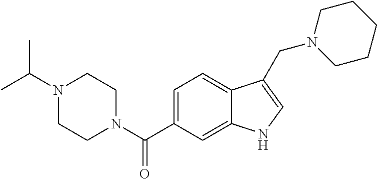

Step A: 3-Formyl-1H-indole-6-carboxylic acid. To a solution of methyl 3-formyl-indole-6-carboxylic acid (4.0 g, 19.7 mmol) in tetrahydrofuran (THF):H₂O (3:1; 100 mL) was added 2 N LiOH (20 mL, 39.4 mmol). After 24 h, the reaction mixture was partially concentrated and diluted with H₂O (100 mL). The solution was cooled to 0° C. and treated with conc. HCl until a precipitate formed. The solid was collected and dried under vacuum to provide 4.0 g (100%) of the title compound as a tan solid. ¹H NMR (DMSO-d₆): 12.85 (br s, 1H), 12.54 (br s, 1H), 9.98 (s, 1H), 8.49 (d, J=4.0, 1H), 8.17-8.14 (m, 2H), 7.83 (dd, J=8.5, 1.9, 1H).

Step B: 6-(4-Isopropyl-piperazine-1-carbonyl)-1H-indole-3-carbaldehyde. To a solution of formyl-1H-indole-6-carboxylic acid (4.0 g, 21.1 mmol), 1-isopropyl-piperazine (2.7 g, 21.1 mmol) and HOBt (4.3 g, 31.7 mmol) in DMF (100 mL) was added EDC (6.1 g, 31.7 mmol). After 24 h, the solution was partitioned between EtOAc and 1 N NaOH (250 mL). The organic layer was washed with brine (250 mL), dried, and concentrated. The resulting residue was purified by FCC to provide 2.7 g (43%) of the title compound as a tan solid. MS (ESI): mass calcd. for C₁₇H₂₁N₃O₂, 299.38; m/z found, 300.2 [M+H]⁺. ¹H NMR (CDCl₃): 10.99 (br s, 1H), 9.98 (s, 1H), 8.27 (d, J=8.2, 1H), 7.76 (br s, 1H), 7.40 (br s, 1H), 7.26 (s, 1H), 3.86 (br s, 2H), 3.50 (br s, 2H), 2.76 (h, J=6.5, 1H), 2.65 (br s, 2H), 2.47 (br s, 2H), 1.07 (d, J=6.5, 6H).

Step C: (4-Isopropyl-piperazin-1-yl)-(3-piperidin-1-ylmethyl-1H-indol-6-yl)-methanone. To a solution of 6-(4-isopropyl-piperazine-1-carbonyl)-1H-indole-3-carbaldehyde (150 mg, 0.50 mmol) and piperidine (43 mg, 0.50 mmol) in DCM (5 mL) was added NaBH(OAc)₃ (266 mg, 1.25 mmol). After 24 h, the solution was concentrated and the resulting residue was partitioned between EtOAc and 1 N NaOH (20 mL). The organic layer was washed with brine (25 mL), dried, and concentrated to provide 185 mg (100%) of the title compound as a white solid. LC/MS: Rₜ=1.55. MS (ESI): mass calcd. for C₂₂H₃₂N₄O, 368.53; m/z found, 369.3 [M+H]⁺. ¹H NMR (CDCl₃): 8.72 (br s, 1H), 7.71 (d, J=8.0, 1H), 7.51 (s, 1H), 7.26 (br s, 1H), 7.16 (d, J=8.0, 1H), 3.82-3.55 (m, 4H), 3.73 (s, 2H), 2.74 (h, J=6.5, 1H), 2.65-2.40 (m, 8H), 1.63-1.57 (m, 4H), 1.43 (br s, 2H), 1.07 (d, J=6.5, 6H).

### Example 25

### (4-Isopropyl-piperazin-1-yl)-(1-methanesulfonyl-3-piperidin-1-ylmethyl-1H-indol-6-yl)-methanone

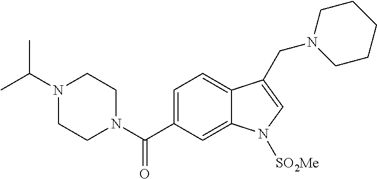

To a 0° C. solution of (4-isopropyl-piperazin-1-yl)-(3-piperidin-1-ylmethyl-1H-indol-6-yl)-methanone (100 mg, 0.27 mmol) in DMF (3 mL) was added NaH (16 mg, 0.41 mmol). The suspension was stirred for 15 min then warmed to rt over 15 min. The suspension was cooled to 0° C. and treated with methanesulfonyl chloride (47 mg, 0.41 mmol) and stirred at 0° C. for 2 h. The suspension was partitioned between EtOAc and brine (25 mL). The organic layer was washed with brine (25 mL), dried, and concentrated. The resulting residue was purified by FCC to provide 28 mg (23%) of the title compound as a white foam. LC/MS: Rₜ=3.18. MS (ESI): mass calcd. for C₂₃H₃₄N₄O₃S, 446.62; m/z found, 447.3 [M+H]⁺. ¹H NMR (CDCl₃): 7.96 (s, 1H), 7.83 (d, J=8.2, 1H), 7.41 (s, 1H), 7.35 (dd, J=8.2, 1.3, 1H), 3.83 (br s, 2H), 3.62 (s, 2H), 3.48 (br s, 2H), 3.12 (s, 3H), 2.74 (h, J=6.5, 1H), 2.64 (br s, 2H), 2.55-2.45 (m, 6H), 1.61-1.56 (m, 4H), 1.45 (br s, 2H), 1.06 (d, J=6.5, 6H).

### Example 26

### (4-Isopropyl-piperazin-1-yl)-(1-methanesulfonyl-3-morpholin-4-ylmethyl-1H-indol-6-yl)-methanone

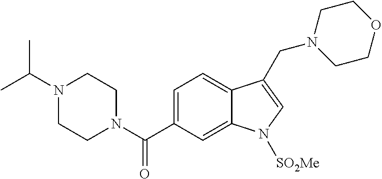

The title compound was prepared using methods analogous to those described for Example 25. LC/MS: Rₜ=1.53. MS (ESI): mass calcd. for C₂₂H₃₂N₄O₄S, 448.59; m/z found, 449.2 [M+H]⁺. ¹H NMR (CDCl₃): 7.96 (br s, 1H), 7.84 (d, J=8.5, 1H), 7.43 (s, 1H), 7.36 (dd, J=8.5, 1.2, 1H), 3.83 (br s, 2H), 3.73-3.71 (m, 4H), 3.65 (s, 2H), 3.48 (br s, 2H), 3.14 (s, 3H), 2.74 (h, J=6.5, 1H), 2.63 (br s, 2H), 2.49 (br s, 6H), 1.06 (d, J=6.5, 6H).

The compounds in Examples 27-31 were prepared using methods analogous to those described for Example 24.

### Example 27

### [3-(4-Isopropyl-piperazin-1-ylmethyl)-1H-indol-6-yl]-piperidin-1-yl-methanone

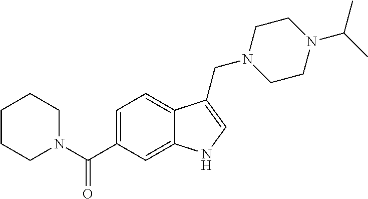

LC/MS: Rₜ=3.93. MS (ESI): mass calcd. for C₂₂H₃₂N₄O, 368.53; m/z found, 369.3 [M+H]⁺.

### Example 28

### [3-(4-Cyclopropyl-piperazin-1-ylmethyl)-1H-indol-6-yl]-piperidin-1-yl-methanone

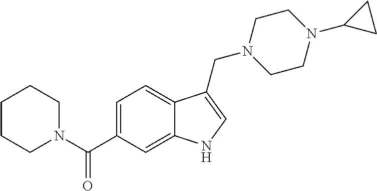

LC/MS: Rₜ=4.08. MS (ESI): mass calcd. for C₂₂H₃₀N₄O, 366.51; m/z found, 367.3 [M+H]⁺.

### Example 29

### [3-(4-Cyclobutyl-piperazin-1-ylmethyl)-1H-indol-6-yl]-piperidin-1-yl-methanone

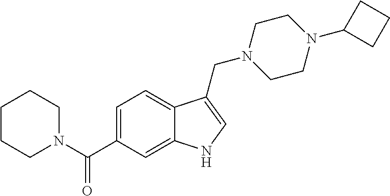

LC/MS: Rₜ=3.99. MS (ESI): mass calcd. for C₂₃H₃₂N₄O, 380.54; m/z found, 381.3 [M+H]⁺.

### Example 30

### [3-(4-Isopropyl-[1,4]diazepan-1-ylmethyl)-1H-indol-6-yl]-piperidin-1-yl-methanone

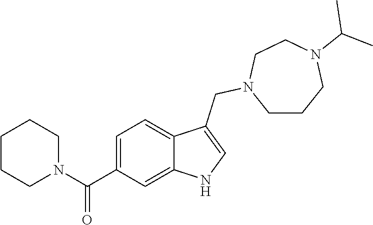

LC/MS: Rₜ=3.95. MS (ESI): mass calcd. for C₂₃H₃₄N₄O, 382.55; m/z found, 383.3 [M+H]⁺.

### Example 31

### [3-(4-Cyclopropyl-[1,4]diazepan-1-ylmethyl)-1H-indol-6-yl]-piperidin-1-yl-methanone

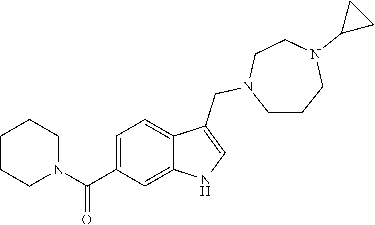

LC/MS: Rₜ=3.96. MS (ESI): mass calcd. for C₂₃H₃₂N₄O, 380.54; m/z found, 381.3 [M+H]⁺.

### Example 32

### (4-Isopropyl-piperazin-1-yl)-(3-piperidin-1-ylmethyl-1H-indol-5-yl)-methanone

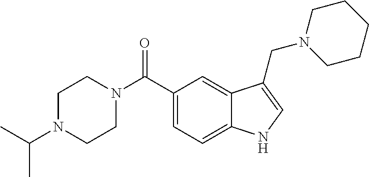

Step A: 3-Piperidin-1-ylmethyl-1H-indole-5-carboxylic acid methyl ester. To a solution of formaldehyde (37 wt % in H₂O; 240 mg, 0.6 mL, 8.0 mmol) in dioxane:acetic acid (4:1, 30 mL) was added piperidine (680 mg, 8.0 mmol). After 15 min, the solution was treated with 1H-indole-5-carboxylic acid methyl ester (1.0 mg, 5.7 mmol). After 3 h, the solution was concentrated and the resulting residue was partitioned between EtOAc and 1 N NaOH (50 mL). The organic layer was washed with brine (50 mL), dried, and concentrated. The resulting residue was purified by FCC to provide 0.63 g (41%) of the title compound as an amber oil. MS (ESI): mass calcd. for C₁₆H₂₀N₂O₂, 272.35; m/z found, 273.2 [M+H]⁺. ¹H NMR (CDCl₃): 9.11 (br s, 1H), 8.46 (d, J=0.6, 1H), 7.87 (dd, J=8.6, 1.6, 1H), 7.29 (d, J=8.6, 1H), 7.12 (d, J=2.1, 1H), 3.96 (s, 3H), 3.72 (s, 2H), 2.48 (br s, 4H), 1.60-1.55 (m, 4H), 1.43-1.40 (m, 2H).

Step B: Potassium 3-piperidin-1-ylmethyl-1H-indole-5-carboxylate. To a solution of 3-piperidin-1-ylmethyl-1H-indole-5-carboxylic acid methyl ester (0.60 g, 2.2 mmol) in i-PrOH (2 mL) was added 2 N KOH (1.2 mL, 2.4 mmol). The reaction mixture was heated at 55° C. for 24 h. The solution was concentrated to provide the title compound as a white solid in quantitative yield. The solid was used in the next step without further purification.

Step C: (4-Isopropyl-piperazin-1-yl)-(3-piperidin-1-ylmethyl-1H-indol-5-yl)-methanone. To a solution of potassium 3-piperidin-1-ylmethyl-1H-indole-5-carboxylate (200 mg, 0.67 mmol), 1-isopropyl-piperazine (95 mg, 0.74 mmol) and HOBt (135 mg, 1.0 mmol) in DMF (3.4 mL) was added EDC (192 mg, 1.0 mmol). After 24 h, the solution was partitioned between EtOAc and 1 N NaOH (25 mL). The organic layer was washed with brine (250 mL), dried, and concentrated. The resulting residue was purified by FCC to provide 36 mg (15%) of the title compound as a colorless oil. LC/MS: Rₜ=3.33. MS (ESI): mass calcd. for C₂₂H₃₂N₄O, 368.53; m/z found, 369.3 [M+H]⁺. ¹H NMR (CDCl₃): 8.88 (br s, 1H), 7.84 (s, 1H), 7.26-7.21 (m, 2H), 7.11 (br s, 1H), 3.84-3.56 (br s, 4H), 3.68 (s, 2H), 2.73 (h, J=6.4, 1H), 2.54-2.43 (m, 8H), 1.57-1.52 (m, 4H), 1.40 (br s, 2H), 1.06 (d, J=6.4, 6H).

The compounds in Examples 33-34 were prepared using methods analogous to those described for Example 32.

### Example 33

### (4-Cyclobutyl-piperazin-1-yl)-(3-piperidin-1-ylmethyl-1H-indol-5-yl)-methanone

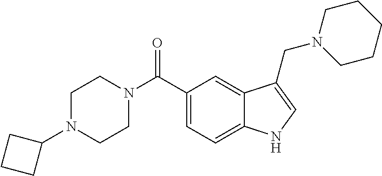

LC/MS: Rₜ=3.48. MS (ESI): mass calcd. for C₂₃H₃₂N₄O, 380.54; m/z found, 381.3 [M+H]⁺. ¹H NMR (CDCl₃): 9.29 (br s, 1H), 7.82 (s, 1H), 7.20 (br s, 2H), 7.08 (br s, 1H), 3.93-3.58 (m, 4H), 3.67 (s, 2H), 2.76 (h, J=7.8, 1H), 2.55-2.26 (m, 8H), 2.07-2.01 (m, 2H), 1.93-1.84 (m, 2H), 1.75-1.70 (m, 2H), 1.56-1.51 (m, 4H), 1.40-1.37 (m, 2H).

### Example 34

### (4-Isopropyl-[1,4]diazepan-1-yl)-(3-piperidin-1-ylmethyl-1H-indol-5-yl)-methanone

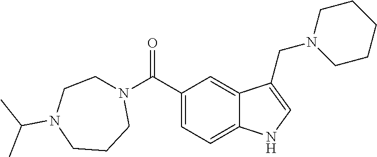

LC/MS: Rₜ=3.35. MS (ESI): mass calcd. for C₂₃H₃₄N₄O, 382.55; m/z found, 383.3 [M+H]⁺. ¹H NMR (CDCl₃): 8.88 (br s, 1H), 7.79 (s, 1H), 7.25-7.19 (m, 2H), 7.11 (br s, 1H), 3.80 (br s, 2H), 3.67 (s, 2H), 3.54-3.50 (m, 2H), 2.99-2.93 (m, 1H), 2.83 (br s, 1H), 2.73-2.68 (br s, 1H), 2.64-2.60 (br s, 2H), 2.42 (br s, 4H), 1.95 (br s, 1H), 1.75 (br s, 1H), 1.57-1.52 (m, 4H), 1.40-1.37 (m, 2H), 1.04 (d, J=6.0, 3H), 0.98 (d, J=6.0, 3H).

### Example 35

### (4-Isopropyl-piperazin-1-yl)-(3-morpholin-4-ylmethyl-1H-indol-5-yl)-methanone

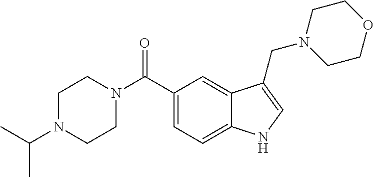

Step A: (1H-Indol-5-yl)-(4-isopropyl-piperazin-1-yl)-methanone. The title compound was prepared from 1H-Indole-5-carboxylic acid using methods analogous to those described in Example 32, Step C. MS (ESI): mass calcd. for C₁₆H₂₁N₃O, 271.37; m/z found, 272.2 [M+H]⁺. Human H₃ Kᵢ=97 nM.

Step B. The title compound was prepared using methods analogous to those described in Example 32, Step A. LC/MS: Rₜ=3.13. MS (ESI): mass calcd. for C₂₁H₃₀N₄O₂, 370.50; m/z found, 371.3 [M+H]⁺.

The compounds in Examples 36-42 were prepared using methods analogous to those described in Example 35.

### Example 36

### [3-(4-Cyclobutyl-piperazin-1-ylmethyl)-1H-indol-7-yl]-piperidin-1-yl-methanone

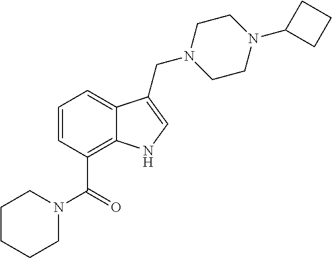

The title compound was prepared from (1H-indol-7-yl)-piperidin-1-yl-methanone. LC/MS: Rₜ=4.02. MS (ESI): mass calcd. for C₂₃H₃₂N₄O, 380.54; m/z found, 381.2 [M+H]⁺.

### Example 37

### [3-(4-Cyclobutyl-piperazin-1-ylmethyl)-1H-indol-4-yl]-piperidin-1-yl-methanone

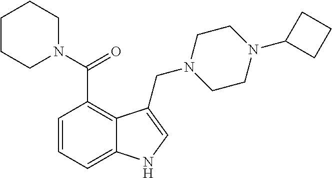

The title compound was prepared from (1H-indol-4-yl)-piperidin-1-yl-methanone. LC/MS: Rₜ=4.25. MS (ESI): mass calcd. for C₂₃H₃₂N₄O, 380.54; m/z found, 381.3 [M+H]⁺.

### Example 38

### [3-(4-Cyclobutyl-piperazin-1-ylmethyl)-1H-indol-5-yl]-piperidin-1-yl-methanone

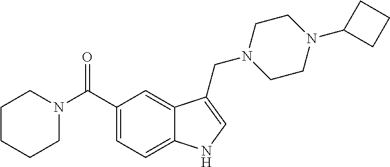

The title compound was prepared from (1H-indol-5-yl)-piperidin-1-yl-methanone. LC/MS: Rₜ=4.00. MS (ESI): mass calcd. for C₂₃H₃₂N₄O, 380.54; m/z found, 381.3 [M+H]⁺.

### Example 39

### (4-Isopropyl-piperazin-1-yl)-(3-morpholin-4-ylmethyl-1H-indol-4-yl)-methanone

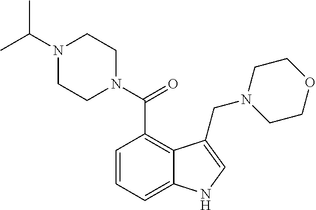

LC/MS: Rₜ=3.41. MS (ESI): mass calcd. for C₂₁H₃₀N₄O₂, 370.50; m/z found, 371.3 [M+H]⁺.

### Example 40

### (4-Cyclobutyl-piperazin-1-yl)-(3-morpholin-4-ylmethyl-1H-indol-5-yl)-methanone

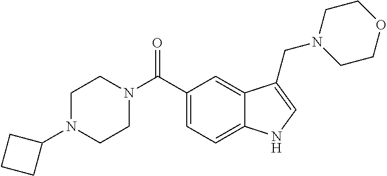

LC/MS: Rₜ=3.25. MS (ESI): mass calcd. for C₂₂H₃₀N₄O₂, 382.51; m/z found, 383.2 [M+H]⁺.

### Example 41

### (4-Cyclobutyl-piperazin-1-yl)-(3-morpholin-4-ylmethyl-1H-indol-4-yl)-methanone

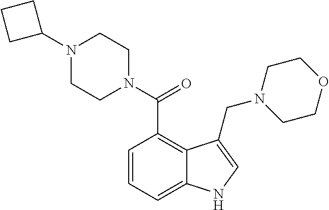

LC/MS: Rₜ=3.54. MS (ESI): mass calcd. for C₂₂H₃₀N₄O₂, 382.51; m/z found, 383.3 [M+H]⁺.

### Example 42

### (4-Cyclobutyl-piperazin-1-yl)-(3-piperidin-1-ylmethyl-1H-indol-4-yl)-methanone

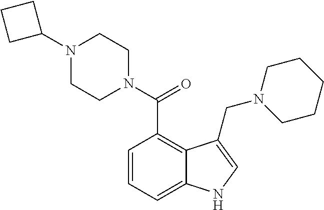

LC/MS: Rₜ=3.81. MS (ESI): mass calcd. for C₂₂H₃₂N₄O, 380.54; m/z found, 381.3 [M+H]⁺.

### Example 43

### [3-(4-Cyclobutyl-piperazine-1-ylmethyl)-1H-indol-6-yl]-(4-phenyl-piperidin-1-yl)-methanone

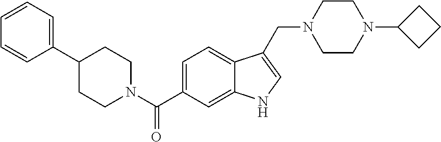

Step A: 6-(4-Phenyl-piperidine-1-carbonyl)-1H-indole-3-carbaldehyde. To a solution of 3-formyl-1H-indole-6-carboxylic acid (1.5 g, 7.9 mmol), 4-phenyl piperidine (1.53 g, 9.5 mmol), 1-hydroxy-7-azabenzotriazole (HOAT; 0.5 M in DMF, 32 mL, 15.8 mmol), and triethylamine (TEA; 3.29 mL, 23.7 mmol) in DMF (47 mL) was added bromotripyrrolidinophosphonium hexafluorophosphate (PyBrop; 7.36 g, 15.8 mmol). After 24 h, the reaction mixture was diluted with EtOAc (200 mL), washed with H₂O (100 mL×3) and brine (100 mL), and the aqueous layer was extracted with DCM (100 mL×2). The combined organic layers were dried and concentrated. The resulting residue was purified by FCC (100% EtOAc) to provide 1.2 g (46%) of the title compound. MS (ESI): mass calcd. for C₂₁H₂₀N₂O₂, 332.4; m/z found, 333.2 [M+H]⁺. ¹H NMR (DMSO-d₆): 12.26 (5, 1H), 9.97 (5, 1H), 8.39 (5, 1H), 8.13 (d, J=8.5, 1H), 7.59 (5, 1H), 7.33-7.28 (m, 5H), 7.21 (t, J=6.5, 1H), 3.11-2.92 (m, 4H), 2.82 (tt, J=12.0, 3.5, 1H), 1.79 (m, 2H), 1.68-1.60 (m, 2H).

Step B: [3-(4-Cyclopropyl-piperazine-1-ylmethyl)-1H-indol-6-yl]-(4-phenyl-piperidin-1-yl)-methanone. To a solution of 6-(4-phenyl-piperidine-1-carbonyl)-1H-indole-3-carbaldehyde (62 mg, 0.19 mmol) and 1-isopropyl-piperazine (31 mg, 0.23 mmol) in DMF (2.0 mL) was added macroporous reticular triacetoxy borohydride resin (350 mg, 2.17 mmol). The reaction mixture was placed on a shaker plate for 24 h. The reaction mixture was filtered and the resin washed with DMF (2×10 mL). The reaction mixture was concentrated and the resulting residue was purified by reverse phase HPLC yielding 31 mg (37%) of the title compound. LC/MS: Rₜ=10.54. MS (ESI): mass calcd. for C₂₉H₃₆N₄O, 456.6; m/z found, 457.2 [M+H]⁺. ¹H NMR (CDCl₃): 8.48 (5, 1H), 7.75 (d, J=8.5, 1H), 7.53 (5, 1H), 7.35 (t, J=7.5, 2H), 7.26-7.19 (m, 5H), 3.75 (5, 2H), 3.19-2.86 (m, 2H), 2.84-2.78 (m, 1H), 2.77-2.71 (m, 1H), 2.67-2.10 (m, 7H), 2.05-1.99 (m, 3H), 1.91-1.83 (m, 3H), 1.76-1.66 (m, 7H).

The compounds in Examples 44-101 were prepared using methods analogous to those described in the preceding examples.

### Example 44

### [3-(3-Hydroxymethyl-piperidin-1-ylmethyl)-1H-indol-6-yl]-(4-isopropyl-piperazin-1-yl)-methanone

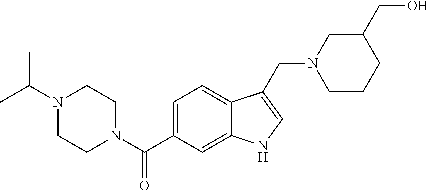

LC/MS: Rₜ=7.25. MS (ESI): mass calcd. for C₂₃H₃₄N₄O₂, 398.3; m/z found, 399.4 [M+H]⁺. ¹H NMR (CDCl₃): 8.77 (s, 1H), 7.70 (d, J=8.5, 1H), 7.50 (s, 1H), 7.19-7.15 (m, 2H), 3.81-3.85 (m, 4H), 2.82-2.50 (m, 8H), 2.31 (m, 1H), 2.20 (m, 1H), 1.81-1.51 (m, 8H), 1.23 (m, 1H), 1.07 (d, J=6.5, 6H).

### Example 45

### (4-Isopropyl-piperazin-1-yl)-[3-(4-phenyl-piperazin-1-ylmethyl)-1H-indol-6-yl]-methanone

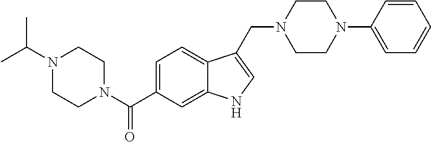

LC/MS: Rₜ=8.68. MS (ESI): mass calcd. for C₂₇H₃₅N₅O, 445.3; m/z found, 446.5 [M+H]⁺. ¹H NMR (CDCl₃): 8.41 (5, 1H), 7.79 (d, J=8.0, 1H), 7.52 (5, 1H), 7.27-7.25 (m, 3H), 7.18 (dd, J=8.5, 1.5, 1H), 6.94 (d, J=7.7, 2H), 6.86 (t, J=7.0, 1H), 3.84-3.72 (m, 2H), 3.79 (5, 2H), 3.66-3.51 (m, 2H), 3.22 (t, J=4.5, 4H), 2.77-2.72 (m, 1H), 2.68 (t, J=5.0, 4H), 2.65-2.46 (m, 4H), 1.08 (d, J=6.5, 6H).

### Example 46

### (4-Isopropyl-piperazin-1-yl)-[3-(4-pyridin-2-yl-piperazin-1-ylmethyl)-1H-indol-6-yl]-methanone

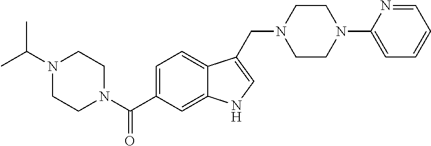

LC/MS: Rₜ=7.91. MS (ESI): mass calcd. for C₂₆H₃₄N₆O, 446.3; m/z found, 447.4 [M+H]⁺. ¹H NMR (CDCl₃): 8.42 (5, 1H), 8.19 (d, J=3.5, 1H), 7.79 (d, J=8.5, 1H), 7.52 (s, 1H), 7.49-7.46 (m, 1H), 7.25 (d, J=2.5, 1H), 7.18 (dd, J=8.0, 1.5, 1H), 6.65-6.61 (m, 2H), 3.87-3.76 (m, 2H), 3.78 (2H), 3.65-3.51 (m, 2H), 3.56 (t, J=5.0, 4H), 2.78-2.72 (m, 1H), 2.67-2.46 (m, 4H), 2.62 (t, J=5.0, 4H), 1.08 (d, J=6.5, 6H).

### Example 47

### (4-Isopropyl-piperazin-1-yl)-(3-thiomorpholin-4-ylmethyl-1H-indol-6-yl)-methanone

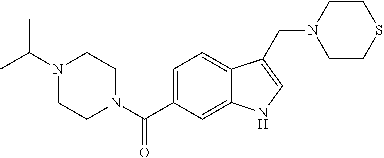

LC/MS: Rₜ=7.83. MS (ESI): mass calcd. for C₂₁H₃₀N₅O, 386.2; m/z found 387.4 [M+H]⁺.

### Example 48

### 1-{4-[6-(4-Isopropyl-piperazine-1-carbonyl)-1H-indol-3-ylmethyl]-piperazin-1-yl}-ethanone

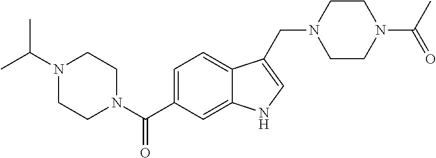

LC/MS: Rₜ=6.72. MS (ESI): mass calcd. for C₂₃H₃₃N₅O₂, 411.3; m/z found, 412.4 [M+H]⁺. ¹H NMR (CDCl₃): 8.70 (s, 1H), 7.72 (d, J=8.0, 1H), 7.51 (s, 1H), 7.19 (d, J=2.4, 1H), 7.15 (dd, J=8.0, 1.2, 1H), 3.87-3.75 (m, 2H), 3.72 (s, 2H), 3.61 (t, J=4.8, 2H), 3.58-3.49 (m, 2H), 3.44 (t, J=4.8, 2H), 2.76-2.64 (m, 1H), 2.64-2.44 (m, 6H), 2.07 (s, 3H), 1.05 (d, J=6.4, 6H).

### Example 49

### (4-Isopropyl-piperazin-1-yl)-[3-(4-thiazol-2-yl-piperazin-1-ylmethyl)-1H-indol-6-yl]-methanone

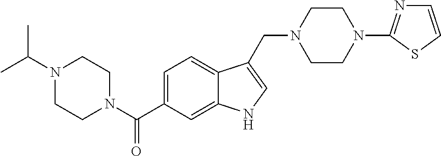

LC/MS: Rₜ=7.72. MS (ESI): mass calcd. for C₂₄H₃₂N₆OS, 452.6; m/z found, 453.4 [M+H]⁺.

### Example 50

### 1-{4-[6-(4-Isopropyl-piperazine-1-carbonyl)-1H-indol-3-ylmethyl]-[1,4]diazepam-1-yl}-ethanone

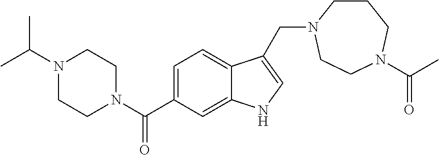

LC/MS: Rₜ=6.62. MS (ESI): mass calcd. for C₂₄H₃₅N₅O₂, 425.6; m/z found, 426.4 [M+H]⁺. ¹H NMR (CDCl₃): 8.32 (s, 1H), 7.76 (dd, J=13.0, 8.5, 1H), 7.51 (d, J=6.5, 1H), 7.21-7.16 (m, 2H), 3.91-3.71 (m, 2H), 3.84 (d, J=5.0, 2H), 3.68-3.49 (m, 2H), 3.67-3.63 (m, 2H), 3.54-3.51 (t, J=12.5, 1H), 3.48-3.46 (m, 1H), 2.77-2.64 (m, 5H), 2.64-2.44 (m, 4H), 2.11-2.08 (m, 3H), 1.90-1.85 (m, 2H), 1.08 (d, J=6.5, 6H).

### Example 51

### [3-(4-Benzyl-piperazin-1-ylmethyl)-1H-indol-6-yl]-(4-isopropyl-piperazin-1-yl)-methanone

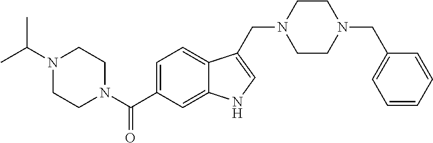

LC/MS: Rₜ=8.20. MS (ESI): mass calcd. for C₂₈H₃₇N₅O, 459.6; m/z found, 460.5 [M+H]⁺. ¹H NMR (CDCl₃): 8.46 (s, 1H), 7.73 (d, J=8.0, 1H), 7.49 (s, 1H), 7.31 (d, J=4.5, 4H), 7.27-7.23 (m, 1H), 7.20 (d, J=2.5, 1H), 7.16 (dd, J=8.5, 1.5, 1H), 3.88-3.76 (m, 2H), 3.74 (s, 2H), 3.62-3.50 (m, 2H), 3.52 (s, 2H), 2.77-2.66 (m, 1H), 2.66-2.41 (m, 12H), 1.07 (d, J=6.5, 6H).

### Example 52

### [3-(4-Biphenyl-4-yl-piperazin-1-ylmethyl)-1H-indol-6-yl]-(4-isopropyl-piperazin-1-yl)-methanone

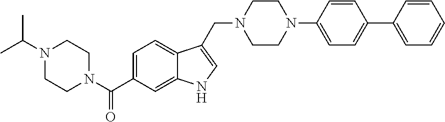

LC/MS: Rₜ=9.84. MS (ESI): mass calcd. for C₃₃H₃₉N₅O, 521.7; m/z found, 522.5 [M+H]⁺.

### Example 53

### [3-(4-Benzydryl-piperazin-1-ylmethyl)-1H-indol-6-yl]-(4-isopropyl-piperazin-1-yl)-methanone

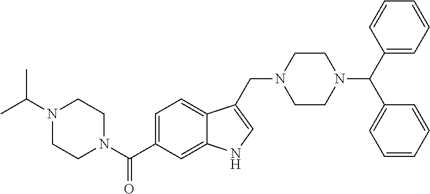

LC/MS: Rₜ=9.85. MS (ESI): mass calcd. for C₃₄H₄₁N₅O, 535.7; m/z found, 536.6 [M+H]⁺.

### Example 54

### (4-Isopropyl-piperazin-1-yl)-[3-(4-methanesulfonyl-piperazin-1-ylmethyl)-1H-indol-6-yl]-methanone

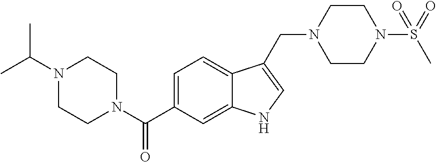

LC/MS: Rₜ=7.27. MS (ESI): mass calcd. for C₂₂H₃₃N₅O₃S, 447.6; m/z found, 448.4 [M+H]⁺. ¹H NMR (CDCl₃): 8.46 (s, 1H), 7.74 (d, J=8.0, 1H), 7.52 (s, 1H), 7.22 (d, J=2.5, 1H), 7.18 (dd, J=8.0, 1.5, 1H), 3.91-3.70 (m, 2H), 3.77 (s, 2H), 3.68-3.48 (m, 2H), 3.25 (t, J=4.5, 4H), 2.81-2.73 (m, 1H), 2.79 (s, 3H), 2.66-2.46 (m, 4H), 2.61 (t, J=4.5, 4H), 1.08 (d, J=7.0, 6H).

### Example 55

### [3-(4-Benzyl-piperidin-1-ylmethyl)-1H-indol-6-yl]-(4-isopropyl-piperazin-1-yl)-methanone

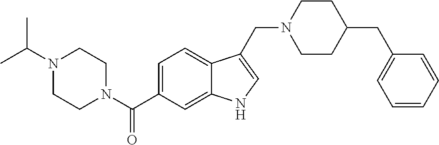

LC/MS: Rₜ=9.91. MS (ESI): mass calcd. for C₂₉H₃₈N₄O, 458.6; m/z found, 459.5 [M+H]⁺. ¹H NMR (CDCl₃): 8.52 (s, 1H), 7.70 (d, J=8.0, 1H), 7.50 (s, 1H), 7.29-7.26 (m, 2H), 7.22-7.13 (m, 5H), 3.88-3.76 (m, 2H), 3.73 (s, 2H), 3.65-3.48 (m, 2H), 2.98 (d, J=11.0, 2H), 2.77-2.72 (m, 1H), 2.66-2.43 (m, 4H), 2.54 (d, J=7.0, 2H), 1.99 (t, J=11.0, 2H), 1.63 (d, J=14.0, 2H), 1.55-1.48 (m, 1H), 1.38-1.28 (m, 2H), 1.07 (d, J=6.5, 6H).

### Example 56

### (4-Isopropyl-piperazin-1-yl)-[3-(4-phenethyl-piperazin-1-ylmethyl)-1H-indol-6-yl]-methanone

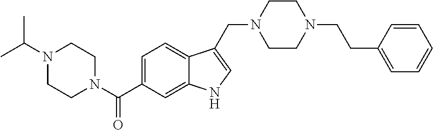

LC/MS: Rₜ=8.24. MS (ESI): mass calcd. for C₂₉H₃₉N₅O, 473.6; m/z found, 474.5 [M+H]⁺. ¹H NMR (CDCl₃): 8.42 (s, 1H), 7.75 (d, J=8.5, 1H), 7.51 (s, 1H), 7.31-7.28 (m, 2H), 7.25-7.17 (m, 5H), 3.90-3.72 (m, 2H), 3.77 (s, 2H), 3.67-3.47 (m, 2H), 2.83-2.43 (m, 17H), 1.08 (d, J=6.5, 6H).

### Example 57

### (4-Isopropyl-piperazin-1-yl)-[3-(4-phenyl-piperidin-1-ylmethyl)-1H-indol-6-yl]-methanone

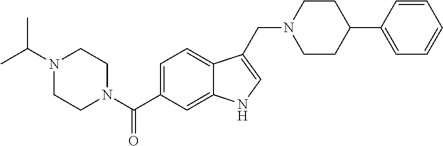

LC/MS: Rₜ=9.37. MS (ESI): mass calcd. for C₂₈H₃₅N₄O, 444.6; m/z found, 445.4 [M+H]⁺. ¹H NMR (CDCl₃): 8.45 (s, 1H), 7.77 (d, J=8.0, 1H), 7.52 (s, 1H), 7.32-7.28 (m, 2H), 7.24-7.19 (m, 4H), 3.89-3.75 (m, 2H), 3.81 (s, 2H), 3.68-3.50 (m, 2H), 3.14 (d, J=11.0, 2H), 2.77-2.71 (m, 1H), 2.64-2.47 (m, 5H), 2.20-2.15 (m, 2H), 1.86-1.84 (m, 4H), 1.08 (d, J=6.5, 6H).

### Example 58

### (4-Isopropyl-piperazin-1-yl)-(3-pyrrolidin-1-ylmethyl-1H-indol-6-yl)-methanone

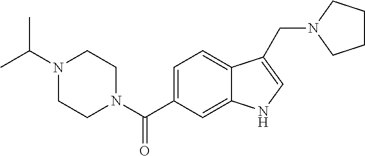

LC/MS: Rₜ=6.01. MS (ESI): mass calcd. for O₂₁H₃₀N₄O, 354.5; m/z found, 355.6 [M+H]⁺. ¹H NMR (CDCl₃): 8.67 (s, 1H), 7.71 (d, J=8.0, 1H), 7.50 (s, 1H), 7.22 (d, J=2.0, 1H), 7.16 (dd, J=8.0, 1.0, 1H), 3.91-3.71 (m, 2H), 3.83 (s, 2H), 3.68-3.47 (m, 2H), 2.77-2.71 (m, 1H), 2.66-2.42 (m, 4H), 2.60-2.57 (m, 4H), 1.81-1.78 (m, 4H), 1.07 (d, J=6.5, 6H).

### Example 59

### (4-Isopropyl-piperazin-1-yl)-(3-[1,4]-oxazepan-4-ylmethyl-1H-indol-6-yl)-methanone

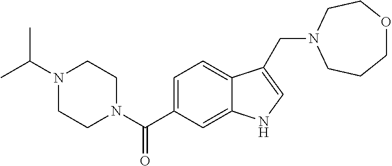

LC/MS: Rₜ=4.95. MS (ESI): mass calcd. for C₂₂H₃₂N₄O₂, 384.5; m/z found, 385.3 [M+H]⁺. ¹H NMR (CDCl₃): 8.73 (s, 1H), 7.77 (d, J=8.0, 1H), 7.52 (s, 1H), 7.20 (d, J=2.0, 1H), 7.16 (dd, J=8.0, 1.5, 1H), 3.93-3.69 (m, 2H), 3.85-3.82 (m, 4H), 3.73-3.71 (m, 2H), 3.66-3.44 (m, 2H), 2.77-2.72 (m, 5H), 2.66-2.43 (m, 4H), 1.94-1.89 (m, 2H), 1.07 (d, J=6.5, 6H).

### Example 60

### N-{1-[6-(4-Isopropyl-piperazine-1-carbonyl)-1H-indol-3-ylmethyl]-pyrrolidin-3-yl}-N-methyl-acetamide

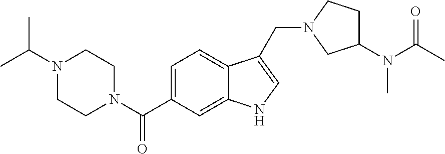

LC/MS: Rₜ=4.72. MS (ESI): mass calcd. for C₂₄H₃₅N₅O₂, 425.6; m/z found, 426.3 [M+H]⁺.

### Example 61

### (4-Isopropyl-piperazin-1-yl)-{3-[4-(morpholine-4-carbonyl)-piperazin-1-ylmethyl]-1H-indol-6-yl}-methanone

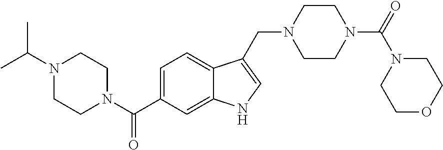

LC/MS: Rₜ=4.65. MS (ESI): mass calcd. for C₂₆H₃₈N₆O₃, 482.6; m/z found, 483.3 [M+H]⁺.

### Example 62

### (4-Isopropyl-piperazin-1-yl)-{3-[4-(pyridine-4-carbonyl)-piperazin-1-ylmethyl]-1H-indol-6-yl}-methanone

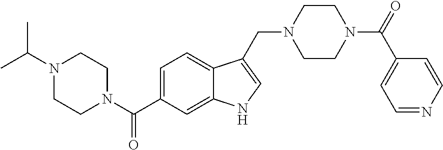

LC/MS: Rₜ=4.57. MS (ESI): mass calcd. for C₂₇H₃₄N₈O₂, 474.6; m/z found, 475.3 [M+H]⁺.

### Example 63

### [3-(4-Benzoyl-piperazin-1-ylmethyl)-1H-indol-6-yl]-(4-isopropyl-piperazin-1-yl)-methanone

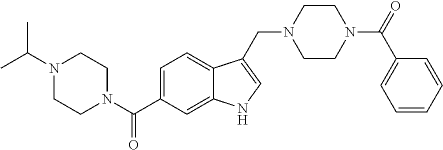

LC/MS: Rₜ=6.53. MS (ESI): mass calcd. for C₂₈H₃₅N₅O₂, 473.6; m/z found, 474.4 [M+H]⁺. ¹H NMR (CDCl₃): 8.34 (s, 1H), 7.94 (d, J=8.5, 2H), 7.77 (d, J=8.0, 1H), 7.56 (t, J=7.5, 1H), 7.52 (s, 1H), 7.47 (t, J=8.0, 2H), 7.24 (d, J=2.5, 1H), 7.18 (dd, J=8.0, 1.0, 1H), 3.88-3.69 (m, 2H), 3.78 (s, 2H), 3.68-3.52 (m, 2H), 3.28-3.21 (m, 1H), 3.07 (d, J=12.0, 2H), 2.77-2.72 (m, 1H), 2.69-2.45 (m, 4H), 2.21-2.16 (m, 2H), 1.89-1.85 (m, 4H), 1.08 (d, J=6.5, 6H).

### Example 64

### (4-Isopropyl-piperazin-1-yl)-[3-(4-pyridin-4-yl-piperazin-1-ylmethyl)-1H-indol-6-yl]-methanone

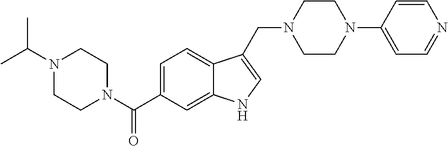

LC/MS: Rₜ=5.15. MS (ESI): mass calcd. for C₂₆H₃₄N₆O, 473.6; m/z found, 474.4 [M+H]⁺.

### Example 65

### [3-(4-Hydroxy-4-phenyl-piperidin-1-ylmethyl)-1H-indol-6-yl]-(4-isopropyl-piperazin-1-yl)-methanone

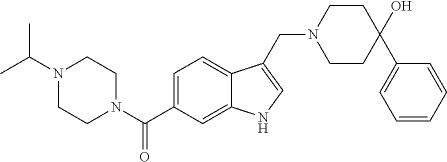

LC/MS: Rₜ=6.20. MS (ESI): mass calcd. for C₂₈H₃₆N₄O₂, 460.6; m/z found, 461.3 [M+H]⁺.

### Example 66

### (4-Isopropyl-piperazin-1-yl)-(3-thiomorpholin-4-ylmethyl-1H-indol-6-yl)-methanone

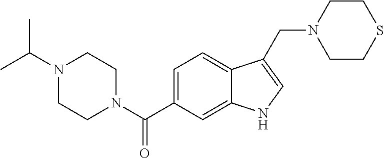

LC/MS: Rₜ=6.47. MS (ESI): mass calcd. for C₂₅H₃₀N₄OS, 434.6; m/z found, 435.2 [M+H]⁺.

### Example 67

### (3-[1,4′]Bipiperidinyl-1′-ylmethyl-1H-indol-6-yl)-thiomorpholin-4-yl-methanone

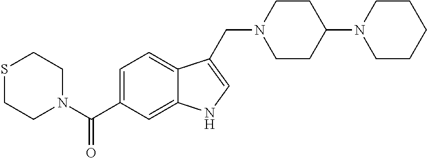

LC/MS: Rₜ=10.54. MS (ESI): mass calcd. for C₂₄H₃₄N₄OS, 426.6; m/z found, 427.2 [M+H]⁺.

### Example 68

### [3-(4-Cyclopentyl-piperazin-1-ylmethyl)-1H-indol-6-yl]-thiomorpholin-4-yl-methanone

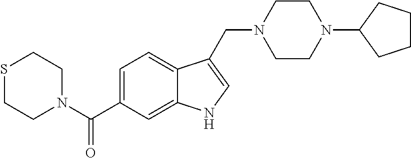

LC/MS: Rₜ=8.82. MS (ESI): mass calcd. for C₂₃H₃₂N₄OS, 412.6; m/z found, 413.1 [M+H]⁺.

### Example 69

### [3-(3-Dimethylamino-pyrrolidin-1-ylmethyl)-1H-indol-6-yl]-thiomorpholin-4-yl-methanone

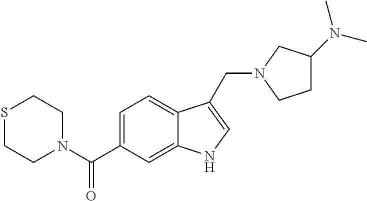

LC/MS: Rₜ=8.21. MS (ESI): mass calcd. for C₂₀H₂₈N₄OS, 372.5; m/z found, 373.1 [M+H]⁺.

### Example 70

### [3-(4-Cyclobutyl-piperazin-1-ylmethyl)-1H-indol-6-yl]-thiomorpholin-4-yl-methanone

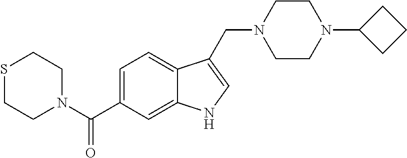

LC/MS: Rₜ=8.55. MS (ESI): mass calcd. for C₂₂H₃₀N₄OS, 398.5; m/z found, 399.1 [M+H]⁺.

### Example 71

### [3-(4-Cyclobutyl-[1,4]diazepan-1-ylmethyl)-1H-indol-6-yl]-thiomorpholin-4-yl-methanone

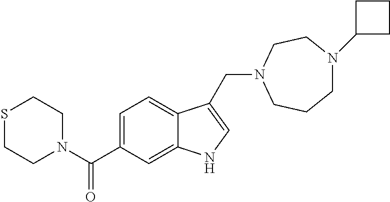

LC/MS: Rₜ=10.36. MS (ESI): mass calcd. for C₂₃H₃₂N₄OS, 412.6; m/z found, 413.1 [M+H]⁺. ¹H NMR (CDCl₃): 8.49 (s, 1H), 7.78 (d, J=8.5, 1H), 7.46 (s, 1H), 7.21 (d, J=2.5, 1H), 7.12 (dd, J=8.0, 1.5, 1H), 4.16-3.72 (m, 4H), 3.83 (s, 2H), 2.97-2.89 (m, 1H), 2.80-2.60 (m, 4H), 2.78 (t, J=6.0, 2H), 2.74-2.72 (m, 2H), 2.58 (t, J=6.0, 2H), 2.53-2.51 (m, 2H), 2.05-1.99 (m, 2H), 1.88-1.72 (m, 4H), 1.70-1.57 (m, 2H).

### Example 72

### [3-(4-Isopropyl-[1,4]diazepan-1-ylmethyl)-1H-indol-6-yl]-thiomorpholin-4-yl-methanone

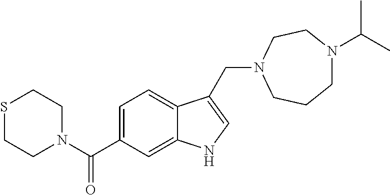

LC/MS: Rₜ=10.05. MS (ESI): mass calcd. for C₂₂H₃₂N₄OS, 400.6; m/z found, 401.1 [M+H]⁺. ¹H NMR (CDCl₃): 8.57 (s, 1H), 7.75 (d, J=8.0, 1H), 7.45 (s, 1H), 7.19 (d, J=2.4, 1H), 7.10 (dd, J=8.0, 1.6, 1H), 4.08-3.66 (m, 4H), 3.82 (s, 2H), 2.91-2.83 (m, 1H), 2.75-2.63 (m, 12H), 1.82-1.71 (m, 2H), 1.00 (d, J=6.8, 6H).

### Example 73

### (3-[1,4′]Bipiperidinyl-1′-ylmethyl-1H-indol-6-yl)-morpholin-4-yl-methanone

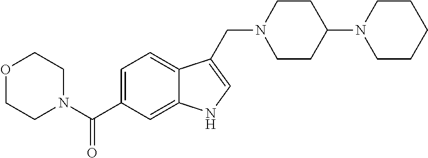

LC/MS: Rₜ=9.21. MS (ESI): mass calcd. for C₂₄H₃₄N₄O₂, 410.6; m/z found, 411.1 [M+H]⁺.

### Example 74

### [3-(4-Cyclopentyl-piperazin-1-ylmethyl)-1H-indol-6-yl]-morpholin-4-yl-methanone

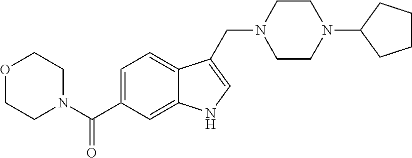

LC/MS: Rₜ=7.66. MS (ESI): mass calcd. for C₂₃H₃₂N₄O₂, 396.5; m/z found, 397.1 [M+H]⁺. ¹H NMR (CDCl₃): 8.47 (s, 1H), 7.75 (d, J=8.5, 1H), 7.50 (s, 1H), 7.23 (d, J=2.5, 1H), 7.16 (dd, J=8.0, 1.0, 1H), 3.78-3.60 (m, 8H), 3.74 (s, 2H), 2.80-2.30 (m, 8H), 1.89-1.81 (m, 2H), 1.73-1.64 (m, 3H), 1.58-1.50 (m, 2H), 1.43-1.35 (m, 2H).

### Example 75

### [3-(4-Cyclobutyl-piperazin-1-ylmethyl)-1H-indol-6-yl]-morpholin-4-yl-methanone

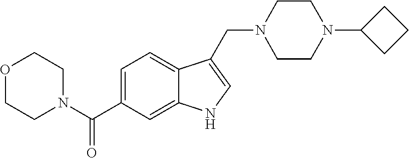

LC/MS: Rₜ=7.41. MS (ESI): mass calcd. for C₂₂H₃₀N₄O₂, 382.5; m/z found, 383.1 [M+H]⁺.

### Example 76

### [3-(4-Isopropyl-piperazin-1-ylmethyl)-1H-indol-6-yl]-morpholin-4-yl-methanone

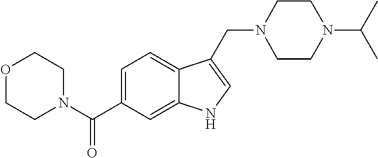

LC/MS: Rₜ=6.95. MS (ESI): mass calcd. for O₂₁H₃₀N₄O₂, 370.5; m/z found, 371.1 [M+H]⁺. ¹H NMR (CDCl₃): 8.54 (s, 1H), 7.74 (t, J=8.0, 1H), 7.51 (d, J=8.5, 1H), 7.23 (d, J=2.5, 1H), 7.16 (dd, J=8.0, 1.5, 1H), 3.78-3.61 (m, 8H), 3.74 (s, 2H), 2.69-2.46 (m, 6H), 2.68-2.61 (m, 1H), 2.29 (m, 2H), 1.05 (d, J=6.5, 6H).

### Example 77

### [3-(4-Cyclobutyl-[1,4]diazepan-1-ylmethyl)-1H-indol-6-yl]-morpholin-4-yl-methanone

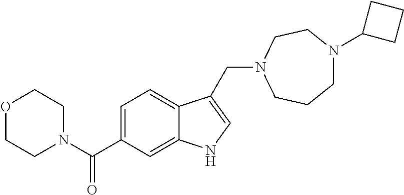

LC/MS: Rₜ=8.83. MS (ESI): mass calcd. for C₂₃H₃₂N₄O₂, 396.5; m/z found, 397.1 [M+H]⁺.

### Example 78

### [3-(4-Isopropyl-[1,4]diazepan-1-ylmethyl)-1H-indol-6-yl]-morpholin-4-yl-methanone

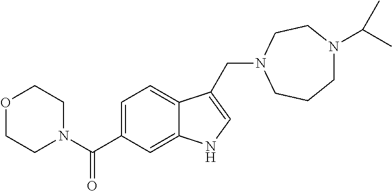

LC/MS: Rₜ=8.48. MS (ESI): mass calcd. for C₂₂H₃₂N₄O₂, 384.5; m/z found, 385.1 [M+H]⁺.

### Example 79

### (3-[1,4′]Bipiperidinyl-1′-ylmethyl-1H-indol-6-yl)-(4-hydroxymethyl-piperidin-1-yl)-methanone

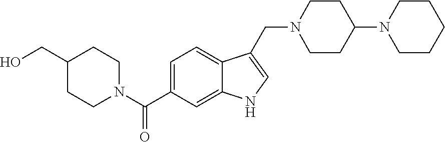

LC/MS: Rₜ=8.79. MS (ESI): mass calcd. for C₂₆H₃₈N₄O₂, 438.6; m/z found, 439.2 [M+H]⁺. ¹H NMR (CDCl₃): 8.44 (s, 1H), 7.74 (d, J=8.0, 1H), 7.47 (s, 1H), 7.22 (d, J=2.5, 1H), 7.15 (dd, J=8.5, 1.5, 1H), 3.71 (s, 2H), 3.56 (d, J=6.0, 2H), 3.04 (d, J=11.5, 2H), 3.01-2.79 (m, 2H), 2.54-2.47 (m, 5H), 2.27-2.20 (m, 1H), 2.01 (t, J=10.0, 2H), 1.86-1.74 (m, 5H), 1.68-1.55 (m, 7H), 1.49-1.39 (m, 3H), 1.35-1.21 (m, 2H),

### Example 80

### [3-(4-Cyclopentyl-piperazin-1-ylmethy)l-1H-indol-6-yl]-(4-hydroxymethyl-piperidin-1-yl)-methanone

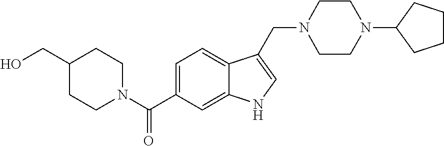

LC/MS: Rₜ=7.35. MS (ESI): mass calcd. for C₂₅H₃₆N₄O₂, 424.6; m/z found, 425.2 [M+H]⁺.

### Example 81

### [3-(4-Cyclobutyl-piperazin-1-ylmethy)l-1H-indol-6-yl]-(4-hydroxymethyl-piperidin-1-yl)-methanone

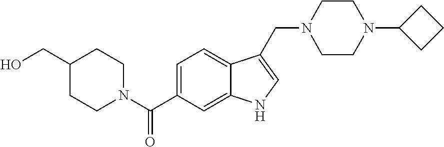

LC/MS: Rₜ=7.15. MS (ESI): mass calcd. for C₂₄H₃₄N₄O₂, 410.5; m/z found, 411.2 [M+H]⁺.

### Example 82

### (4-Hydroxymethyl-piperidin-1-yl)-[3-(4-isopropyl-piperazin-1-ylmethyl)-1H-indol-6-yl]-methanone

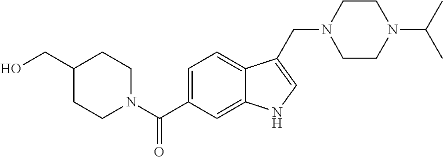

LC/MS: Rₜ=6.69. MS (ESI): mass calcd. for C₂₃H₃₄N₄O₂, 398.5; m/z found, 399.5 [M+H]⁺. ¹H NMR (CDCl₃): 8.67 (s, 1H), 7.73 (d, J=8.0, 1H), 7.46 (s, 1H), 7.22 (d, J=2.0, 1H), 7.15 (dd, J=8.5, 1.5, 1H), 3.75 (s, 2H), 3.53 (d, J=6.0, 2H), 2.67-2.62 (m, 1H), 2.62-2.49 (m, 6H), 1.86-1.76 (m, 3H), 1.75-1.66 (m, 7H), 1.36-1.16 (m, 2H), 1.05 (d, J=6.5, 6H).

### Example 83

### [3-(4-Cyclobutyl-[1,4]diazepan-1-ylmethyl)-1H-indol-6-yl]-(4-hydroxymethyl-piperidin-1-yl)-methanone

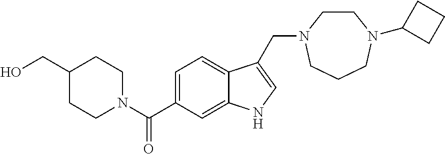

LC/MS: Rₜ=8.45. MS (ESI): mass calcd. for C₂₅H₃₆N₄O₂, 424.6; m/z found, 425.1 [M+H]⁺. ¹H NMR (CDCl₃): 8.62 (s, 1H), 7.75 (d, J=8.0, 1H), 7.47 (s, 1H), 7.20 (d, J=2.5, 1H), 7.15 (dd, J=8.5, 2.5, 1H), 3.83 (s, 2H), 3.54 (d, J=6.0, 2H), 3.07-2.81 (m, 3H), 2.98-2.89 (m, 2H), 2.78 (t, J=6.0, 2H), 2.73 (m, 2H), 2.57 (t, J=5.5, 2H), 2.53-2.51 (m, 2H), 2.04-1.99 (m, 2H), 1.88-1.78 (m, 6H), 1.70-1.57 (m, 4H), 1.37-1.17 (m, 2H).

### Example 84

### (4-Hydroxymethyl-piperidin-1-yl)-[3-(4-isopropyl-[1,4]diazepan-1-ylmethyl)-1H-indol-6-yl]-methanone

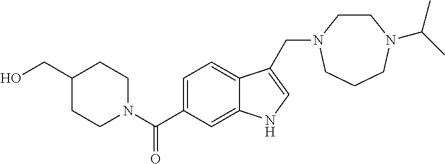

LC/MS: Rₜ=7.89. MS (ESI): mass calcd. for C₂₄H₃₆N₄O₂, 412.6; m/z found, 413.2 [M+H]⁺.

### Example 85

### (3-[1,4′]Bipiperidinyl-1′-ylmethyl-1H-indol-6-yl)-(4-phenyl-piperidin-1-yl)-methanone

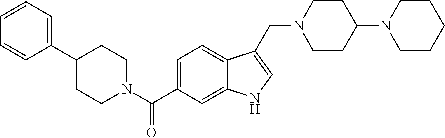

LC/MS: Rₜ=12.81. MS (ESI): mass calcd. for C₃₁H₄₀N₄O, 484.7; m/z found, 485.2 [M+H]⁺.

### Example 86

### [3-(4-Cyclopentyl-piperazin-ylmethyl)-1H-indol-6-yl]-(4-phenyl-piperidin-1-yl)-methanone

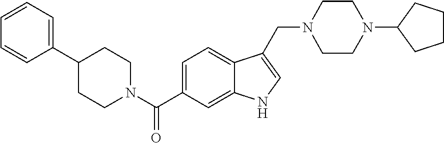

LC/MS: Rₜ=10.92. MS (ESI): mass calcd. for C₃₀H₃₈N₄O, 470.7; m/z found, 471.2 [M+H]⁺.

### Example 87

### [3-(3-Dimethylamino-pyrrolidin-1-ylmethyl)-1H-indol-6-yl]-(4-phenyl-piperidin-1-yl)-methanone

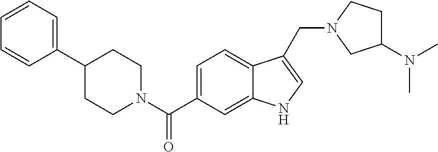

LC/MS: Rₜ=10.41. MS (ESI): mass calcd. for C₂₇H₃₄N₄O, 430.6; m/z found, 431.2 [M+H]⁺.

### Example 88

### [3-(4-Dimethylamino-piperidin-1-ylmethyl)-1H-indol-6-yl]-(4-phenyl-piperidin-1-yl)-methanone

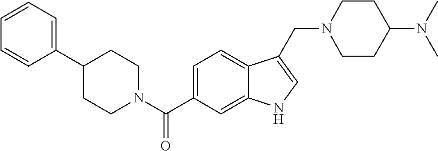

LC/MS: Rₜ=10.39. MS (ESI): mass calcd. for C₂₈H₃₆N₄O, 444.6; m/z found, 445.2 [M+H]⁺. ¹H NMR (CDCl₃): 8.49 (s, 1H), 7.75 (d, J=8.0, 1H), 7.54 (s, 1H), 7.35 (t, J=7.5, 2H), 7.26-7.23 (m, 4H), 7.20 (dd, J=8.0, 1.0, 1H), 3.72 (s, 2H), 3.20-2.85 (m, 2H), 3.03 (d, J=12.0, 2H), 2.84-2.78 (m, 1H), 2.28 (s, 6H), 2.16-2.10 (m, 1H), 2.02 (t, J=11.5, 2H), 1.98-1.84 (m, 2H), 1.80 (d, J=12.5, 2H), 1.81-1.64 (m, 4H), 1.59-1.51 (m, 2H).

### Example 89

### [3-(4-Cyclobutyl-[1,4]diazepan-1-ylmethyl)-1H-indol-6-yl]-(4-phenyl-piperidin-1-yl)-methanone

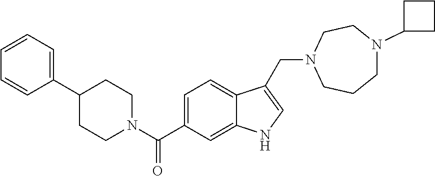

LC/MS: Rₜ=12.83. MS (ESI): mass calcd. for C₃₀H₃₈N₄O, 470.6; m/z found, 471.2 [M+H]⁺.

### Example 90

### Azepan-1-yl-(3-[1,4′]bipiperidinyl-1′-ylmethyl-1H-indol-6-yl)-methanone

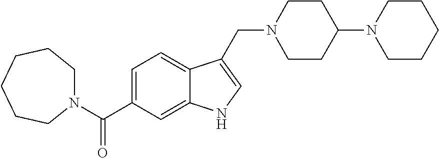

LC/MS: Rₜ=10.59. MS (ESI): mass calcd. for C₂₆H₃₈N₄O, 422.6; m/z found, 423.2 [M+H]⁺.

### Example 91

### Azepan-1-yl-[3-(4-cyclopentyl-piperazin-1-ylmethyl)-1H-indol-6-yl]-methanone

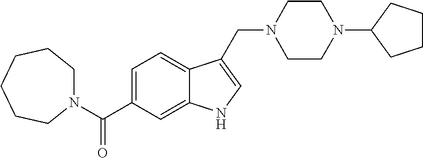

LC/MS: Rₜ=9.70. MS (ESI): mass calcd. for C₂₅H₃₆N₄O, 408.6; m/z found, 409.2 [M+H]⁺. ¹H NMR (CDCl₃): 8.43 (s, 1H), 7.73 (d, J=8.0, 1H), 7.43 (s, 1H), 7.20 (d, J=2.5, 1H), 7.14 (dd, J=8.0, 1.5, 1H), 3.74 (s, 2H), 3.73-3.69 (m, 2H), 3.49-3.44 (m, 2H), 2.74-2.38 (m, 8H), 1.91-1.82 (m, 4H), 1.72-1.59 (m, 9H), 1.56-1.50 (m, 2H), 1.43-1.36 (m, 2H).

### Example 92

### Azepan-1-yl-[3-(3-dimethylamino-pyrrolidin-1-ylmethyl)-1H-indol-6-yl]-methanone

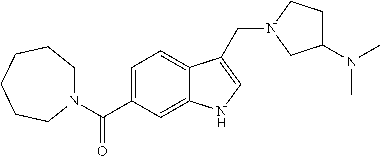

LC/MS: Rₜ=8.30. MS (ESI): mass calcd. for C₂₂H₃₂N₄O, 368.5; m/z found, 369.1 [M+H]⁺. ¹H NMR (CDCl₃): 8.35 (s, 1H), 7.72 (d, J=8.0, 1H), 7.44 (s, 1H), 7.21 (d, J=2.0, 1H), 7.14 (dd, J=8.5, 1.5, 1H), 3.82 (q, J=13.5, 4.5, 2H), 3.75-3.69 (m, 2H), 3.49-3.43 (m, 2H), 2.92 (t, J=7.0, 1H), 2.83-2.74 (m, 2H), 2.58-2.54 (m, 1H), 2.37 (t, J=7.5, 1H), 2.21 (s, 6H), 2.04-1.97 (m, 1H), 1.91-1.86 (m, 2H), 1.76-1.57 (m, 7H).

### Example 93

### Azepan-1-yl-[3-(4-cyclobutyl-piperazin-1-ylmethyl)-1H-indol-6-yl]-methanone

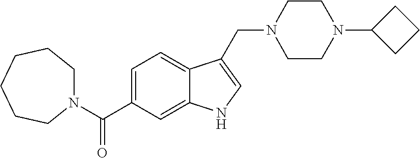

LC/MS: Rₜ=9.05. MS (ESI): mass calcd. for C₂₄H₃₄N₄O, 394.6; m/z found, 395.1 [M+H]⁺. ¹H NMR (CDCl₃): 8.32 (s, 1H), 7.73 (d, J=8.0, 1H), 7.44 (s, 1H), 7.20 (d, J=2.5, 1H), 7.14 (dd, J=8.5, 1.5, 1H), 3.75 (s, 2H), 3.74-3.70 (m, 2H), 3.49-3.44 (m, 2H), 2.76-2.70 (m, 1H), 2.68-2.09 (m, 7H), 2.05-1.99 (m, 2H), 1.91-1.83 (m, 4H), 1.73-1.58 (m, 9H).

### Example 94

### [3-(3-Dimethylamino-pyrrolidin-1-ylmethyl)-1H-indol-6-yl]-(4-hydroxymethyl-piperidin-1-yl)-methanone

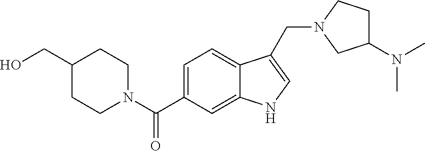

LC/MS: Rₜ=15.00. MS (ESI): mass calcd. for C₂₂H₃₂N₄O₂, 384.5; m/z found, 385.2 [M+H]⁺.

### Example 95

### Azepan-1-yl-[3-(4-isopropyl-[1,4]diazepan-1-ylmethyl)-1H-indol-6-yl]-methanone

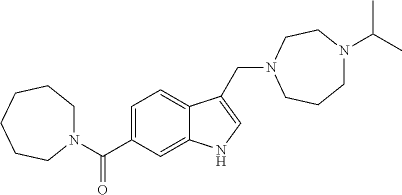

LC/MS: Rₜ=14.00. MS (ESI): mass calcd. for C₂₄H₃₆N₄O, 384.5; m/z found, 385.2 [M+H]⁺.

### Example 96

### [3-(4-Dimethylamino-piperidin-1-ylmethyl)-1H-indol-6-yl]-(4-hydroxymethyl-piperidin-1-yl)-methanone

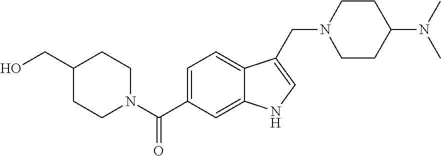

LC/MS: Rₜ=8.64. MS (ESI): mass calcd. for C₂₃H₃₄N₄O₂, 398.5; m/z found, 399.2 [M+H]⁺.

### Example 97

### [3-(4-Isopropyl-[1,4]diazepan-1-ylmethyl)-1H-indol-6-yl]-(4-phenyl-piperidin-1-yl)-methanone

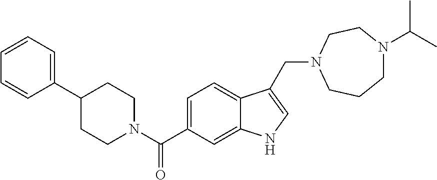

LC/MS: Rₜ=16.50. MS (ESI): mass calcd. for C₂₉H₃₈N₄O, 458.6; m/z found, 459.2 [M+H]⁺. ¹H NMR (CDCl₃): 8.36 (s, 1H), 7.78 (d, J=8.0, 1H), 7.53 (s, 1H), 7.35 (t, J=7.5, 2H), 7.26-7.23 (m, 3H), 7.22-7.19 (m, 2H), 3.84 (s, 2H), 3.22-2.94 (m, 2H), 2.92-2.87 (m, 1H), 2.87-2.84 (m, 1H), 2.77-2.65 (m, 8H), 2.07-1.85 (m, 2H), 1.85-1.72 (m, 6H), 1.01 (d, J=6.5, 6H).

### Example 98

### Azepan-1-yl-[3-(4-cyclobutyl-[1,4]diazepan-1-ylmethyl)-1H-indol-6-yl]-methanone

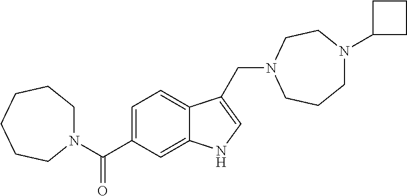

LC/MS: Rₜ=11.69. MS (ESI): mass calcd. for C₂₅H₃₆N₄O, 408.6; m/z found, 409.2 [M+H]⁺.

### Example 99

### (4-Benzyl-piperidin-1-yl)-[3-(4-cyclopentyl-piperazin-1-ylmethyl)-1H-indol-6-yl]-methanone

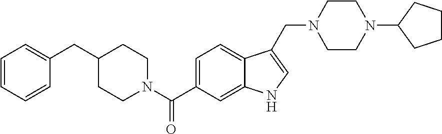

LC/MS: Rₜ=13.22. MS (ESI): mass calcd. for C₃₁H₄₀N₄O, 484.7; m/z found, 485.2 [M+H]⁺.

### Example 100

### (4-Benzyl-piperidin-1-yl)-[3-(4-dimethylamino-piperidin-1-ylmethyl)-1H-indol-6-yl]-methanone

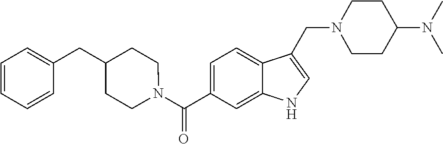

LC/MS: Rₜ=14.49. MS (ESI): mass calcd. for C₂₉H₃₈N₄O, 458.6; m/z found, 459.3 [M+H]⁺.

### Example 101

### (4-Benzyl-piperidin-1-yl)-[3-dimethylamino-pyrrolidin-1-ylmethyl)-1H-indol-6-yl]-methanone

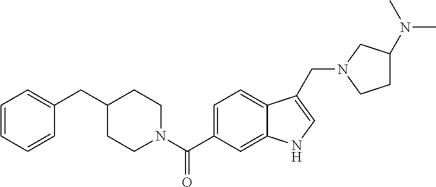

LC/MS: Rₜ=11.78. MS (ESI): mass calcd. for C₂₈H₃₆N₄O, 444.6; m/z found, 445.2 [M+H]⁺. ¹H NMR (CDCl₃): 8.44 (s, 1H), 7.71 (d, J=8.0, 1H), 7.48 (s, 1H), 7.30 (t, J=7.5, 2H), 7.23-7.20 (m, 2H), 7.17-7.13 (m, 3H), 3.84-3.78 (m, 2H), 2.97-2.73 (m, 2H), 2.92-2.89 (m, 1H), 2.82-2.73 (m, 3H), 2.60-2.53 (m, 3H), 2.36 (t, J=7.5, 1H), 2.20 (s, 6H), 2.03-1.96 (m, 1H), 1.86-1.71 (m, 5H), 1.36-1.19 (m, 2H).

### Example 102

### (4-Isopropyl-piperazin-1-yl)-(3-piperidin-1-ylmethyl-benzo[b]thiophen-5-yl)-methanone

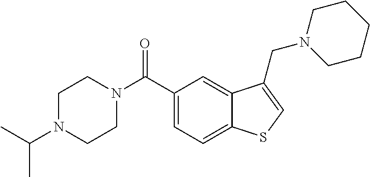

Step A: (5-Bromo-benzo[b]thiophen-3-yl)-methanol. To a 0° C. solution of 5-bromo-benzo[b]thiophene-3-carboxylic acid (500 mg, 1.9 mmol) and TEA (236 mg, 2.3 mmol) in THF (20 mL) was added iso-butylchloroformate (314 mg, 2.3 mmol) and the mixture was stirred at 0° C. for 2 h. The suspension was filtered and the organic layer was partially concentrated and cooled to 0° C. The solution was treated with NaBH₄ (148 mg, 2.0 mmol) and the resulting suspension was treated with H₂O (10 mL) over 30 min and warmed to rt over 14 h. The suspension was partially concentrated and the resulting suspension was partitioned between EtOAc and 1 N NaOH (50 mL). The organic layer was washed with brine (50 mL), dried, and concentrated to provide 0.42 g (89%) of the title compound as a white solid. ¹H NMR (CDCl₃): 8.02 (d, J=1.8, 1H), 7.72 (d, J=8.6, 1H), 7.48-7.43 (m, 2H), 4.91 (d, J=0.5, 2H).

Step B: (3-Hydroxymethyl-benzo[b]thiophen-5-yl)-(4-isopropyl-piperazin-1-yl)-methanone. To a suspension of (5-bromo-benzo[b]thiophen-3-yl)-methanol (130 mg, 0.53 mmol), 1-isopropyl-piperazine (205 mg, 1.6 mmol), 1,8-diazabicyclo[5.4.0]undec-7-ene (DBU; 244 mg, 1.6 mmol), tBu₃PBF₄ (15 mg, 0.05 mmol) and trans-di-m-acetatobis[2-(di-o-tolylphosphino)benzyl]di-palladium (II) (Hermann's catalyst; 25 mg, 0.03 mmol) in THF was added Mo(CO)₆ (141 mg, 0.53 mmol) and the reaction mixture was sealed and heated at 125° C. with microwave irradiation for 8 min. The solution was concentrated and the resulting residue was partitioned between EtOAc and 1 N NaOH (25 mL). The organic layer was washed with brine (250 mL), dried, and concentrated. The resulting residue was purified by FCC to provide 30 mg (18%) of the title compound as a colorless oil. MS (ESI): mass calcd. for C₁₇H₂₂N₂O₂S, 318.44; m/z found, 319.2 [M+H]⁺. ¹H NMR (CDCl₃): 7.88-7.84 (m, 2H), 7.42 (s, 1H), 7.39-7.35 (m, 1H), 4.88 (s, 2H), 3.82 (br s, 2H), 3.46 (br s, 2H), 2.73 (h, J=6.4, 1H), 2.51 (br s, 2H), 2.36 (br s, 2H), 1.06 (d, J=6.4, 6H).

Step C: 5-(4-Isopropyl-piperazine-1-carbonyl)-benzo[b]thiophene-3-carbaldehyde. To a solution of (3-hydroxymethyl-benzo[b]thiophen-5-yl)-(4-isopropyl-piperazin-1-yl)-methanone (80 mg, 0.25 mmol) in CHCl₃ (4 mL) was added MnO₂ (219 mg, 2.5 mmol). The mixture was heated at 70° C. for 2 h. The suspension was filtered through a pad of diatomaceous earth and the filtrate was concentrated to provide 75 mg (95%) of the title compound as a colorless oil. MS (ESI): mass calcd. for C₁₇H₂₀N₂O₂S, 316.43; m/z found, 317.1 [M+H]⁺. ¹H NMR (CDCl₃): 10.14 (s, 1H), 8.73 (d, J=0.9, 1H), 8.38 (s, 1H), 7.92 (d, J=8.3, 1H), 7.52 (dd, J=8.4, 1.4, 1H), 3.83 (br s, 2H), 3.46 (br s, 2H), 2.73 (h, J=6.4, 1H), 2.48 (br s, 2H), 2.35 (br s, 2H), 1.09 (d, J=6.4, 6H).

Step D: (4-Isopropyl-piperazin-1-yl)-(3-piperidin-1-ylmethyl-benzo[b]thiophen-5-yl)-methanone. To a solution of 5-(4-isopropyl-piperazine-1-carbonyl)-benzo[b]thiophene-3-carbaldehyde (65 mg, 0.21 mmol) and piperidine (18 mg, 0.21 mmol) in DCM (2 mL) was added NaBH(OAc)₃ (110 mg, 0.51 mmol). After 24 h, the solution was concentrated and the resulting residue was partitioned between EtOAc and 1 N NaOH (20 mL). The organic layer was washed with brine (25 mL), dried, and concentrated. The resulting residue was purified by FCC to provide 47 mg (59%) of the title compound as a colorless oil. LC/MS: Rₜ=3.65. MS (ESI): mass calcd. for C₂₂H₃₁N₃OS, 385.58; m/z found, 386.2 [M+H]⁺. ¹H NMR (CDCl₃): 8.07 (d, J=1.0, 1H), 7.84 (d, J=8.3, 1H), 7.39 (dd, J=8.3, 1.5, 1H), 7.31 (5, 1H), 3.83 (br s, 2H), 3.68 (5, 2H), 3.50 (br s, 2H), 2.71 (h, J=6.4, 1H), 2.61-2.41 (m, 8H), 1.56-1.52 (m, 4H), 1.43 (br s, 2H), 1.05 (d, J=6.4, 6H).

### Example 103

### (4-Cyclobutyl-piperazin-1-yl)-(3-piperidin-1-ylmethyl-benzo[b]thiophen-5-yl)-methanone

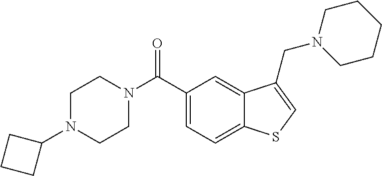

The title compound was prepared using methods analogous to those described for Example 102. LC/MS: Rₜ=3.67. MS (ESI): mass calcd. for C₂₃H₃₁N₃OS, 397.59; m/z found, 398.2 [M+H]⁺. ¹H NMR (CDCl₃): 8.06 (d, J=1.5, 1H), 7.85 (d, J=8.0, 1H), 7.39 (dd, J=8.0, 1.5, 1H), 7.35 (br s, 1H), 3.84 (br s, 2H), 3.70 (5, 2H), 3.51 (br s, 2H), 2.76 (h, J=7.9, 1H), 2.42 (br s, 6H), 2.29 (br s, 2H), 2.07-2.01 (m, 2H), 1.90-1.84 (m, 2H), 1.74-1.68 (m, 2H), 1.57-1.54 (m, 4H), 1.43 (br s, 2H).

### Example 104

### [5-(4-Isopropyl-piperazine-1-carbonyl)-benzo[b]thiophen-3-yl]-piperidin-1-yl-methanone

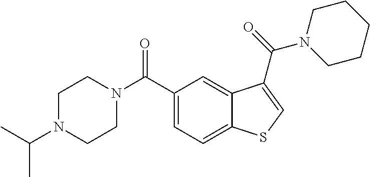

Step A: (5-Bromo-benzo[b]thiophen-3-yl)-piperidin-1-yl-methanone. To a solution of 5-bromo-benzo[b]thiophene-3-carboxylic acid (1.0 g, 3.9 mmol), piperidine (0.33 g, 3.9 mmol) and HOBt (0.8 g, 5.8 mmol) in DMF (39 mL) was added EDC (1.1 g, 5.8 mmol). After 24 h, the solution was partitioned between EtOAc and 1 N NaOH (200 mL). The organic layer was washed with brine (200 mL), dried, and concentrated. The resulting residue was purified by FCC to provide 1.23 g (98%) of the title compound as a tan solid. MS (ESI): mass calcd. for C₁₄H₁₄BrNOS, 324.24; m/z found, 324.0 [M]⁺. ¹H NMR (CDCl₃): 7.99 (d, J=1.9, 1H), 7.72 (d, J=8.6, 1H), 7.54 (s, 1H), 7.47 (dd, J=8.6, 1.9, 1H), 3.61 (br s, 2H), 3.28 (br s, 2H), 1.70 (br s, 4H), 1.57 (br s, 2H).

Step B: [5-(4-Isopropyl-piperazine-1-carbonyl)-benzo[b]thiophen-3-yl]-piperidin-1-yl-methanone. To a suspension of (5-bromo-benzo[b]thiophen-3-yl)-piperidin-1-yl-methanone (370 mg, 1.2 mmol), 1-isopropyl-piperazine (147 mg, 1.2 mmol), Na₂CO₃ (607 mg, 5.7 mmol), and Hermann's catalyst (54 mg, 0.06 mmol) in H₂O (2 mL) was added Mo(CO)₆ (151 mg, 0.57 mmol) and the reaction mixture was sealed and heated at 130° C. with microwave irradiation for 10 min. The solution was concentrated and the resulting residue was partitioned between EtOAc and 1 N NaOH (50 mL). The organic layer was washed with brine (50 mL), dried, and concentrated. The resulting residue was purified by FCC to provide 193 mg (41%) of the title compound as a tan foam. LC/MS: Rₜ=4.43. MS (ESI): mass calcd. for C₂₂H₂₉N₃O₂S, 399.56; m/z found, 400.2 [M+H]⁺. ¹H NMR (CDCl₃): 7.87 (d, J=8.3, 1H), 7.86 (s, 1H), 7.57 (s, 1H), 7.38 (d, J=8.3, 1H), 3.79 (br s, 4H), 3.59 (br s, 2H), 3.55 (br s, 2H), 2.71 (h, J=6.5, 1H), 2.58 (br s, 2H), 2.42 (br s, 2H), 1.66 (br s, 4H), 1.56 (br s, 2H), 1.02 (d, J=6.5, 6H).

### Example 105

### [5-(4-Cyclobutyl-piperazine-1-carbonyl)-benzo[b]thiophen-3-yl]-piperidin-1-yl-methanone

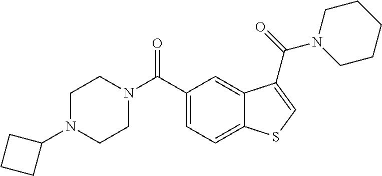

The title compound was prepared using methods analogous to those described for Example 104. LC/MS: Rₜ=4.37. MS (ESI): mass calcd. for C₂₃H₂₉N₃O₂S, 411.57; m/z found, 412.2 [M+H]⁺. ¹H NMR (CDCl₃): 7.86 (d, J=8.5, 1H), 7.85 (s, 1H), 7.56 (s, 1H), 7.37 (d, J=8.5, 1H), 3.79 (br s, 4H), 3.42 (br s, 4H), 2.72 (h, J=8.0, 1H), 2.38 (br s, 2H), 2.22 (br s, 2H), 2.03-1.98 (m, 2H), 1.88-1.81 (m, 2H), 1.73-1.56 (m, 8H).

The compounds in Examples 106-108 were prepared using methods analogous to those described in the preceding examples.

### Example 106

### (4-Benzyl-piperidin-1-yl)-(3-[1,4′]bipiperidinyl-1′-ylmethyl-1H-indol-6-yl)-methanone

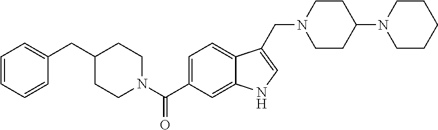

LC/MS: Rₜ=15.52. MS (ESI): mass calcd. for C₃₂H₄₂N₄O, 498.7; m/z found, 499.3 [M+H]⁺.

### Example 107

### (4-Benzyl-piperidin-1-yl)-[3-(4-cyclobutyl-piperazin-1-ylmethyl)-1H-indol-6-yl]-methanone

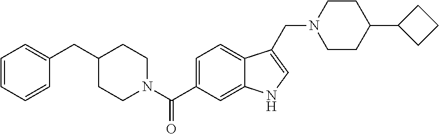

LC/MS: Rₜ=12.66. MS (ESI): mass calcd. for C₃₀H₃₈N₄O, 470.6; m/z found, 471.2 [M+H]⁺.

### Example 108

### (4-Benzyl-piperidin-1-yl)-[3-(4-cyclobutyl-[1,4]diazepan-1-ylmethyl)-1H-indol-6-yl]-methanone

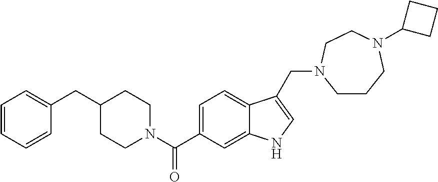

LC/MS: Rₜ=15.00. MS (ESI): mass calcd. for C₃₁H₄₀N₄O, 484.7; m/z found, 485.2 [M+H]⁺. ¹H NMR (CDCl₃): 8.28 (5, 1H), 7.76 (d, J=8.0, 1H), 7.48 (5, 1H), 7.31 (t, J=7.0, 2H), 7.23-7.20 (m, 2H), 7.17-7.14 (m, 3H), 3.83 (5, 2H), 2.99-2.68 (m, 3H), 2.96-2.90 (m, 1H), 2.78 (t, J=6.0, 2H), 2.74-2.72 (m, 2H), 2.61-2.57 (m, 5H), 2.52-2.50 (m, 2H), 2.04-1.99 (m, 2H), 1.88-1.77 (m, 5H), 1.71-1.58 (m, 4H), 1.37-1.20 (m, 2H).

### Biological Methods:

### H₃ Receptor Binding (Human)

Binding of compounds to the cloned human H₃ receptors, stably expressed in SK-N-MC cells, was performed as described by Barbier, A. J. et al. (Br. J. Pharmacol. 2004, 143(5), 649-661). Data for compounds tested in this assay are presented in Table 1 as an average of the results obtained.

TABLE 1

| | Human H₃ |
|---|---|
| Ex. | Kᵢ (nM) |
| 1 | 2 |
| 2 | 46 |
| 3 | 1 |
| 4 | 1 |
| 5 | 1 |
| 6 | 1 |
| 7 | 1 |
| 8 | 1 |
| 9 | 1 |
| 10 | 5011 |
| 11 | 228 |
| 12 | 1995 |
| 13 | 1995 |
| 14 | 609 |
| 15 | 117 |
| 16 | 327 |
| 17 | 114 |
| 18 | 5011 |
| 19 | 260 |
| 20 | 10 |
| 21 | 5 |
| 22 | 32 |
| 23 | 2 |
| 24 | 2 |
| 25 | 3 |
| 26 | 18 |
| 27 | 28 |
| 28 | 177 |
| 29 | 11 |
| 30 | 26 |
| 31 | 214 |
| 32 | 34 |
| 33 | 13 |
| 34 | 4 |
| 35 | 265 |
| 36 | 81 |
| 37 | 318 |
| 38 | 427 |
| 39 | >10000 |
| 40 | 33 |
| 41 | 5000 |
| 42 | 1500 |
| 43 | 8 |
| 44 | 2 |
| 45 | 2 |
| 46 | 3 |
| 47 | 1 |
| 48 | 5 |
| 49 | 7 |
| 50 | 3 |
| 51 | 2 |
| 52 | 12 |
| 53 | 19 |
| 54 | 12 |
| 55 | 1 |
| 56 | 2 |
| 57 | 1 |
| 58 | 0 |
| 59 | 1 |
| 60 | 1 |
| 61 | 3 |
| 62 | 7 |
| 63 | 1 |
| 64 | 1 |
| 65 | 1 |
| 66 | 1256 |
| 67 | 171 |
| 68 | 36 |
| 69 | 19 |
| 70 | 27 |
| 71 | 22 |
| 72 | 36 |
| 73 | 205 |
| 74 | 53 |
| 75 | 43 |
| 76 | 54 |
| 77 | 38 |
| 78 | 42 |
| 79 | 62 |
| 80 | 38 |
| 81 | 17 |
| 82 | 34 |
| 83 | 27 |
| 84 | 87 |
| 85 | 81 |
| 86 | 27 |
| 87 | 20 |
| 88 | 186 |
| 89 | 31 |
| 90 | 21 |
| 91 | 26 |
| 92 | 15 |
| 93 | 38 |
| 94 | 27 |
| 95 | 42 |
| 96 | 439 |
| 97 | 59 |
| 98 | 34 |
| 99 | 28 |
| 100 | 233 |
| 101 | 9 |
| 102 | 17 |
| 103 | 7 |
| 104 | 772 |
| 105 | 215 |
| 106 | 70 |
| 107 | 12 |
| 108 | 16 |

### H₃ Receptor Binding (Rat)

A rat brain without cerebellum (Zivic Laboratories Inc., Pittsburgh, Pa.) was homogenized in 50 mM Tris-HCl/5 mM EDTA and centrifuged at 1,000 rpm for 5 min. The supernatant was removed and recentrifuged at 15,000 rpm for 30 min. Pellets were rehomogenized in 50 mM Tris/5 mM EDTA (pH 7.4). Membranes were incubated with 0.8 nM N—[³H]-α-methylhistamine plus/minus test compounds for 60 min at 25° C. and harvested by rapid filtration over GF/C glass fiber filters (pretreated with 0.3% polyethylenimine) followed by four washes with buffer. Nonspecific binding was defined in the presence of 100 μM histamine. Inhibitory concentration (responsible for 50% inhibition of maximal effect, IC₅₀) values were determined by a single site curve-fitting program (GraphPad, San Diego, Calif.) and converted to Kᵢ values based on a N—[³H]-α-methylhistamine dissociation constant (K_{d}) of 0.8 nM. Data for compounds tested in this assay are presented in Table 2 as an average of the results obtained.

TABLE 2

| | Rat H₃ |
|---|---|
| Ex. | Kᵢ (nM) |
| 1 | 98 |
| 4 | 14 |
| 5 | 28 |
| 7 | 2 |
| 21 | 19 |
| 29 | 438 |
| 34 | 133 |
| 58 | 2 |
| 102 | 228 |

### Cyclic AMP Accumulation

Sublines of SK-N-MC cells were created that expressed a reporter construct and either the human or rat H₃ receptor. The pA₂ values were obtained as described by Barbier et al. (2004). Data for compounds tested in these assays are presented in Table 3, as an average of the results obtained (NT=not tested).

TABLE 3

| Ex. | Human pA₂ | Rat pA₂ |
|---|---|---|
| 1 | 8.43 | NT |
| 3 | 9.30 | NT |
| 4 | 9.63 | NT |
| 5 | 9.27 | NT |
| 6 | 9.81 | NT |
| 7 | NT | 8.70 |
| 20 | 8.09 | NT |
| 21 | 8.96 | NT |
| 23 | 9.24 | NT |
| 25 | 8.51 | NT |
| 44 | 8.97 | NT |
| 45 | 8.74 | NT |
| 47 | 9.60 | NT |
| 57 | 8.93 | NT |
| 91 | 7.33 | 7.17 |

What is claimed is:

1. A compound of Formula (I): 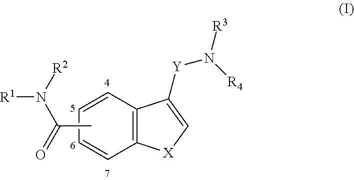
wherein
X is NR^{a} and Y is —CH₂— or X is S and Y is —CH₂— or —C(O)—;
where R^{a} is —H, methyl, —SO₂methyl;
the substituent —C(O)NR¹R² is bound at the 4-, 5-, 6-, or 7-position on Formula (I);
R¹ is —H and R² is —(CH₂)-pyridyl, where said pyridyl is unsubstituted or substituted with methyl;
or R¹ and R² taken together with the nitrogen to which they are attached form one of the following moieties: 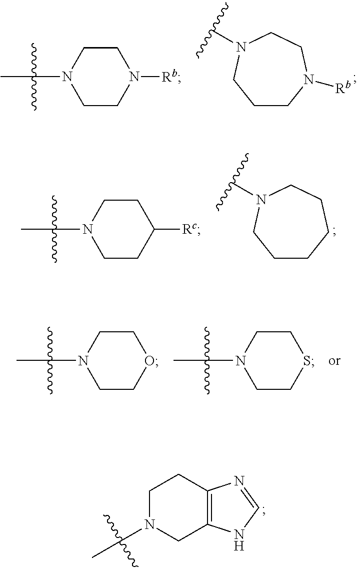
where R^{b} is isopropyl, cyclopropyl, or cyclobutyl; and
R^{c} is —H, hydroxymethyl, phenyl, or 1-pyrrolidin-2-onyl;
R³ and R⁴ taken together with the nitrogen to which they are attached form one of the following moieties: 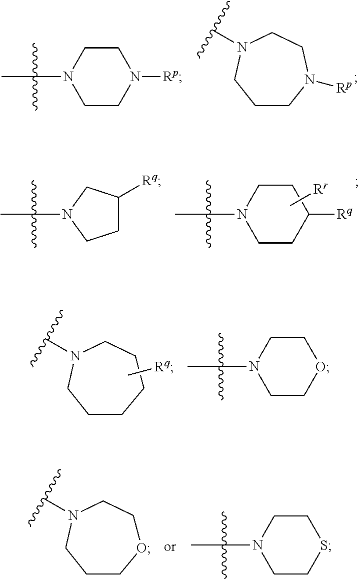
where R^{p} is isopropyl, acetyl, methylsulfonyl, C₃₋₅cycloalkyl, phenyl, —C(O)-phenyl, biphenyl, benzyl, benzhydryl, phenethyl, pyridyl, —C(O)-pyridyl, thiazolyl, or —C(O)-morpholinyl;
R^{q} is —H, —OH, phenyl, benzyl, —NR^{s}R^{t}, or —N(R^{s})C(O)R^{t};
where R^{s} and R^{t} are each independently —H or methyl;
or alternatively, R^{s} and R^{t} taken together with the nitrogen to which they are attached form piperidine; and
R^{r} is —H or —OH;
with the following provisos:
1) when
a) the substituent —C(O)NR¹R² is bound at the 5-position in Formula (I); and
b) R¹ and R² taken together with the nitrogen to which they are attached form one of the following moieties: 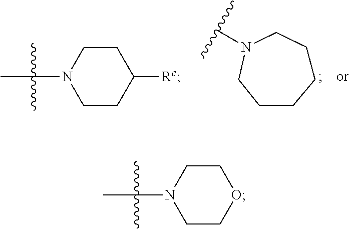
and
c) R^{c} is —H;
then R³ and R⁴ taken together with the nitrogen to which they are attached do not form one of the following moieties: 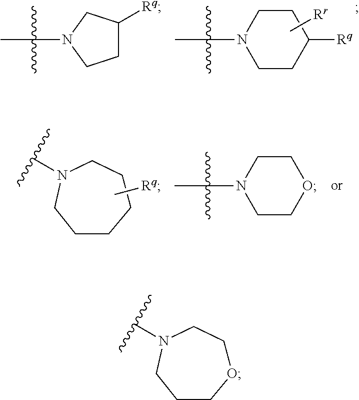
where R^{q} is —H and R^{r} is —H;
2) when
a) X is NR^{a}; and
b) the substituent —C(O)NR¹R² is bound at the 4- or 7-position on Formula (I);
then the substituents —C(O)NR¹R² and —YNR³R⁴ together comprise two nitrogens each of which is not adjacent to a carbonyl or sulfonyl group;
3) when
a) NR¹R² is 4-benzylpiperidin-1-yl; and
b) the substituent —C(O)NR¹R² is bound at the 5- or 6-position on Formula (I);
then R³ and R⁴ taken together with the nitrogen to which they are attached do not form one of the following moieties: 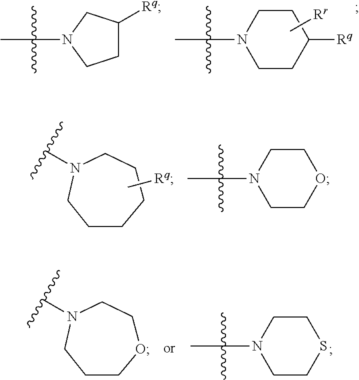
where R^{q} is —H and R^{r} is —H;
or a pharmaceutically acceptable salt, a pharmaceutically acceptable prodrug, or a pharmaceutically active metabolite thereof.

2. A compound as defined in claim 1, wherein X is NR^{a} and Y is —CH₂—.

3. A compound as defined in claim 1, wherein X is S and Y is —C(O)—.

4. A compound as defined in claim 1, wherein R^{a} is —H.

5. A compound as defined in claim 1, wherein the substituent —C(O)NR¹R² is bound at the 5- or 6-position on Formula (I).

6. A compound as defined in claim 1, wherein the substituent —C(O)NR¹R² is bound at the 6-position on Formula (I).

7. A compound as defined in claim 1, wherein R¹ is —H and R² is pyridin-3-ylmethyl, pyridin-4-ylmethyl, or 3-methyl-pyridin-2-ylmethyl.

8. A compound as defined in claim 1, wherein R¹ and R² taken together with the nitrogen to which they are attached form one of the following moieties: 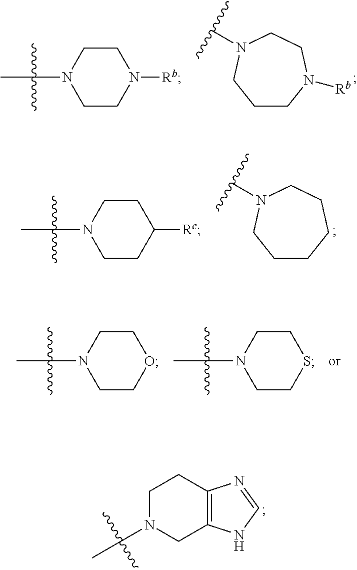
where R^{b} is cyclopropyl or cyclobutyl; and
R^{c} is hydroxymethyl, phenyl, or 1-pyrrolidin-2-onyl.

9. A compound as defined in claim 1, wherein R¹ and R² taken together with the nitrogen to which they are attached form

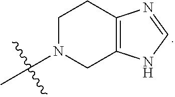

10. A compound as defined in claim 8, wherein R³ and R⁴ taken together with the nitrogen to which they are attached form one of the following moieties: 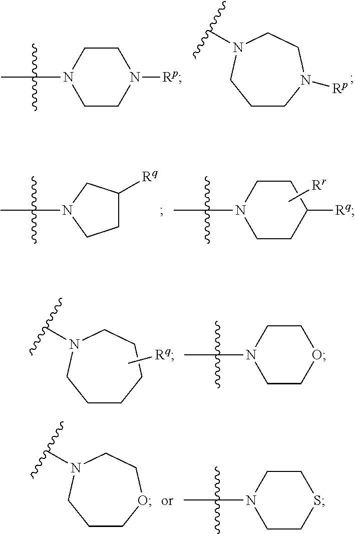
where R^{p}, R^{q}, and R^{r} are as defined in Formula (I).

11. A compound as defined in claim 9, wherein R³ and R⁴ taken together with the nitrogen to which they are attached form one of the following moieties: 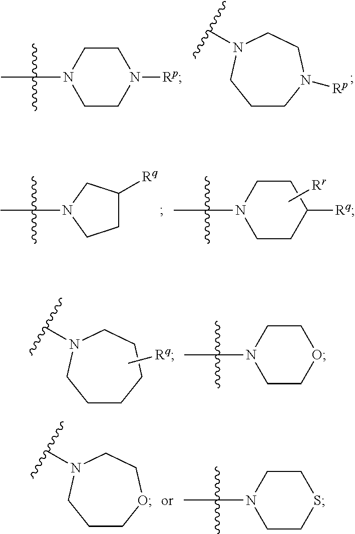
where R^{p}, R^{q}, and R^{r} are as defined in Formula (I).

12. A compound as defined in claim 1, wherein R³ and R⁴ taken together with the nitrogen to which they are attached form one of the following moieties: 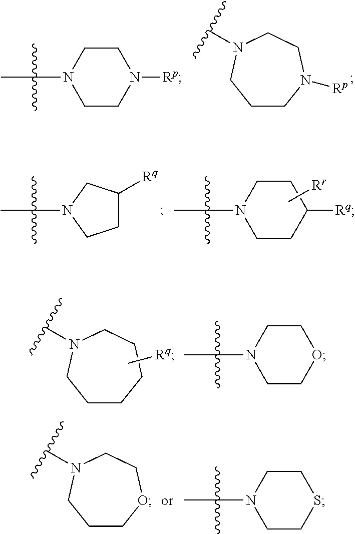
where R^{q} is —OH, phenyl, benzyl, —NR^{s}R^{t}, or —N(R^{s})C(O)R^{t};
and R^{p}, R^{r}, R^{s} and R^{t} are defined as in Formula (I).

13. A compound as defined in claim 1, wherein R^{p} is isopropyl, cyclopropyl, or cyclobutyl.

14. A compound as defined in claim 1, wherein R^{q} is —H.

15. A compound selected from the group consisting of:
(4-Isopropyl-piperazin-1-yl)-(1-methyl-3-morpholin-4-ylmethyl-1H-indol-6-yl)-methanone;
(4-Cyclopropyl-piperazin-1-yl)-(1-methyl-3-morpholin-4-ylmethyl-1H-indol-6-yl)-methanone;
(4-Cyclobutyl-piperazin-1-yl)-(1-methyl-3-morpholin-4-ylmethyl-1H-indol-6-yl)-methanone;
(4-Isopropyl-piperazin-1-yl)-(3-morpholin-4-ylmethyl-1H-indol-6-yl)-methanone;
(4-Cyclopropyl-piperazin-1-yl)-(3-morpholin-4-ylmethyl-1H-indol-6-yl)-methanone;
(4-Cyclobutyl-piperazin-1-yl)-(3-morpholin-4-ylmethyl-1H-indol-6-yl)-methanone;
(4-Isopropyl-[1,4]diazepan-1-yl)-(3-morpholin-4-ylmethyl-1H-indol-6-yl)-methanone;
(4-Cyclopropyl-[1,4]diazepan-1-yl)-(3-morpholin-4-ylmethyl-1H-indol-6-yl)-methanone;
(4-Cyclobutyl-[1,4]diazepan-1-yl)-(3-morpholin-4-ylmethyl-1H-indol-6-yl)-methanone;
3-Morpholin-4-ylmethyl-1H-indole-6-carboxylic acid (pyridin-3-ylmethyl)-amide;
3-Morpholin-4-ylmethyl-1H-indole-6-carboxylic acid (pyridin-4-ylmethyl)-amide;
3-Morpholin-4-ylmethyl-1H-indole-6-carboxylic acid (3-methyl-pyridin-2-ylmethyl)-amide;
(3-Morpholin-4-ylmethyl-1H-indol-6-yl)-(3,4,6,7-tetrahydro-imidazo[4,5-c]pyridin-5-yl)-methanone;
1-[1-(3-Morpholin-4-ylmethyl-1H-indole-6-carbonyl)-piperidin-4-yl]-pyrrolidin-2-one;
3-Piperidin-1-ylmethyl-1H-indole-6-carboxylic acid (pyridin-3-ylmethyl)-amide;
3-Piperidin-1-ylmethyl-1H-indole-6-carboxylic acid (pyridin-4-ylmethyl)-amide;
3-Piperidin-1-ylmethyl-1H-indole-6-carboxylic acid (3-methyl-pyridin-2-ylmethyl)-amide;
(3-Piperidin-1-ylmethyl-1H-indol-6-yl)-(3,4,6,7-tetrahydro-imidazo[4,5-c]pyridin-5-yl)-methanone;
1-[1-(3-Piperidin-1-ylmethyl-1H-indole-6-carbonyl)-piperidin-4-yl]-pyrrolidin-2-one;
(4-Isopropyl-piperazin-1-yl)-(3-morpholin-4-ylmethyl-1H-indol-7-yl)-methanone;
(4-Cyclobutyl-piperazin-1-yl)-(3-morpholin-4-ylmethyl-1H-indol-7-yl)-methanone;
(4-Isopropyl-piperazin-1-yl)-(3-piperidin-1-ylmethyl-1H-indol-7-yl)-methanone;
(4-Cyclobutyl-piperazin-1-yl)-(3-piperidin-1-ylmethyl-1H-indol-7-yl)-methanone;
(4-Isopropyl-piperazin-1-yl)-(3-piperidin-1-ylmethyl-1H-indol-6-yl)-methanone;
(4-Isopropyl-piperazin-1-yl)-(1-methanesulfonyl-3-piperidin-1-ylmethyl-1H-indol-6-yl)-methanone;
(4-Isopropyl-piperazin-1-yl)-(1-methanesulfonyl-3-morpholin-4-ylmethyl-1H-indol-6-yl)-methanone;
[3-(4-Isopropyl-piperazin-1-ylmethyl)-1H-indol-6-yl]-piperidin-1-yl-methanone;
[3-(4-Cyclopropyl-piperazin-1-ylmethyl)-1H-indol-6-yl]-piperidin-1-yl-methanone;
[3-(4-Cyclobutyl-piperazin-1-ylmethyl)-1H-indol-6-yl]-piperidin-1-yl-methanone;
[3-(4-Isopropyl-[1,4]diazepan-1-ylmethyl)-1H-indol-6-yl]-piperidin-1-yl-methanone;
[3-(4-Cyclopropyl-[1,4]diazepan-1-ylmethyl)-1H-indol-6-yl]-piperidin-1-yl-methanone;
(4-Isopropyl-piperazin-1-yl)-(3-piperidin-1-ylmethyl-1H-indol-5-yl)-methanone;
(4-Cyclobutyl-piperazin-1-yl)-(3-piperidin-1-ylmethyl-1H-indol-5-yl)-methanone;
(4-Isopropyl-[1,4]diazepan-1-yl)-(3-piperidin-1-ylmethyl-1H-indol-5-yl)-methanone;
(4-Isopropyl-piperazin-1-yl)-(3-morpholin-4-ylmethyl-1H-indol-5-yl)-methanone;
[3-(4-Cyclobutyl-piperazin-1-ylmethyl)-1H-indol-7-yl]-piperidin-1-yl-methanone;
[3-(4-Cyclobutyl-piperazin-1-ylmethyl)-1H-indol-4-yl]-piperidin-1-yl-methanone;
[3-(4-Cyclobutyl-piperazin-1-ylmethyl)-1H-indol-5-yl]-piperidin-1-yl-methanone;
(4-Isopropyl-piperazin-1-yl)-(3-morpholin-4-ylmethyl-1H-indol-4-yl)-methanone;
(4-Cyclobutyl-piperazin-1-yl)-(3-morpholin-4-ylmethyl-1H-indol-5-yl)-methanone;
(4-Cyclobutyl-piperazin-1-yl)-(3-morpholin-4-ylmethyl-1H-indol-4-yl)-methanone;
(4-Cyclobutyl-piperazin-1-yl)-(3-piperidin-1-ylmethyl-1H-indol-4-yl)-methanone;
[3-(4-Cyclobutyl-piperazine-1-ylmethyl)-1H-indol-6-yl]-(4-phenyl-piperidin-1-yl)-methanone;
[3-(3-Hydroxymethyl-piperidin-1-ylmethyl)-1H-indol-6-yl]-(4-isopropyl-piperazin-1-yl)-methanone;
(4-Isopropyl-piperazin-1-yl)-[3-(4-phenyl-piperazin-1-ylmethyl)-1H-indol-6-yl]-methanone;
(4-Isopropyl-piperazin-1-yl)-[3-(4-pyridin-2-yl-piperazin-1-ylmethyl)-1H-indol-6-yl]-methanone;
(4-Isopropyl-piperazin-1-yl)-(3-thiomorpholin-4-ylmethyl-1H-indol-6-yl)-methanone;
1-{4-[6-(4-Isopropyl-piperazine-1-carbonyl)-1H-indol-3-ylmethyl]-piperazin-1-yl}-ethanone;
(4-Isopropyl-piperazin-1-yl)-[3-(4-thiazol-2-yl-piperazin-1-ylmethyl)-1H-indol-6-yl]-methanone;
1-{4-[6-(4-Isopropyl-piperazine-1-carbonyl)-1H-indol-3-ylmethyl]-[1,4]diazepam-1-yl}ethanone;
[3-(4-Benzyl-piperazin-1-ylmethyl)-1H-indol-6-yl]-(4-isopropyl-piperazin-1-yl)-methanone;
[3-(4-Biphenyl-4-yl-piperazin-1-ylmethyl)-1H-indol-6-yl]-(4-isopropyl-piperazin-1-yl)-methanone;
[3-(4-Benzydryl-piperazin-1-ylmethyl)-1H-indol-6-yl]-(4-isopropyl-piperazin-1-yl)-methanone;
(4-Isopropyl-piperazin-1-yl)-[3-(4-methanesulfonyl-piperazin-1-ylmethyl)-1H-indol-6-yl]-methanone;
[3-(4-Benzyl-piperidin-1-ylmethyl)-1H-indol-6-yl]-(4-isopropyl-piperazin-1-yl)-methanone;
(4-Isopropyl-piperazin-1-yl)-[3-(4-phenethyl-piperazin-1-ylmethyl)-1H-indol-6-yl]-methanone;
(4-Isopropyl-piperazin-1-yl)-[3-(4-phenyl-piperidin-1-ylmethyl)-1H-indol-6-yl]-methanone;
(4-Isopropyl-piperazin-1-yl)-(3-pyrrolidin-1-ylmethyl-1H-indol-6-yl)-methanone;
(4-Isopropyl-piperazin-1-yl)-(3-[1,4]-oxazepan-4-ylmethyl-1H-indol-6-yl)-methanone;
N-{1-[6-(4-Isopropyl-piperazine-1-carbonyl)-1H-indol-3-ylmethyl]-pyrrolidin-3-yl}-N-methyl-acetamide;
(4-Isopropyl-piperazin-1-yl)-{3-[4-(morpholine-4-carbonyl)-piperazin-1-ylmethyl]-1H-indol-6-yl}-methanone;
(4-Isopropyl-piperazin-1-yl)-{3-[4-(pyridine-4-carbonyl)-piperazin-1-ylmethyl]-1H-indol-6-yl}methanone;
[3-(4-Benzoyl-piperazin-1-ylmethyl)-1H-indol-6-yl]-(4-isopropyl-piperazin-1-yl)-methanone;
(4-Isopropyl-piperazin-1-yl)-[3-(4-pyridin-4-yl-piperazin-1-ylmethyl)-1H-indol-6-yl]-methanone;
[3-(4-Hydroxy-4-phenyl-piperidin-1-ylmethyl)-1H-indol-6-yl]-(4-isopropyl-piperazin-1-yl)-methanone;
(4-Isopropyl-piperazin-1-yl)-(3-thiomorpholin-4-ylmethyl-1H-indol-6-yl)-methanone;
(3-[1,4′]Bipiperidinyl-1′-ylmethyl-1H-indol-6-yl)-thiomorpholin-4-yl-methanone;
[3-(4-Cyclopentyl-piperazin-1-ylmethyl)-1H-indol-6-yl]-thiomorpholin-4-yl-methanone;
[3-(3-Dimethylamino-pyrrolidin-1-ylmethyl)-1H-indol-6-yl]-thiomorpholin-4-yl-methanone;
[3-(4-Cyclobutyl-piperazin-1-ylmethyl)-1H-indol-6-yl]-thiomorpholin-4-yl-methanone;
[3-(4-Cyclobutyl-[1,4]diazepan-1-ylmethyl)-1H-indol-6-yl]thiomorpholin-4-yl-methanone;
[3-(4-Isopropyl-[1,4]diazepan-1-ylmethyl)-1H-indol-6-yl]-thiomorpholin-4-yl-methanone;
(3-[1,4′]Bipiperidinyl-1′-ylmethyl-1H-indol-6-yl)-morpholin-4-yl-methanone;
[3-(4-Cyclopentyl-piperazin-1-ylmethyl)-1H-indol-6-yl]-morpholin-4-yl-methanone;
[3-(4-Cyclobutyl-piperazin-1-ylmethyl)-1H-indol-6-yl]-morpholin-4-yl-methanone;
[3-(4-Isopropyl-piperazin-1-ylmethyl)-1H-indol-6-yl]-morpholin-4-yl-methanone;
[3-(4-Cyclobutyl-[1,4]diazepan-1-ylmethyl)-1H-indol-6-yl]-morpholin-4-yl-methanone;
[3-(4-Isopropyl-[1,4]diazepan-1-ylmethyl)-1H-indol-6-yl]-morpholin-4-yl-methanone;
(3-[1,4′]Bipiperidinyl-1′-ylmethyl-1H-indol-6-yl)-(4-hydroxymethyl-piperidin-1-yl)-methanone;
[3-(4-Cyclopentyl-piperazin-1-ylmethy)l-1H-indol-6-yl]-(4-hydroxymethyl-piperidin-1-yl)-methanone;
[3-(4-Cyclobutyl-piperazin-1-ylmethy)l-1H-indol-6-yl]-(4-hydroxymethyl-piperidin-1-yl)-methanone;
(4-Hydroxymethyl-piperidin-1-yl)-[3-(4-isopropyl-piperazin-1-ylmethyl)-1H-indol-6-yl]-methanone;
[3-(4-Cyclobutyl-[1,4]diazepan-1-ylmethyl)-1H-indol-6-yl]-(4-hydroxymethyl-piperidin-1-yl)-methanone;
(4-Hydroxymethyl-piperidin-1-yl)-[3-(4-isopropyl-[1,4]diazepan-1-ylmethyl)-1H-indol-6-yl]-methanone;
(3-[1,4′]Bipiperidinyl-1′-ylmethyl-1H-indol-6-yl)-(4-phenyl-piperidin-1-yl)-methanone;
[3-(4-Cyclopentyl-piperazin-ylmethyl)-1H-indol-6-yl]-(4-phenyl-piperidin-1-yl)-methanone;
[3-(3-Dimethylamino-pyrrolidin-1-ylmethyl)-1H-indol-6-yl]-(4-phenyl-piperidin-1-yl)-methanone;
[3-(4-Dimethylamino-piperidin-1-ylmethyl)-1H-indol-6-yl]-(4-phenyl-piperidin-1-yl)-methanone;
[3-(4-Cyclobutyl-[1,4]diazepan-1-ylmethyl)-1H-indol-6-yl]-(4-phenyl-piperidin-1-yl)-methanone;
Azepan-1-yl-(3-[1,4]bipiperidinyl-1′-ylmethyl-1H-indol-6-yl)-methanone;
Azepan-1-yl-[3-(4-cyclopentyl-piperazin-1-ylmethyl)-1H-indol-6-yl]-methanone;
Azepan-1-yl-[3-(3-dimethylamino-pyrrolidin-1-ylmethyl)-1H-indol-6-yl]-methanone;
Azepan-1-yl-[3-(4-cyclobutyl-piperazin-1-ylmethyl)-1H-indol-6-yl]-methanone;
[3-(3-Dimethylamino-pyrrolidin-1-ylmethyl)-1H-indol-6-yl]-(4-hydroxymethyl-piperidin-1-yl)-methanone;
Azepan-1-yl-[3-(4-isopropyl-[1,4]diazepan-1-ylmethyl)-1H-indol-6-yl]-methanone;
[3-(4-Dimethylamino-piperidin-1-ylmethyl)-1H-indol-6-yl]-(4-hydroxymethyl-piperidin-1-yl)-methanone;
[3-(4-Isopropyl-[1,4]diazepan-1-ylmethyl)-1H-indol-6-yl]-(4-phenyl-piperidin-1-yl)-methanone;
Azepan-1-yl-[3-(4-cyclobutyl-[1,4]diazepan-1-ylmethyl)-1H-indol-6-yl]-methanone;
(4-Benzyl-piperidin-1-yl)-[3-(4-cyclopentyl-piperazin-1-ylmethyl)-1H-indol-6-yl]-methanone;
(4-Benzyl-piperidin-1-yl)-[3-(4-dimethylamino-piperidin-1-ylmethyl)-1H-indol-6-yl]-methanone;
(4-Benzyl-piperidin-1-yl)-[3-dimethylamino-pyrrolidin-1-ylmethyl)-1H-indol-6-yl]-methanone;
(4-Isopropyl-piperazin-1-yl)-(3-piperidin-1-ylmethyl-benzo[b]thiophen-5-yl)-methanone;
(4-Cyclobutyl-piperazin-1-yl)-(3-piperidin-1-ylmethyl-benzo[b]thiophen-5-yl)-methanone;
[5-(4-Isopropyl-piperazine-1-carbonyl)-benzo[b]thiophen-3-yl]-piperidin-1-yl-methanone;
[5-(4-Cyclobutyl-piperazine-1-carbonyl)-benzo[b]thiophen-3-yl]-piperidin-1-yl-methanone;
(4-Benzyl-piperidin-1-yl)-(3-[1,4′]bipiperidinyl-1′-ylmethyl-1H-indol-6-yl)-methanone;
(4-Benzyl-piperidin-1-yl)-[3-(4-cyclobutyl-piperazin-1-ylmethyl)-1H-indol-6-yl]-methanone; and
(4-Benzyl-piperidin-1-yl)-[3-(4-cyclobutyl-[1,4]diazepan-1-ylmethyl)-1H-indol-6-yl]-methanone;
and pharmaceutically acceptable salts thereof.

16. A compound as defined in claim 1, or a pharmaceutically acceptable salt thereof.

17. A pharmaceutical composition for treating a disease, disorder, or medical condition mediated by histamine H₃ receptor activity, comprising:
(a) an effective amount of a compound of Formula (I): 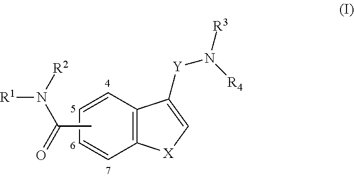
wherein
X is NR^{a} and Y is —CH₂— or X is S and Y is —CH₂— or —C(O)—;
where R^{a} is —H, methyl, —SO₂methyl;
the substituent —C(O)NR¹R² is bound at the 4-, 5-, 6-, or 7-position on Formula (I);
R¹ is —H and R² is —(CH₂)-pyridyl, where said pyridyl is unsubstituted or substituted with methyl;
or R¹ and R² taken together with the nitrogen to which they are attached form one of the following moieties: 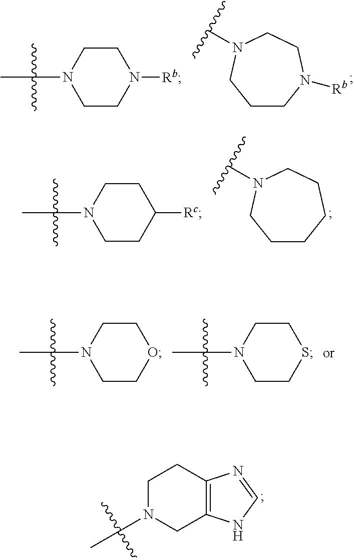
where R^{b} is isopropyl, cyclopropyl, or cyclobutyl; and
R^{c} is —H, hydroxymethyl, phenyl, or 1-pyrrolidin-2-onyl;
R³ and R⁴ taken together with the nitrogen to which they are attached form one of the following moieties: 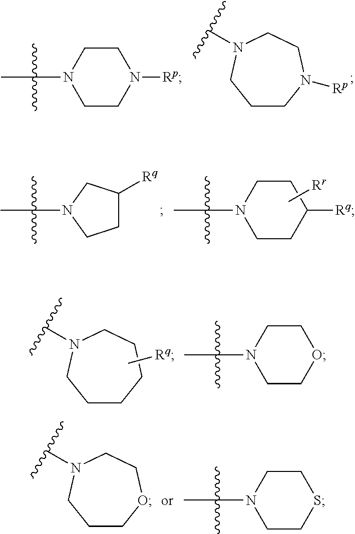
where R^{p} is isopropyl, acetyl, methylsulfonyl, C₃₋₅cycloalkyl, phenyl, —C(O)-phenyl, biphenyl, benzyl, benzhydryl, phenethyl, pyridyl, —C(O)-pyridyl, thiazolyl, or —C(O)-morpholinyl;
R^{q} is —H, —OH, phenyl, benzyl, —NR^{s}R^{t}, or —N(R^{s})C(O)R^{t};
where R^{s} and R^{t} are each independently —H or methyl;
or alternatively, R^{s} and R^{t} taken together with the nitrogen to which they are attached form piperidine; and
R^{r} is —H or —OH;
with the following provisos:
1) when
a) the substituent —C(O)NR¹R² is bound at the 5-position in Formula (I); and
b) R¹ and R² taken together with the nitrogen to which they are attached form one of the following moieties: 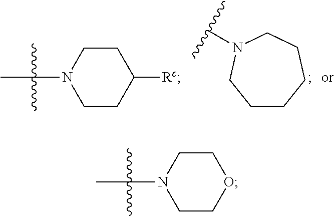
and
c) R^{c} is —H;
then R³ and R⁴ taken together with the nitrogen to which they are attached do not form one of the following moieties: 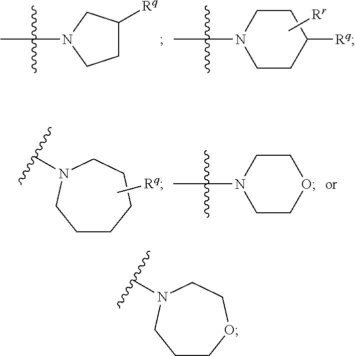
where R^{q} is —H and R^{r} is —H;
2) when
a) X is NR^{a}; and
b) the substituent —C(O)NR¹R² is bound at the 4- or 7-position on Formula (I);
then the substituents —C(O)NR¹R² and —YNR³R⁴ together comprise two nitrogens each of which is not adjacent to a carbonyl or sulfonyl group;
3) when
a) NR¹R² is 4-benzylpiperidin-1-yl; and
b) the substituent —C(O)NR¹R² is bound at the 5- or 6-position on Formula (I);
then R³ and R⁴ taken together with the nitrogen to which they are attached do not form one of the following moieties: 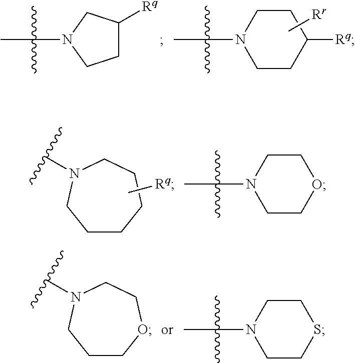
where R^{q} is —H and R^{r} is —H;
or a pharmaceutically acceptable salt, pharmaceutically acceptable prodrug, or pharmaceutically active metabolite thereof; and
(b) a pharmaceutically acceptable excipient.

18. A pharmaceutical composition according to claim 17, further comprising: an active ingredient selected from the group consisting of H₁ receptor antagonists, H₂ receptor antagonists, H₃ receptor antagonists, serotonin-norepinephrine reuptake inhibitors, selective serotonin reuptake inhibitors, noradrenergic reuptake inhibitors, non-selective serotonin re-uptake inhibitors, acetylcholinesterase inhibitors, and modafinil.

19. A method of treating a subject suffering from or diagnosed with a disease, disorder, or medical condition mediated by histamine H₃ receptor activity, comprising administering to the subject in need of such treatment an effective amount of a compound of Formula (I): 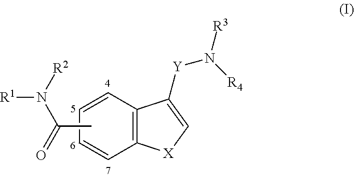
wherein
X is NR^{a} and Y is —CH₂— or X is S and Y is —CH₂— or —C(O)—;
where R^{a} is —H, methyl, —SO₂methyl;
the substituent —C(O)NR¹R² is bound at the 4-, 5-, 6-, or 7-position on Formula (I);
R¹ is —H and R² is —(CH₂)-pyridyl, where said pyridyl is unsubstituted or substituted with methyl;
or R¹ and R² taken together with the nitrogen to which they are attached form one of the following moieties: 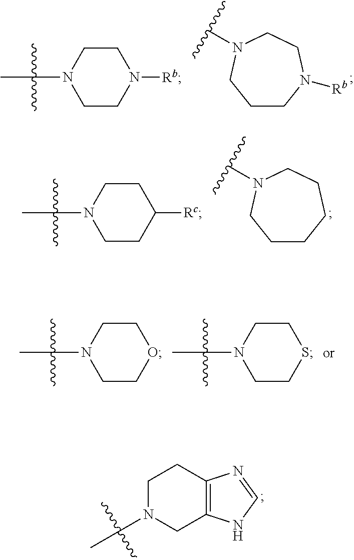
where R^{b} is isopropyl, cyclopropyl, or cyclobutyl; and
R^{c} is —H, hydroxymethyl, phenyl, or 1-pyrrolidin-2-onyl;
R³ and R⁴ taken together with the nitrogen to which they are attached form one of the following moieties: 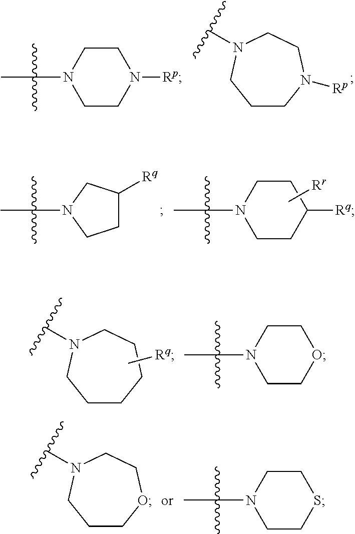
where R^{p} is isopropyl, acetyl, methylsulfonyl, C₃₋₅cycloalkyl, phenyl, —C(O)-phenyl, biphenyl, benzyl, benzhydryl, phenethyl, pyridyl, —C(O)-pyridyl, thiazolyl, or —C(O)-morpholinyl;
R^{q} is —H, —OH, phenyl, benzyl, —NR^{s}R^{t}, or —N(R^{s})C(O)R^{t};
where R^{s} and R^{t} are each independently —H or methyl;
or alternatively, R^{s} and R^{t} taken together with the nitrogen to which they are attached form piperidine; and
R^{r} is —H or —OH;
with the following provisos:
1) when
a) the substituent —C(O)NR¹R² is bound at the 5-position in Formula (I); and
b) R¹ and R² taken together with the nitrogen to which they are attached form one of the following moieties: 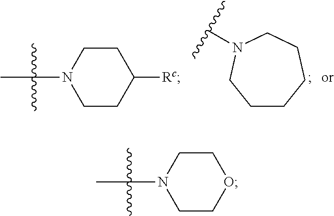
and
c) R^{c} is —H;
then R³ and R⁴ taken together with the nitrogen to which they are attached do not form one of the following moieties: 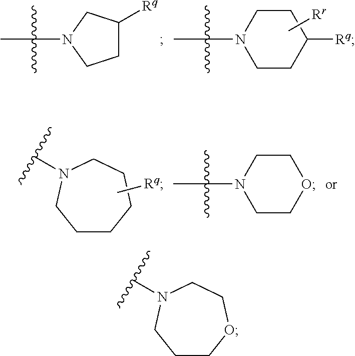
where R^{q} is —H and R^{r} is —H;
2) when
a) X is NR^{a}; and
b) the substituent —C(O)NR¹R² is bound at the 4- or 7-position on Formula (I);
then the substituents —C(O)NR¹R² and —YNR³R⁴ together comprise two nitrogens each of which is not adjacent to a carbonyl or sulfonyl group;
3) when
a) NR¹R² is 4-benzylpiperidin-1-yl; and
b) the substituent —C(O)NR¹R² is bound at the 5- or 6-position on Formula (I);
then R³ and R⁴ taken together with the nitrogen to which they are attached do not form one of the following moieties: 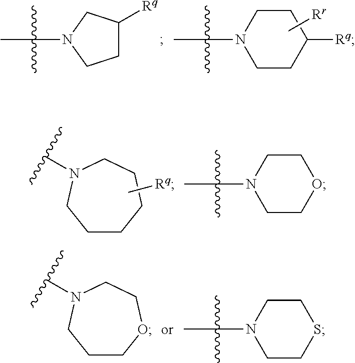
where R^{q} is —H and R^{r} is —H;
or a pharmaceutically acceptable salt, pharmaceutically acceptable prodrug, or pharmaceutically active metabolite thereof.

20. The method according to claim 19, wherein the disease, disorder, or medical condition is selected from the group consisting of: cognitive disorders, sleep disorders, psychiatric disorders, and other disorders.

21. The method according to claim 19, wherein the disease, disorder, or medical condition is selected from the group consisting of: dementia, Alzheimer's disease, cognitive dysfunction, mild cognitive impairment, pre-dementia, attention deficit hyperactivity disorders, attention-deficit disorders, and learning and memory disorders.

22. The method according to claim 19, wherein the disease, disorder, or medical condition is selected from the group consisting of: learning impairment, memory impairment, age-related cognitive decline, and memory loss.

23. The method according to claim 19, wherein the disease, disorder, or medical condition is selected from the group consisting of: insomnia, disturbed sleep, narcolepsy with or without associated cataplexy, cataplexy, disorders of sleep/wake homeostasis, idiopathic somnolence, excessive daytime sleepiness, circadian rhythm disorders, fatigue, lethargy, jet lag and REM-behavioral disorder.

24. The method according to claim 19, wherein the disease, disorder, or medical condition is selected from the group consisting of: sleep apnea, perimenopausal hormonal shifts, Parkinson's disease, multiple sclerosis, depression, chemotherapy, and shift work schedules.

25. The method according to claim 19, wherein the disease, disorder, or medical condition is selected from the group consisting of: schizophrenia, bipolar disorders, manic disorders, depression, obsessive-compulsive disorder, and post-traumatic stress disorder.

26. The method according to claim 19, wherein the disease, disorder, or medical condition is selected from the group consisting of: motion sickness, vertigo, benign postural vertigo, tinitus, epilepsy, migraine, neurogenic inflammation, neuropathic pain, Down Syndrome, seizures, eating disorders, obesity, substance abuse disorders, movement disorders, restless legs syndrome, eye-related disorders, macular degeneration, and retinitis pigmentosis.

27. The method according to claim 19, wherein the disease, disorder, or medical condition is selected from the group consisting of: depression, disturbed sleep, fatigue, lethargy, cognitive impairment, memory impairment, memory loss, learning impairment, attention-deficit disorders, and eating disorders.
